(19) United States
(12) Patent Application Publication
(10) Pub. No.: US 2024/0075093 A1
(43) Pub. Date: Mar. 7, 2024

(54) COMPOSITIONS AND METHODS OF TREATING A PI3K MEDIATED DISEASE

(71) Applicant: VIRGINIA TECH INTELLECTUAL PROPERTIES, INC., Blacksburg, VA (US)

(72) Inventors: Robert G. Gourdie, Roanoke, VA (US); Zhi Sheng, Roanoke, VA (US); Kevin J. Pridham, Roanoke, VA (US); Linda Jane Jourdan, Roanoke, VA (US)

(21) Appl. No.: 18/030,250

(22) PCT Filed: Oct. 9, 2021
(86) PCT No.: PCT/US2021/054331

§ 371 Date: Apr. 4, 2023

Related U.S. Application Data
(60) Provisional application No. 63/090,121, filed on Oct. 9, 2020.
(60) Provisional application No. 63/090,140, filed on Oct. 9, 2020.

(51) Int. Cl. A61K 38/00; A61K 31/495; A61K 45/06; A61P 35/00

(52) U.S. Cl. CPC A61K 38/005; A61K 31/495; A61K 45/06; A61P 35/00

(57) ABSTRACT

Described herein are compositions and formulations to treat and/or prevent a PI3K mediated disease or a symptom thereof in a subject in need thereof. In some embodiments, such compositions include engineered peptides capable of targeting PI3K or Connexin 43. Also described herein are methods of treating and/or preventing PI3K mediated disease or a symptom thereof. In some embodiments, the PI3K mediated disease is a cancer.

### CROSS-REFERENCE TO RELATED APPLICATIONS

This application is application is the U.S. National Stage Application under 35 U.S.C. § 371 of Patent Cooperation Treaty Application No.: PCT/US2021/054331, filed on Nov. 2, 2021, which claims the benefit of and priority to U.S. Provisional Patent Application No. 63/090,121, filed on Oct. 9, 2020 entitled “ACT1 and ACT11 Peptide Variant Combination Therapies for Treatment of Disease” and to U.S. Provisional Patent Application No. 63/090,140, filed on Oct. 9, 2020 entitled “P110Beta Targeting Peptides, Formulations, and Uses Thereof” the contents of which are incorporated by reference herein in their entireties.

### STATEMENT REGARDING FEDERALLY SPONSORED RESEARCH

This invention was made with government support under Grant No. CA216768, CA245631, and HL056728 awarded by National Institutes of Health. The government has certain rights in the invention.

### SEQUENCE LISTING

This application contains a sequence listing filed in electronic form as an ASCII.txt file entitled VTIP-0295US_ST25.txt, created on Sep. 7, 2023, and having a size of 54,427 bytes. The content of the sequence listing is incorporated herein in its entirety.

### TECHNICAL FIELD

The subject matter disclosed herein is generally directed to treating PI3K mediated diseases, such as cancer.

### BACKGROUND

A major problem in treating cancer is resistance to chemotherapy. For example, glioblastoma (GBM) and some melanoma patients are often resistant to the frontline chemotherapeutics, such as Temozolomide (TMZ) in the case of GBM. Although the mechanisms underlying chemoresistance in cancer are not fully appreciated, it is believed that aberrant PI3K expression, function, and/or activity is involved. As such, there exists a need for improved therapeutics and strategies for treating cancers, such as those whose etiology, recurrence, and/or chemoresistance involves PI3K.

Citation or identification of any document in this application is not an admission that such a document is available as prior art to the present invention.

### SUMMARY

Described in several example embodiments herein are engineered peptides comprising a p110beta targeting peptide and a delivery moiety, wherein the delivery moiety is coupled to the p110beta targeting peptide.

In certain example embodiments, the p110beta targeting peptide is capable of selectively binding p110beta or a complex thereof, selectively inhibiting p110beta activity, or both.

In certain example embodiments, the p110beta targeting peptide comprises an amino acid sequence that is about 95 to 100 identical to SEQ ID NO: 3 or is a homologue thereof, or functional variant thereof.

In certain example embodiments, the delivery moiety is a cell penetrating peptide.

In certain example embodiments, the cell penetrating peptide has a sequence identical to SEQ ID NO: 4, SEQ ID NO: 6, or any one of SEQ ID NOs: 110-222.

In certain example embodiments, one or more amino acids of the engineered peptide comprises one or more ester linked groups.

Described in certain example embodiments are pharmaceutical formulation comprising an engineered peptide of any one of the previous paragraphs and elsewhere herein and a pharmaceutically acceptable carrier.

In certain example embodiments, the pharmaceutical formulation further comprises a connexin 43 inhibitor; a chemotherapeutic; an immune checkpoint inhibitor; or any combination thereof.

In certain example embodiments, the Cx43 inhibitor is a biologic molecule-based inhibitor, a chemical molecule inhibitor, a small molecule inhibitor, an RNAi-based inhibitor, or a genetic modifier-based inhibitor.

In certain example embodiments, the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In certain example embodiments, the chemotherapeutic is not effective to treat a cancer or a chemotherapeutic resistant cancer having overexpression of p110beta when used alone.

In certain example embodiments, the chemotherapeutic is temozolomide.

Described in certain example embodiments herein are delivery vesicles, such as exosmoes, comprising, an engineered peptide of any one of the prior paragraphs and/or elsewhere herein.

In certain example embodiments, the delivery vesicle, such as an exosome, further comprises a connexin 43 inhibitor; a chemotherapeutic; an immune checkpoint inhibitor; or any combination thereof.

In certain example embodiments, the Cx43 inhibitor is a biologic molecule-based inhibitor, a chemical molecule inhibitor, a small molecule inhibitor, an RNAi-based inhibitor, or a genetic modifier-based inhibitor. In certain example embodiments, the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In certain example embodiments, the chemotherapeutic is not effective to treat a cancer or a chemotherapeutic resistant cancer having overexpression of p110beta when used alone. In certain example embodiments, the chemotherapeutic is temozolomide.

In certain example embodiments, the delivery vesicle is a milk exosome.

Described in certain example embodiments herein are methods of treating a PI3K mediated disease or a symptom thereof in a subject in need thereof, the method comprising administering, to the subject in need thereof, an engineered peptide as described in any one of the preceding paragraphs and/or elsewhere herein.

In certain example embodiments, the PI3K mediated disease is a cancer, optionally a chemotherapy resistant cancer. In certain example embodiments, the cancer is characterized at least in part by overexpression of p110beta. In certain example embodiments, the cancer is glioblastoma or melanoma.

Described in certain example embodiments herein are methods of treating a PI3K mediated disease or a symptom thereof in a subject in need thereof, the method comprising administering, to the subject in need thereof, a pharmaceutical formulation comprising an engineered peptide as in any one of the preceding paragraphs and/or elsewhere herein and a pharmaceutically acceptable carrier.

In certain example embodiments, the pharmaceutical formulation further comprises a connexin 43 inhibitor; a chemotherapeutic; an immune checkpoint inhibitor; or any combination thereof.

In certain example embodiments, the Cx43 inhibitor is a biologic molecule-based inhibitor, a chemical molecule inhibitor, a small molecule inhibitor, an RNAi-based inhibitor, or a genetic modifier-based inhibitor. In certain example embodiments, the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In certain example embodiments, the chemotherapeutic is a chemotherapeutic used to treat a cancer having overexpression of p110beta. In certain example embodiments, the chemotherapeutic is temozolomide. In certain example embodiments, the PI3K mediated disease is a cancer, optionally a chemotherapy resistant cancer. In certain example embodiments, the cancer is characterized at least in part by overexpression of p110beta. In certain example embodiments, the cancer is glioblastoma or melanoma.

Described in certain example embodiments herein are methods of treating a PI3K mediated disease or a symptom thereof in a subject in need thereof, the method comprising administering, to the subject in need thereof, a delivery vesicle, such as an exosome, of any one of the preceding paragraphs or a pharmaceutical formulation thereof.

In certain example embodiments, the exosome further comprises a connexin 43 inhibitor; a chemotherapeutic; an immune checkpoint inhibitor; or any combination thereof.

In certain example embodiments, the Cx43 inhibitor is a biologic molecule-based inhibitor, a chemical molecule inhibitor, a small molecule inhibitor, an RNAi-based inhibitor, or a genetic modifier-based inhibitor. In certain example embodiments, the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In certain example embodiments, the chemotherapeutic is not effective to treat a cancer or a chemotherapeutic resistant cancer having overexpression of p110beta when used alone. In certain example embodiments, the chemotherapeutic is temozolomide.

In certain example embodiments, the PI3K mediated disease is a cancer, optionally a chemotherapy resistant cancer. In certain example embodiments, the cancer is characterized at least in part by overexpression of p110beta. In certain example embodiments, the cancer is glioblastoma or melanoma.

Described in several example embodiments herein are kits comprising an engineered peptide of any one of the preceding paragraphs and/or elsewhere herein, a pharmaceutical formulation as in any one of the preceding paragraphs and/or elsewhere herein, a delivery vesicle, such as an exosome, as in any one of the preceding paragraphs, or any combination thereof.

Described in several example embodiments herein are pharmaceutical formulations comprising a connexin 43 inhibitor; a chemotherapeutic; and optionally a PI3K inhibitor; and a pharmaceutically acceptable carrier.

In certain example embodiments, the Cx43 inhibitor is a biologic molecule-based inhibitor, a chemical molecule inhibitor, a small molecule inhibitor, an RNAi-based inhibitor, or a genetic modifier-based inhibitor. In certain example embodiments, the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In certain example embodiments, the PI3K inhibitor is a selective p110beta inhibitor. In certain example embodiments, the selective p110beta inhibitor is a biologic molecule-based inhibitor, a chemical molecule inhibitor, a small molecule inhibitor, an RNAi-based inhibitor, or a genetic modifier-based inhibitor. In certain example embodiments, the selective p110beta inhibitor is an engineered peptide comprising a p110beta targeting peptide; and a delivery moiety, wherein the delivery moiety is coupled to the p110beta targeting peptide, wherein the engineered peptide optionally comprises one or more ester-linked groups.

In certain example embodiments, the p110beta targeting peptide is capable of selectively binding p110beta or a complex thereof, selectively inhibiting p110beta activity, or both. In certain example embodiments, the p110beta targeting peptide comprises an amino acid sequence that is about 95 to 100 identical to SEQ ID NO: 3 or is a homologue thereof, or functional variant thereof.

In certain example embodiments, the delivery moiety is a cell penetrating peptide. In certain example embodiments, the cell penetrating peptide has a sequence identical to SEQ ID NO: 4, SEQ ID NO: 6, or any one of SEQ ID NOs. 110-222.

In certain example embodiments, the PI3K inhibitor is a biologic molecule-based inhibitor, a chemical molecule inhibitor, a small molecule inhibitor, an RNAi-based inhibitor, or a genetic modifier-based inhibitor.

In certain example embodiments, the chemotherapeutic is not effective to treat a cancer or a chemotherapeutic resistant cancer having overexpression of p110beta, Cx43, or both when used alone. In certain example embodiments, the chemotherapeutic is temozolomide.

Described in certain example embodiments herein are delivery vesicles, such as exosomes, comprising a connexin 43 inhibitor; a chemotherapeutic; and optionally a PI3K inhibitor.

In certain example embodiments, the Cx43 inhibitor is a biologic molecule-based inhibitor, a chemical molecule inhibitor, a small molecule inhibitor, an RNAi-based inhibitor, or a genetic modifier-based inhibitor. In certain example embodiments, the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In certain example embodiments, the PI3K inhibitor is a selective p110beta inhibitor. In certain example embodiments, the selective p110beta inhibitor is a biologic molecule-based inhibitor, a chemical molecule inhibitor, a small molecule inhibitor, an RNAi-based inhibitor, or a genetic modifier-based inhibitor. In certain example embodiments, the selective p110beta inhibitor is an engineered peptide comprising a p110beta targeting peptide; and a delivery moiety, wherein the delivery moiety is coupled to the p110beta targeting peptide, wherein the engineered peptide optionally comprises one or more ester-linked groups.

In certain example embodiments, the p110beta targeting peptide is capable of selectively binding p110beta or a complex thereof, selectively inhibiting p110beta activity, or both. In certain example embodiments, the p110beta targeting peptide comprises an amino acid sequence that is about 95 to 100 identical to SEQ ID NO: 3 or is a homologue thereof, or functional variant thereof. In certain example embodiments, the delivery moiety is a cell penetrating peptide. In certain example embodiments, the cell penetrating peptide has a sequence identical to SEQ ID NO: 4, SEQ ID NO: 6, or any one of SEQ ID NOs: 110-222.

In certain example embodiments, the PI3K inhibitor is a biologic molecule-based inhibitor, a chemical molecule inhibitor, a small molecule inhibitor, an RNAi-based inhibitor, or a genetic modifier-based inhibitor. In certain example embodiments, the chemotherapeutic is not effective to treat a cancer or a chemotherapeutic resistant cancer having overexpression of p110beta, Cx43, or both when used alone. In certain example embodiments, the chemotherapeutic is temozolomide.

Described in certain embodiments herein are methods of treating a PI3K mediated disease or a symptom thereof in a subject in need thereof, the method comprising administering, to the subject in need thereof, a pharmaceutical formulation as in any one of the preceding paragraphs and/or elsewhere herein or a delivery vesicle, such as an exosome, as in any one the preceding paragraphs and/or elsewhere herein, or an amount of a connexin 43 inhibitor, an amount of a chemotherapeutic, and an amount of a PI3K inhibitor.

In certain example embodiments, the Cx43 inhibitor is a biologic molecule-based inhibitor, a chemical molecule inhibitor, a small molecule inhibitor, an RNAi-based inhibitor, or a genetic modifier-based inhibitor. In certain example embodiments, the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In certain example embodiments, the PI3K inhibitor is a selective p110beta inhibitor. In certain example embodiments, the selective p110beta inhibitor is a biologic molecule-based inhibitor, a chemical molecule inhibitor, a small molecule inhibitor, an RNAi-based inhibitor, or a genetic modifier-based inhibitor. In certain example embodiments, the selective p110beta inhibitor is an engineered peptide comprising a p110beta targeting peptide; and a delivery moiety, wherein the delivery moiety peptide is coupled to the p110beta targeting peptide, wherein the engineered peptide optionally comprises one or more ester-linked groups. In certain example embodiments, the p110beta targeting peptide comprises an amino acid sequence that is about 95 to 100 identical to SEQ ID NO: 3 or is a homologue thereof, or functional variant thereof.

In certain example embodiments, the delivery moiety is a cell penetrating peptide. In certain example embodiments, the cell penetrating peptide has a sequence identical to SEQ ID NO: 4, SEQ ID NO: 6, or any one of SEQ ID NOs: 110-222.

In certain example embodiments, the PI3K inhibitor is a biologic molecule-based inhibitor, a chemical molecule inhibitor, a small molecule inhibitor, an RNAi-based inhibitor, or a genetic modifier-based inhibitor.

In certain example embodiments, the chemotherapeutic is not effective to treat a cancer or a chemotherapeutic resistant cancer having overexpression of p110beta, Cx43, or both when used alone. In certain example embodiments, the chemotherapeutic is temozolomide.

In certain example embodiments, the PI3K mediated disease is a cancer, optionally a chemotherapy resistant cancer. In certain example embodiments, the cancer is characterized at least in part by overexpression of p110beta, Cx43, or both. In certain example embodiments, the cancer is glioblastoma or melanoma.

Described in certain example embodiments are kits comprising the pharmaceutical formulation of any one of the preceding paragraphs and/or elsewhere herein, an exosome of any one of the preceding paragraphs and/or elsewhere herein, or an amount of or an amount of a connexin 43 inhibitor, an amount of a chemotherapeutic, and an amount of a PI3K inhibitor.

Described in certain example embodiments herein are polynucleotides encoding an engineered peptide as described in any one of the preceding paragraphs.

Described in certain example embodiments herein are vectors, optionally an expression vector, comprising a polynucleotide of the preceding paragraph and/or elsewhere herein.

Described in certain example embodiments herein is a cell or cell population comprising a polynucleotide of any one of the preceding paragraphs and/or elsewhere herein, a vector of any one of the preceding paragraphs and/or elsewhere herein or both.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows a graph demonstrating expression of PIK3C isoforms, particularly PIK3CB, in different cancer types.

FIG. 2A-2E shows graphs demonstrating mRNA expression of PIK3CB in melanoma cells and skin fibroblasts. Graphs were assembled from data from DepMap database.

FIG. 3 demonstrates the effect of Ante-Selectide-18 on PI3K/AKT Signaling. SF295 cells were treated with 200 μM Ante-Selectide-18 for 4 days with a replenishment of fresh peptide every day. The vehicle was used as the control. PI3K/AKT signaling was analyzed using immunoblotting. Three signaling pathways (GSK3b, RAC1/CDC42, and MTOR) are shown. ACTB is the loading control.

FIG. 4 demonstrates cellular Uptake of FITC-Selectide-18. SF295 and LN229 cells were treated with treated with 100 μM FITC-Selectide-18 for 1 hour and nuclei were stained by Hoechst33342. FITC (green fluorescence, as represented in greyscale) and Hoechst33343 (blue fluorescence, as represented in greyscale) were imaged using an inverted fluorescence microscope. The inset figures in Overlay show a single cell.

FIG. 5 shows a graph demonstrating activity of p110b. The activity of the p110b PI3K catalytic subunit was monitored via ELISA upon increasing doses of Ante-Selectide-18. The IC50 was calculated to be 105 nM based off this activity assay.

FIG. 6A-6C shows (FIG. 6A) ante-Selectide-18 Only Inhibits p110b, not Other PI3K Subunits. The activity of PI3K subunits was monitored via ELISA with isolated PI3K protein subunits treated with no peptide, 100 nM Ante-Selectide-18, 100 nM Ante-Scramble-18 control peptide, or 100 nM TGX-221. Relative activity is the ratio of respective treated groups to their non-treated controls. FIG. 6B shows a diagram demonstrating the selectivity of the ante-selectide-18. FIG. 6C shows ribbon models of p110 isoforms with and without selectide-18 binding modeled.

FIG. 7A-7C demonstrates that p110β controls PI3K/AKT activity in GBM. Cas9 and guide RNAs (gRNA) of non-targeting (NT), PIK3CB (FIG. 7A), PIK3CA (FIG. 7B), or PIK3CD (FIG. 7C) were virally delivered into GBM cells. AKT activity is indicated by levels of pAKTS473 and pAKTT308. Trace amount of p110 proteins seen in the knockout cells was from uninfected cells.

FIG. 8A-8D demonstrates p110β regulates GBM cell migration. (FIG. 8A) Wound-healing assay. Cells were plated confluently and then treated with PI3K isoform-selective inhibitors at 8 μM for 24 hours. Cells were imaged at 0- and 24-hour time point. Images of 24-hour time point are shown. Scale bar: 50 m. (FIG. 8B-8D) Quantification of migration distance. Images from three different areas were analyzed using Image J. Migration distances were obtained by subtracting 24-hour measurements with those of 0-hour.

FIG. 9A-9C demonstrates invasion/survival signaling downstream of p110β. (FIG. 9A) SF295 cells were treated with 8 μM TGX-221. Invasion molecules RAC1/CDC42 and MMPs are shown. Images were quantified using Image J. 0 h time point is set as 1.0. (FIG. 9B) SF295 cells were treated with 25 μM TGX-221 for 96 h. Apoptosis molecules FOXO3, BIM, and c-CASP3 are shown. (FIG. 9C) Invasion/survival signaling downstream of p110p.

FIG. 10A-10H demonstrates structures amongst p110 proteins. (FIG. 10A) Co-immunoprecipitation (co-IP). p110β and p110α were pulled down in U87MG. p85s and p110s were detected using immunoblotting. (FIG. 10B) p110β 3D simulation using the SWISS-MODEL program. C2 domain, RAS-binding domain (RBD), p85-binding domain (PBD), and helical & kinase domain (HKD) are shown. Box: the groove between C2 and HKD. (FIG. 10C) Alignment of p110s. Protein sequences were aligned using the COBLAT program. p110 C2 domains are shown in the top panel. Red bars: homologous residues; Grey bars: non-homologous residues; Darker lines: missing residues. TKKSTKTINPSKYQTIRK (SEQ ID NO: 3) found exclusively in p110β C2 are shown in bottom panel. (FIG. 10D) Interactions between p85β iSH2 domain (blue as represented in greyscale) and p110β C2 domain (green as represented in greyscale). (FIG. 10E-10H) 3D shapes (boxes) between C2 and HKD in p110 proteins.

FIG. 11A-11H demonstrates the 18-amino-acid motif shapes p110β C2 domain. p110 proteins were remodeled using the SWISS-MODEL program. Depletion of TKKSTKTINPSKYQTIRK (SEQ ID NO: 3) (C2-18aa) in p110 converts a loose conformation (FIG. 11A) into a dense conformation (FIG. 11B). Addition of this motif in p110α (C2+18aa) and p110δ (C2+18aa) turns dense conformations (FIG. 11C, 11E) into loose or looser conformations (FIG. 11D, 11F, 11H).

FIG. 12A-12D demonstrates p110βC2in inactivates AKT and inhibits GBM cell survival. (FIG. 12A) p110βC2in. CPP: cell-penetrating peptide. Simulated p110βC2in (rainbow as represented in greyscale) is shown. (FIG. 12B) FITC-p110βC2in in LN229/GSCs was imaged using a fluorescence microscope. Upper: FITC; lower: bright view. (FIG. 12C) PI3K/AKT activity affected by 100 μM p110βC2in (+) or C2Scramble-1 (−). (FIG. 12D) MTS Cell viability assay. Cells were treated with 50 μM peptides for 4 days. Student t test was used to determine P values.

FIG. 13A-13B demonstrates (FIG. 13A) U87MG cells were transduced with either a non-silencing (NS) shRNA or an shRNA of PI3K isoforms in combination with 50 uM TMZ. (FIG. 13B) GBM cells were treated with 20 uM TGX-221 and/or 50 uM TMZ for 4 days. Cell viability was measured by the MTS assay. P values were determined using the student t test or one-way ANOVA. Error bars represent three independent experiments.

FIG. 14 shows a table demonstrating p110βhigh and p110βlow GBM cells.

FIG. 15 shows a table demonstrating p110β protein levels inversely correlate with patient survival.

FIG. 16 shows a table of PI3K inhibitors.

FIG. 17A-17B p110β-mCherry. (FIG. 17A) pCMV-p110β-mCherry was transfected into U87MG cells. Cells were imaged using a confocal microscope. (FIG. 17B) Immunoblotting of p110β, pAKTS473, and 3-actin (ACTB).

FIG. 18A-18C demonstrates 3D invasion of GSCs in Matrigel. (FIG. 18A) Transwell cell invasion assay. LN229/GSCs were plated onto a Matrigel with reduced growth factor in a transwell plate. Cells invaded into the outer membrane of inserts were stained by crystal violet. VTC-036/GSCs (FIG. 18B) or LN229/GSCs/GFP+ (FIG. 18C) were cultured in Matrigel for 2 to 3 weeks and imaged using a light microscope or an inverted fluorescence microscope. Invasive cells were highlighted in yellow or magenta as represented in greyscale, respectively.

FIG. 19A-19B demonstrates Confocal IF and STORM. p110 was stained by a p110 antibody followed by a Texas red-conjugated secondary antibody and U87MG cells were imaged using a confocal microscope (FIG. 19A). The gap junction protein connexin 43 (Cx43) was immune-stained and visualized using a STORM microscope (FIG. 19B).

FIG. 20A-20B demonstrates infiltrative GBM cells in the mouse brain. (A) 105 GS9-6/GSCs (derived from GBM tumor specimen) were injected into the brain of a NOD scid gamma mouse. The brain tumor (arrow) was imaged using MRI and shown by H&E (grey arrows). (B) 106 U251/GFP+ cells were injected into the brain of a NOD scid gamma mouse. 4 weeks later, the mouse brain was collected. Brain sections were stained with DAPI. Images were taken using a confocal microscope. Grey arrows: infiltrative human U251/GFP+ cells. Grey boxes: mouse normal brain cells.

FIG. 21 shows microscopic images GL261/GSC/GFP+ cells. GL261/GSC cells were transfected with a GFP plasmid and sorted by FACS. GSCs were maintained in stem cell culture media and imaged using a fluorescence microscope. Left: bright view; Right: GFP.

FIG. 22 shows a heatmap demonstrating PIK3CB expression in various melanoma cell lines.

FIG. 23 shows a table with PIK3 isoform expression in various melanoma cell lines.

FIG. 24 shows a graph demonstrating viability of cell lines treated with selectide-18 or untreated.

FIG. 25 demonstrates structural differences among different p110s.

FIG. 26 demonstrates structural differences in p110 C2 domains.

FIG. 27A-27G demonstrates that selectide-18 is a p110beta-selective inhibitor and is effective in e.g., p110beta^{high} glioblastoma.

FIG. 28 further demonstrates that that selectide-18 is a p110beta-selective inhibitor and is effective in e.g., p110beta^{high} glioblastoma.

FIG. 29A-29G demonstrates that Cx43 is expressed at the highest level among all connexins in GBM. mRNA levels of connexins in GBMs from The Cancer Genome Atlas (TCGA; FIG. 29A), Murat (FIG. 29B), Rembrandt (FIG. 29C), Chinese Glioma Gene Atlas (CGGA; FIG. 29D), and Gravendeel (FIG. 29E). Shown are average reads of microarray or RNAseq. Cx43 is presented as dark grey bars with dark grey data points. Other connexins are labeled as light grey bars and light grey data points. Error bars are either standard deviations or standard errors. (FIG. 29F) Staining scores of connexins in high-grade glioma. Case numbers with high or not high levels of connexins are shown. (FIG. 29G) Histological images of connexins in a high-grade glioma tumor. Inset images (highlighted in grey) were cropped from original images in order to highlight immunostaining details. GBM datasets were retrieved from cBioPortal, GlioVis, or CGGA data portal. Immunostaining results of high-grade glioma were obtained from the Human Protein Atlas. Statistical analyses: One-Way ANOVA with Dunnett test for correction of multiple comparisons and Fisher's exact test. ns: not significant; ****: P<0.0001.

FIG. 30A-30D demonstrates that Cx43, but not other connexins, correlates with GBM poor prognosis and chemoresistance. GBM datasets were retrieved from cBioPortal, GlioVis, or CGGA data portal. Immunostaining results of high-grade glioma were obtained from the Human Protein Atlas. (FIG. 30A) Kaplan-Meier analysis in the TCGA HG-U133A microarray dataset. Patients were divided into Cx43-high (light grey; top 25 percentile) or Cx43-low (darker grey; bottom 25 or 75 percentile) based upon Cx43 mRNA levels in primary, secondary, and recurrent GBM (All GBM), primary GBM only (Primary GBM), MGMT promoter methylated primary GBM (MGMT−), MGMT promoter unmethylated primary GBM (MGMT+), or recurrent GBM only (Recurrent GBM). Case number (n), mean survival time in months (m), and log-rank P values are shown. Light grey or darker grey shadows represent 95% confidence interval of Cx43-high or Cx43-low group, respectively. (FIG. 30B) Cox univariate analysis in the TCGA HG-U133A microarray dataset. The Cox univariate analysis employs the Cox proportional hazards model to yield a hazard ratio that indicates risk levels of death in patients with high levels of connexins compared to those with low levels. The resulting P value determines significance of hazard ratio. Cx43 is highlighted in dark grey. (FIG. 30C) Kaplan Meier analysis in TCGA HG-U133A and Murat GBM. MGMT-deficient primary GBMs were divided into Cx43-high or Cx43-low group as described above. Patients treated with radiation alone (Radio; light grey) were compared to patients treated with both radiation and TMZ (Radio+TMZ; darker grey). (FIG. 30D) Cox univariate analysis in TCGA HG-U133A, Murat GBM, and CGGA recurrent GBM. MGMT-deficient primary GBMs or recurrent GBMs were divided into Cx43-high or Cx43-low group. One-Way ANOVA was used to determine statistical significance. *: P<0.05. ****: P<0.0001. ns: not significant.

FIG. 31A-31J demonstrates that Cx43 blockade inactivates PI3K. (FIG. 31A) Signaling pathways affected by aCT1. Cx43-high U87MG cells were treated with 100 μM αCT1 or 50 μM TMZ for 4 days. pAKT-5473, pcRAF-5338, pERK-T202/T204), and pSRC-T416 were analyzed using immunoblotting. Glyceraldehyde 3-phosphate dehydrogenase (GAPDH) was the loading control. Band intensities were quantified using Image J. Vehicle was set as 1.0 and each treatment was normalized to the vehicle. (FIG. 31B) PI3K signaling upon depletion of Cx43. U87MG and Cx43-low A172 cells were treated with a non-silencing short hairpin RNA (NS shRNA) or a Cx43 shRNA. U87MG cells treated with NS shRNA were set as 1.0. f-actin (ACTB) was the loading control. Pearson coefficient correlation analysis between protein levels of Cx43 and pAKT-S473 in 6 MGMT− GBM cell lines (FIG. 31C), mRNA levels of Cx43 and protein levels of pAKT-S473 or pAKT-T308 in MGMT− patients (FIG. 31D), or mRNA levels of connexins and protein levels of pAKT-S473 (FIG. 31E) and pAKT-T308 (FIG. 31F) in MGMT− GBMs. The Pearson correlation coefficient (r) and P value that determines statistical significance of the coefficient are shown. Cell line data were retrieved from previous studies (Murphy et al. Cancer Res 76:139-149 (2016) and Wu et al. Neuro Oncol. Doi:10.1093/neuonc/noab003 (2021)). (FIG. 31G) Expression of PIK3CA-E545K (an active PI3K mutant). U87MG cells were transfected with pBABE or pBABE-PIK3CA-E545K encoding PIK3CA-E545K followed by the treatment of 100 μM TMZ. Dimethyl Sulfoxide (DMSO) is the vehicle control. (FIG. 31H) The effect of PIK3CA-E545K on the αCT1/TMZ-induced growth inhibition. Transfected cells were treated with a combination of 100 μM αCT1 and/or 100 μM TMZ for 6 days. Cell viability was measured using the MTS viability assay. Percentages of viability were obtained by normalizing the MTS readings of treatment groups to that of DMSO. (FIG. 31I) The effect of ERK2-L73PS151D on the αCT1/TMZ-induced growth inhibition. U87MG cells were transfected with pCMV5 or pCMV5-ERK2-L73PS151D (encoding an active ERK2 mutant) followed by the treatment of αCT1 or antennapedia peptide (ANT; the control peptide for αCT1) and/or TMZ. (FIG. 31J) The effect of SRC-Y527F on the αCT1/TMZ-induced growth inhibition. U87MG cells were transfected with pBABE or pBABE-SRC-Y527F (encoding an active SRC mutant) followed by the treatment of αCT1 or ANT and/or TMZ. RNA sequencing (RNAseq) data and results of reverse phase protein array (RPPA) were retrieved from the TCGA database. Student's t test was used to determine statistical significance. *: P<0.05; ****: P<0.0001.

FIG. 32A-32F demonstrates that Cx43 activates PI3K through selectively binding to the PI3K catalytic subunit β. (FIG. 32A) The effect of Gap27 on PI3K signaling. U87MG cells were treated with 100 μM TMZ or 100 μM Gap27. (FIG. 32B) ATP release from Cx43-high/MGMT−/TMZ-resistant SF295 cells. Cells were treated with 100 μM αCT1. Culture media were collected at different time points. ATP was measured using a colorimetric assay. One-way ANOVA was used to determine statistical significance. (FIG. 32C) Glutamate release in Cx43-low/MGMT−/TMZ-sensitive LN229 or Cx43-high/MGMT−/TMZ-resistant LN229/GSC cells. Cells were treated with 100 μM TMZ and/or 100 μM αCT1. Glutamate in culture media was determined using a colorimetric assay. (FIG. 32D) ATP release in LN229 and LN229/GSC cells. (FIG. 32E) ATP within SF295 cells. (FIG. 32F) Glutamate within LN229 and LN229/GSC cells. GSC: glioblastoma stem cells.

FIG. 33A-33I demonstrates that Cx43 activates PI3K through selectively binding to the PI3K catalytic subunit β. Pearson coefficient Correlation between protein levels of Cx43 and PI3K catalytic subunits in 6 MGMT− GBM cell lines (FIG. 33A), mRNA levels of Cx43 and PI3K catalytic subunits in MGMT− GBM patients (FIG. 33B), mRNA levels of PIK3CB and connexins in MGMT− GBM patients (FIG. 33C), protein levels of p110 proteins and IC50s of TMZ in 6 MGMT− GBM cell lines (FIG. 33D), or protein levels of pAKT-5473 and IC50s of TMZ in 6 MGMT− GBM cell lines (FIG. 33E). Cell line data were retrieved from previous studies (Murphy et al. Cancer Res 76:139-149 (2016) and Wu et al. Neuro Oncol. Doi:10.1093/neuonc/noab003 (2021)). RNAseq data were retrieved from the TCGA database. The Pearson correlation coefficient r and corresponding p are shown. Co-immunoprecipitation of Cx43 and p110β (FIG. 33F), p110α (FIG. 33G), or p110δ (FIG. 33H) in U87MG cells. (FIG. 33I) Co-immunoprecipitation of Cx43 and p110β in U87MG cell lysates treated with 100 μM αCT1. αCT1 is about 3 kDa and recognized by the Cx43 antibody. IP: immunoprecipitation. Rabbit IgG was used as the control.

FIG. 34A-34K demonstrates that a combination of αCT1 and TGX-221 overcomes TMZ resistance in vitro and in vivo. (FIG. 34A) The effect of the αCT1/TGX-221/TMZ combo in Cx43/p110f-high/MGMT−/TMZ-resistant SF295 and VTC-103 cells. Cells were treated with 50 μM TMZ, 20 μM TGX-221, and/or 30 μM αCT1 including single agents, double combinations and the αCT1/TGX-221/TMZ combo. This scheme has been repeated in experiments presented hereafter. Cell viability was determined using the MTS viability assay. Percentages of cell viability were obtained by normalizing the MTS readings of treatment groups to that of DMSO group. (FIG. 34B) Scores of Excess Over Bliss (EOB) calculated using the Bliss Independence model. The drug combination is synergistic if EOB is more than 0%, additive if EOB equals to 0%, or antagonistic if EOB is less than 0%. (FIG. 34C) The effect of the αCT1/TGX-221/TMZ in Cx43/p110β-low/MGMT−/TMZ-sensitive LN229 and TMZ-resistant VTC-001 cells. (FIG. 34D) EOB scores of drug combinations in LN229 and VTC-001 cells. (FIG. 34E) Caspase 3/7 activity in VTC-103 and VTC-001 cells. The activity of cleaved caspase 3/7 (active) was determined using a luminescence assay. Shown are luminescence readings. (FIG. 34F) EOB scores of drug combinations in VTC-103 and VTC-001 cells. (FIG. 34G) The effect of αCT1/TGX-221/TMZ combo on SF295 xenograft tumors. SF295 cells were subcutaneously injected into immuno-deficient mice. 8 days later, mice were treated with TMZ, TGX-221, or αCT1 through intraperitoneal or intratumoral injection every other day until day 18. Tumor volumes are shown. (FIG. 34H) EOB scores of drug combinations in SF295 tumors at different days. (FIG. 34I) The effect of shRNA of Cx43 or PI3K catalytic subunits on the TMZ sensitivity of SF295 cells. Cells were transfected with NS shRNA or shRNA of Cx43, PIK3CA, PIK3CB, or PIK3CD followed by the treatment of 50 μM TMZ. Cell viability was determined using the MTS viability assay. Percentages of cell viability were obtained by normalizing the MTS readings of treatment groups to that of shNS group. (FIG. 34J) EOB scores of drug combinations in SF295 cells. (FIG. 34K) A model illustrating the mechanism of Cx43-induced MGMT-independent TMZ resistance and the model of action of the triple combination. One-way ANOVA with Dunnett test for correcting multiple comparisons or Student's t test was used to determine statistical significance. *: P<0.05; ns: not significant. Drug combinations with strong synergistic effect were marked in grey.

FIG. 35 shows a table demonstrating the nomenclature of connexins. Information regarding gene symbols and aliases was retrieved from GeneCards (https://www.genecards.org).

FIG. 36 shows a table demonstrating levels of Cx43, pAKT-5473, p110β, MGMT and TMZ IC50. Data were retrieved from previous studies (Murphy et al. Cancer Res 76:139-149 (2016) and Wu et al. Neuro Oncol. Doi:10.1093/neuonc/noab003 (2021)).

FIG. 37A-37D demonstrates mRNA levels of connexins in GBM. Gene expression data were retrieved from cBioPortal, GlioVis, or the Cancer Dependency Map (DepMap). Shown are mRNA levels of connexins in the TCGA Agilent-4502A microarray (FIG. 37A), the TCGA RNAseq (FIG. 37B), the LeeY GBM dataset (FIG. 37C), and DepMap GBM cell lines (FIG. 37D). Case numbers (n) are also shown. Error bars represent standard deviations. Cx43 is highlighted in red and other connexins are in green. Individual data points are also shown (purple for Cx43 and yellow for other connexins). P values were obtained using One-Way ANOVA with Dunnett test for correction of multiple comparisons. ****: P<0.0001.

FIG. 38A-38B demonstrates levels of connexins in high-grade glioma. Immunohistochemical staining images of high-grade glioma were retrieved the Human Protein Atlas. Images of two patient specimens are shown in FIGS. 38A and 38B, respectively. Inset figures depict details of immunostaining. Levels of staining are highlighted in red (Cx43) or in green (other connexins).

FIG. 39A-39B demonstrates results from a Kaplan-Meier analysis and Cox univariate analysis in the TCGA Agilent-4502A dataset. Data were retrieved from cBioportal. Patients were divided into Cx43-high (red, top 25 percentile) and Cx43-low (blue, bottom 25 or 75 percentile) based upon Cx43 mRNA levels in primary, secondary, and recurrent GBM (All GBM), primary GBM only (Primary GBM), MGMT-deficient primary GBM (MGMT−), MGMT-expressing primary GBM (MGMT+), or recurrent GBM only (Recurrent GBM). Kaplan-Meier analysis (FIG. 39A) and Cox univariate analysis (FIG. 39B) were used. Case number (n), average survival time in months (m), 95% CI (shadow), long-rank P values, and hazard ratios are shown. *: P<0.05. ns: not significant.

FIG. 40A-40B demonstrates results from a Kaplan-Meier analysis and Cox univariate analysis in the Murat GBM dataset. Data were retrieved from GlioVis. Patients were divided into Cx43-high (red, top 25 percentile) and Cx43-low (blue, bottom 25 or 75 percentile) based upon Cx43 mRNA levels in primary, secondary, and recurrent GBM (All GBM), primary GBM only (Primary GBM), MGMT-deficient primary GBM (MGMT−), MGMT-expressing (MGMT+), or recurrent GBM only (Recurrent GBM). Kaplan-Meier analysis (FIG. 40A) and Cox univariate analysis (FIG. 40B) were used. Case number (n), average survival time in months (m), 95% CI (shadow), long-rank P values, and hazard ratios are shown. *: P<0.05. ns: not significant.

FIG. 41 demonstrates results from a Kaplan-Meier analysis in the CGGA recurrent GBM dataset. Data were retrieved from the CGGA data portal. Cx43-high (top 25 percentile) or Cx43-low (bottom 75 percentile) patients were divided into Radio (red, treated with radiation only) or Radio+chemo (blue, treated with radiation and chemotherapy) based on Cx43 mRNA levels in recurrent GBMs. Case number (n), average survival time in months (m), long-rank P values, and hazard ratios are shown. *: P<0.05 and ns: not significant.

FIG. 42A-42H demonstrates a correlation between connexins and PI3K catalytic subunits. Gene expression data were analyzed using the Pearson correlation coefficient assay. mRNA levels of Cx43 were compared to mRNA levels of PI3K catalytic subunits (FIGS. 42A, 42C, 42E, and 42G) in four different datasets as indicated. mRNA levels of PIK3CB were compared to those of connexin mRNAs (FIGS. 42B, 42D, 2F, and 42H). The coefficient r and corresponding P values are shown.

FIG. 43A-43D demonstrates optimization of αCT1, TGX-221 and TMZ in U87MG cells. (FIG. 43A) Combination of 20 μM TGX-221 and TMZ at various concentrations. U87MG cells were treated with drug combinations as indicated for 6 days. Cell viability was determined using the MTS viability assay. The vehicle DMSO was the control and set as 100%. Treated cells were normalized to DMSO-treated cells. (FIG. 43B) Combination of 50 μM TMZ and TGX-221 at various concentrations. (FIG. 43C) Combination of 20 μM TGX-221/50 μM TMZ and αCT1 at different concentrations. (FIG. 43D) Scores of Excess Over Bliss in FIG. 43C calculated using the Bliss Independence model. One-way ANOVA and student t test were used to determine statistical significance.

FIG. 44A-44C demonstrates the effect of an αCT1/TGX combo in VTC-003 and VTC-005. (FIG. 44A) Viability of VTC-003 cells treated with different drug combinations for 6 days. Cell viability was determined using the MTS viability assay. (FIG. 44B) Viability of VTC-005 cells treated with different drug combinations. (FIG. 44C) Scores of Excess Over Bliss calculated using the Bliss Independence model. One-way ANOVA or student t test were used to determine statistical significance.

FIG. 45A-45B demonstrates the effect of the αCT1/TGX combo in A172. (FIG. 45A) Viability of A172 cells treated with different drug combinations for 6 days. Cell viability was determined using the MTS viability assay. (FIG. 45B) Scores of Excess Over Bliss calculated using the Bliss Independence model. One-way ANOVA or student t test were used to determine statistical significance.

FIG. 46A-46E demonstrates that a combination of αCT1 and GSK2636771 overcomes TMZ resistance. (FIG. 46A) The effect of the αCT1/GSK/TMZ combo in VTC-103 cells. Cells were treated with 50 μM TMZ, 25 μM GSK2636771, and/or 30 μM αCT1 including single agents, double combinations and the αCT1/GSK/TMZ combo. (FIG. 46B) The effect of the αCT1/GSK/TMZ combo in LN229 cells. (FIG. 46C) EOB scores of drug combinations in VTC-103 and LN229 cells. (FIG. 46D) The effect of the αCT1/TGX-221/TMZ or αCT1/GSK/TMZ combo in astrocytes. (FIG. 46E) EOB scores of drug combinations in astrocytes. One-way ANOVA with Dunnett test for correction of multiple comparisons or Student's t test was used to determine statistical significance. *: P<0.05; ns: not significant. Drug combinations with strong synergistic effect were marked in red.

FIG. 47A-47B demonstrates that the αCT1/GSK/TMZ combo overcomes TMZ resistance in U87MG cells.

FIGS. 48A-48D demonstrates that p110β is enriched in BRAFV600E/PTENlow melanoma and associated with PI3K activation and patient survival. Data were retrieved from DepMap, TCGA, and reference 82. Levels of PI3K proteins (FIGS. 48A and 48B) in melanoma are shown. (FIG. 48C) Pearson correlation coefficients between levels of p110α/p110β and pAKT-5473/pAKT-T308. (FIG. 48D) Cox analysis of TCGA melanoma using JMP. Hazard ratios >1 indicate more chance of death; Hazard ratios <1 refer to more chance of survival. T-test and one-way ANOVA with Bonferroni's multiple comparisons test were used for statistical analyses. ns: not significant; *: P<0.05; **: P<0.01; ****: P<0.0001.

FIG. 49A-49C demonstrates that p110βhyper melanoma cells are more sensitive to p110β inhibition. (FIG. 49A) Immunoblotting of p110s and signaling molecules. 3-actin (ACTB) was used as the loading control. (FIG. 49B) Summary of genetic mutations and protein levels in melanoma cells. Information regarding hot spot mutations was retrieved from e.g., DepMap. (FIG. 49C) Cells were treated with 0-40 μM of inhibitors. Cell viability was measured using CellTiter Blue. IC50s were calculated using Graphpad.

FIG. 50A-50B demonstrates that p110β deficiency inactivates PI3K. (FIG. 50A) p110βhyper brain tumor cells were transduced with viruses harboring Cas9 and gRNAs. p110 proteins and signaling molecules were measured using immunoblotting. (FIG. 50B) Melanoma cells were treated with DMSO or 10 μM AZD6482.

FIG. 51A-51I demonstrates that the β18 motif and Selectide-18. (FIG. 51A) 3D of a p110β/p85α complex determined by SWISS-MODEL and Cluspro. (FIG. 51B) Protein sequences were aligned using COBALT. C2 domains (top) are show. Red bars: same residues; Grey bars: different residues; Red lines: missing residues. TKKSTKTINPSKYQTIRK in p110β C2 (bottom) is shown and termed as 018. Docking of p110β and p85α (FIG. 51C) or p110α and p85α (FIG. 51D) was done by SWISS-MODEL, Cluspro, and Chimera. The 018 motif is labeled in magenta. Deletion of β18 motif (FIG. 51E) changed the 3D shapes of p110β/p85 (FIG. 51C). (FIG. 51F) p110β cell-free kinase assay. An enzyme-linked immunosorbent assay (ELISA) was used to measure levels of lipids PIP2/PIP3. (FIG. 51G) Effect of PI3K inhibitors on p110s using ELISA. p110β (FIG. 51H) or p110α (FIG. 51I) was docked with Selectide-18-bound p85α using Cluspro/Chimera.

FIG. 52A-52F demonstrates that selectide-18 suppresses the growth of p110βhyper melanoma. (FIG. 52A) Cells were treated with 10 μM Selectide-18-FITC for 1 h and imaged immediately or after 96h. (FIG. 52B) Selectide-18-FITC in melanoma cells were imaged at different times. FITC was quantified using Image J and FITC intensities at different times were normalized with that at 1 h. Half-life was calculated using GraphPad. (FIG. 52C) UACC-62, MelST, or MeWo cells were treated with 50 μM of Selectide-18 for 4 days with daily repeated dosing. (FIG. 52D) MelST or melanoma cells were treated with 50 μM of Selectide-18 for 6 days with daily repeated dosing. Cell viability was measured using Cell-Titer Blue. (FIG. 52E) MelST, UACC-62, or MeWo cells were treated with Scramble or Selectide-18 at different doses. IC50s were calculated using GraphPad. (FIG. 52F) Nod scid mice bearing UACC-62 subcutaneous tumors were treated with Selectide-18 (3 mg/kg) intratumoral injection/daily) for 16 days. Tumors were measured using a caliper and tumor volumes were calculated as (length×width2))/2. Student t test or ANOVA was used to determine P values. ns: not significant; **: P<0.01; ***P<0.001.

FIG. 53 shows a table demonstrating melanoma PDXs from the Wistar Institute.

FIG. 54A-54E demonstrates selectide-18 and its mutants. (FIG. 54A) In-silico analyses of Selectide-18 and its mutants. 3D structures of single residue deletions were modeled using the PEPFOLD and were then matchmade to that of wild type Selectide-18 to acquire RMSDs using ChimeraX. High RMSD indicates a discrete structure of mutant. Selectide-18 and its mutants were docked with p85α using DOCK. The binding affinity (kcal/mol) of mutants was normalized to that of wild type, yielding p85α-binding affinity (mutant/wild type). (FIG. 54B) 3D conformations of Selectide-18 and its mutants. (FIG. 54C) Docking of Selectide-18 and its mutants on p85α. Hydrogen bonds (H-bonds)<3.3 Å are shown. (FIG. 54D) Docking of Selectide-18 and its mutants on p110/p85α. p85α was pre-docked with Selectide-18 or its mutants using DOCK. Resulting structures were docked with p110β using Cluspro. One representative model is shown. (FIG. 54E) Distances between p110β (ARG557, GLU546, ARG481) and p85α (ASN421, LYS424, and TYR425) were measured using ChimeraX. One-way ANOVA and t-test were used. ns: not significant; ****: P<0.0001.

FIG. 55 shows the 3D culture of tumor cells.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’, where ‘x’ and ‘y’ are numerical values, includes “about ‘x’ to about ‘y’.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

As used herein, “additive effect” refers to an effect arising between two or more molecules, compounds, substances, factors, or compositions that is equal to or the same as the sum of their individual effects.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “aptamer” can refer to single-stranded DNA or RNA molecules that can bind to pre-selected targets including proteins with high affinity and specificity. Their specificity and characteristics are not directly determined by their primary sequence, but instead by their tertiary structure.

As used herein, “cell type” refers to the more permanent aspects (e.g. a hepatocyte typically can't on its own turn into a neuron) of a cell's identity. Cell type can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160.

As used herein, “chemotherapeutic agent” or “chemotherapeutic” refers to a therapeutic agent utilized to prevent or treat cancer.

As used herein, “control” can refer to an alternative subject or sample used in an experiment for comparison purpose and included to minimize or distinguish the effect of variables other than an independent variable.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, the terms “disease” or “disorder” are used interchangeably throughout this specification, and refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition, or affliction.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

As used herein, “mammal,” for the purposes of treatments, can refer to any animal classified as a mammal, including human, domestic and farm animals, nonhuman primates, and zoo, sports, or pet animals, such as, but not limited to, dogs, horses, cats, and cows.

As used herein, “marker” is a term of art and commonly broadly denotes a biological molecule, more particularly an endogenous biological molecule, and/or a detectable portion thereof, whose qualitative and/or quantitative evaluation in a tested object (e.g., in or on a cell, cell population, tissue, organ, or organism, e.g., in a biological sample of a subject) is predictive or informative with respect to one or more aspects of the tested object's phenotype and/or genotype. The terms “marker” and “biomarker” may be used interchangeably throughout this specification.

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein “reduced expression” or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, object surface, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a PI3K mediated disease, which are further described in greater detail elsewhere herein. The effect can be therapeutic in terms of a partial or complete cure of a disease, condition, symptom or adverse effect attributed to the disease, disorder, or condition. The term “treatment” as used herein covers any treatment of a PI3K mediated disease, in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein “tumor” or “tumor tissue” refer to an abnormal mass of tissue resulting from excessive cell division. A tumor or tumor tissue comprises “tumor cells” which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein in the context of polynucleotides and polypeptides, “variant” can refer to a polynucleotide or polypeptide that differs from a reference polynucleotide or polypeptide, but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Despite significant efforts and advancements cancer still remains a main health problem leading to significant mortality and morbidity. Recurrence and chemoresistance of cancers are key roadblocks in the development of effective treatments, particularly for some cancers such as glioblastoma and cutaneous melanoma. Indeed, GBM accounts for 55% of all malignant brain cancers. Despite maximal debulking of the tumor and combinational treatment with radiation and chemo drugs, the 5-year overall survival is still agonizingly 5%. This poor prognosis is largely attributed to the high incidence of tumor progression such as recurrence. Progressive GBMs are characterized by highly infiltrative and tumorigenic GBM cells or GBM stem cells (GSCs). GSCs have a strong capability to grow tumors in mice and are resistant to radiation and chemotherapies, thereby becoming the culprit of tumor recurrence and progression.

Likewise, cutaneous melanoma is a difficult-to-treat cancer. While the 5-year survival rate of localized melanoma is about 99%, this rate drops to 65% or 25% when the tumor becomes regional or distant. Somatic mutations are frequently found in BRAF (50-60%), NRAS (15-30%), NF1 (14%), and PTEN (8˜10%), yielding four molecular subtypes: BRAF, RAS, NF1, and triple wild-type. Amongst all BRAF mutations, BRAF^{V600E} accounts for 75 to 90% of cases. BRAF^{V600E}-specific inhibitors and MEK inhibitors have achieved promising clinical outcomes. However, drug resistance and tumor progression occur inevitably, manifested by the unresponsiveness to these treatments in about 15% of BRAF^{V600E} melanoma as well as de novo or acquired resistance.

In both GBM and cutaneous melanoma, recurrence and/or chemoresistance is due, at least in part, to the activation of the phosphoinositide 3-kinase (PI3K) pathway. Although PI3K has been a target of recent therapeutic developments, such efforts have not yet translated into a clinically relevant therapeutic. PI3K has four catalytic subunits PIK3CA, PIK3CB, PIK3CD, and PIK3CG (PI3K catalytic subunit α, β, δ, and γ) that encode p110α, β, δ, and γ (also called PI3Kα, β, δ, and γ), respectively. PI3K catalytic subunits form a signaling complex with PI3K regulatory subunits PIK3R1, PIK3R2, or PIK3R3 that encodes p85α, p85β, or p55γ, respectively. In at least the case of melanoma mutations in PI3K genes have been reported in drug-naïve and -resistant melanoma. But the mutation rate is low (<1%). It is well-documented that PI3K activation causes therapy resistance. The current efforts to overcome chemoresistance by inhibiting PI3K have failed partially because pan-PI3K drugs that block all PI3K subunits are highly toxic. Further, the incomplete and sometimes contradictory understanding of the contribution of the various PI3K catalytic subunits has impaired the development of selective therapeutics effective for treating cancers, particularly treatment refractory cancers.

Additionally, PI3K activation can be stimulated by various activities of other molecules. For example, in at least the case of GBM, gap junction protein connexin 43 (Cx43) renders glioblastoma resistant to chemotherapy by non-channel forming activities including binding of the p110beta/p85 signaling complex upon receiving signals from extracellular cues by the carboxy terminus of Cx43. This selective binding brings the p110beta/p85 signaling complex to the membrane and subsequently activates AKT. Activated PI3K/AKT signaling renders GBM cells resistant to TMZ, which is independent of MGMT. This previously unappreciated activity of Cx43 has also contributed to the failure of development of effective approaches to treating cancers such as GBM, particularly chemoresistant cancers.

With that said, embodiments disclosed herein can provide compositions, formulations, and methods for treating cancers, particularly those whose pathogenesis, chemoresistance, and/or recurrence are due at least in part to PI3K, overexpression and/or activation, mediated particularly via the p110beta subunit and/or Cx43. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### P110Beta Selective Compositions

Described in several embodiments herein are engineered peptides that can include or be composed entirely and only of a p110beta targeting peptide; and a delivery moiety, where the delivery moiety is coupled to the p110beta targeting peptide. “Coupled to” as used in this context herein, refers to coupling via direct fusion at the C-terminus and/or N-terminus of the p110beta targeting peptide or indirectly via an intervening linker molecule(s) (peptide or other linkage) to the C-terminus and/or N-terminus or at one or more internal amino acids, such as via an ester or other suitable linkage to the one or more internal amino acids. Such linkers are generally known in the art and include, without limitation, Gly-Ser linkers. Any method of peptide synthesis and or generation can be used to make the engineered peptide. Such techniques are generally known in the art. In some embodiments, the delivery moiety is also a targeting moiety. In some embodiments, the delivery moiety is not a targeting moiety. In some embodiments, in addition to the delivery moiety (whether it be a targeting moiety or not), the engineered peptide includes one or more targeting moieties.

Also described herein are nucleic acids and vectors capable of expressing said engineered peptides.

### Engineered Peptides

p110beta Targeting Peptides

The engineered peptide includes a p110beta targeting peptide. In some embodiments, the p110beta targeting peptide is capable of selectively binding p110beta or a complex thereof, selectively inhibiting p110beta activity, or both. In some embodiments, the p110beta targeting peptide at least in part or in whole is composed of a peptide motif unique to the p110beta catalytic subunit of PI3K.

In some embodiments, the p110beta targeting peptide includes or is composed entirely of an amino acid sequence that is about 95 to 100 percent identical to SEQ ID NO: 3 or is a homologue thereof, or functional variant thereof. In some embodiments, the p110beta targeting peptide includes or is composed entirely of an amino acid sequence that is about 95, 96, 97, 98, 99, 99.1, 99.2, 99.3, 99.5, 99.5, 99.6, 99.7, 99.8, 99.9 or 100 percent identical to SEQ ID NO: 3 or is a homologue thereof, or functional variant thereof.

In some embodiments, the p110beta targeting peptide is modified, such as with one or more chemical or amino acid modifications, to alter its properties (e.g., cell uptake, stability, modified half-life (increased or decreased), etc.). In some embodiments, one or more amino acids are included in the D configuration as opposed to the L configuration, which can increase resistance to degradation. In some embodiments, one or more lysines present in the cell penetrating peptide are replaced with ornithine, which can improve resistance to degradation. In some embodiments, delivery efficiency can be improved by including modifications to the structure of the peptides into dendrimers or cyclization. In some embodiments, the modification includes phosphorylating one or more of the residues and/or adding one or more hydrophobic stearyl-moieties can be added to the peptide, which can improve pharmacokinetics and stability of the peptide in the bloodstream.

In some embodiments, one or more amino acids of the p110beta targeting peptide includes one or more ester linked groups. In some embodiments, the ester linkages of the ester linked groups are cleavable. The ester linked groups can, e.g., facilitate loading into a delivery vesicle, such as an exosome. For example, once loaded into a delivery vesicle such as an exosome, an esterase can cleave the cleavable ester group and trap the engineered peptide in the delivery vesicle such as an exosome. This is further described in International Patent Application Publication WO 2020/028439.

### Delivery Moieties

As previously described, the engineered peptide can include one or more delivery moieties. The delivery moieties(s) can facilitate, for example, one or more activities involved with delivery of the engineered peptide, including but not limited to, cell targeting, uptake, crossing membranes (such as cell membrane, nuclear membranes, or tissue membranes (e.g., the blood brain barrier), or loading into a vesicle, or any combination thereof.

Delivery moieties include, without limitation, polypeptides, peptides, oligonucleotides, polynucleotides, small chemical molecules, and combinations thereof. Delivery moieties can be antibodies or fragments thereof, receptors for e.g., surface ligands, receptor ligands (e.g., cell or other membrane surface receptor ligands), aptamers, and the like.

In some embodiments, the delivery moiety is a cell penetrating peptide. In some embodiments, the cell penetrating peptide is cationic. In some embodiments, the cell penetrating peptide is anionic. In some embodiments, the cell penetrating peptide is neutral. In some embodiments, the cell penetrating peptide is amphipathic. In some embodiments, the cell penetrating peptide is hydrophobic. In some embodiments, the cell penetrating peptide has a sequence identical to SEQ ID NO: 4, SEQ ID NO: 6, any one of SEQ ID NOs.: 110-222, and any of those set forth in Borrelli et al. 2018. Molecules. 23, 295 (pp. 1-28), which is incorporated by reference as if expressed in its entirety herein, particularly at Table 1 therein.

In some embodiments, the cell penetrating peptide is modified, such as with one or more chemical or amino acid modifications, to alter its properties (e.g., cell uptake, stability, modified half-life (increased or decreased), etc.). In some embodiments, one or more amino acids are included in the D configuration as opposed to the L configuration, which can increase resistance to degradation. In some embodiments, one or more lysines present in the cell penetrating peptide are replaced with ornithine, which can improve resistance to degradation. In some embodiments, delivery efficiency can be improved by including modifications to the structure of the peptides into dendrimers or cyclization. In some embodiments, the modification includes phosphorylating one or more of the residues and/or adding one or more hydrophobic stearyl-moieties can be added to the peptide, which can improve pharmacokinetics and stability of the peptide in the bloodstream. In some embodiments, the peptide is such that it can facilitate targeting to specific cell types and/or cell compartments (e.g., cytoplasm, nucleus, etc.,) and/or specific endocytic pathways.

The cell penetrating peptide can facilitate delivery of the p110beta targeting peptide to a cell by facilitation uptake by a cell via direct penetration and/or endocytosis (including micropinocytosis, clathrin-mediated endocytosis, and/or caveolin-mediated endocytosis). The cell penetrating peptide can also, in some embodiments, facilitate escape from endosomes.

Other functionalities of the cell penetrating peptide will be appreciated (see also e.g., Borrelli et al. 2018. Molecules. 23, 295 (pp. 1-28).

In some embodiments, one or more amino acids of the cell penetrating peptide includes one or more ester linked groups. In some embodiments, the ester linkages of the ester linked groups are cleavable. The ester linked groups can, e.g., facilitate loading into a delivery vesicle, such as an exosome. For example, once loaded into a delivery vesicle such as an exosome, an esterase can cleave the cleavable ester group and trap the engineered peptide in the delivery vesicle such as an exosome. This is further described in International Patent Application Publication WO 2020/028439.

### Other Functional Components

The engineered peptide can further include one or more other functional components, such as detection labels, purification tags, imaging labels, and the like. Such additional components can be biologic molecules (e.g., peptides, polypeptides, oligonucleotides, and polynucleotides), radioisotopes and radioscopes (e.g., ¹⁴C, ³H, ³⁵S, ³²P, Technetium-99m, radioiodine, and others). Detection labels include dyes (e.g., visible, fluorescent, infrared, UV, etc.), radiolabels, and/or the like. Purification and/or detection tags include, but are not limited to, synthetic and natural epitope tags, such as His-tags, FLAG-tags, HA-tags, GST-tags, MYC-tag, V5 tags, and/or the like. The additional function component(s) can be coupled to the N-terminus, and/or C-terminus and/or to an internal amino acid residue.

### Nucleic Acids and Vectors Encoding the Engineered Peptides

Also described in several embodiments herein are polynucleotides and vectors capable of encoding or that encode one or more of the engineered peptides. In general, the polynucleotides and vectors can be used to produce the engineered proteins, in vitro, in vivo, and/or ex vivo to a subject to which the engineered peptide can be administered. In some embodiments and as described in greater detail elsewhere herein the engineered peptide is administered to a subject for treatment of a disease, such as a PIK3 disease. In other embodiments, a polynucleotide such as DNA or RNA encoding the engineered peptide is administered to a subject such that the engineered peptide will be produced in the subject from the encoding polynucleotide.

In some embodiments, the polynucleotide can be codon optimized.

In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a engineered peptide or protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The polynucleotide can be codon optimized for expression in a specific cell-type, tissue type, organ type, and/or subject type. In some embodiments, a codon optimized sequence is a sequence optimized for expression in a eukaryote, e.g., humans (i.e. being optimized for expression in a human or human cell), or for another eukaryote, such as another animal (e.g. a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a polynucleotide coding sequence encoding one or more engineered peptide encoding polynucleotides described herein is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate.

### Vectors

Also provided herein are vectors that can contain one or more of the engineered peptide encoding polynucleotides described herein. The vectors can be useful in producing bacterial cells, fungal cells, yeast cells, plant cells, animal cells, and transgenic animals that can express one or more engineered peptides described herein. Such cells and/or organisms can be used for production of the engineered peptides. Within the scope of this disclosure are vectors containing one or more of the polynucleotide sequences described herein. One or more of the polynucleotides encoding the engineered peptides described herein can be included in a vector or vector system. The vectors and/or vector systems can be used, for example, to express one or more of the polynucleotides in a cell, such as a producer cell, to produce viral particles for transduction of other cells with polynucleotides encoding the engineered peptides described elsewhere herein. Other uses for the vectors and vector systems described herein are also within the scope of this disclosure, such as assays, in vitro peptide production, and the like. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other aspects of the vectors and vector systems are described elsewhere herein.

### Cell-Based Vector Amplification and Expression

Vectors can be designed for expression of engineered peptides described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include but are not limited to Sf9 and Sf21. In some embodiments the host cell is a suitable yeast cell. In some embodiments the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U20S, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2 plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other aspects can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of the engineered peptide encoding polynucleotides so as to drive expression of the one or more engineered peptides described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In aspects, the polynucleotides and/or vectors thereof described herein can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the 3-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit P-globin (Proc. Natl. Acad. Sci. USA, Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments, a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8al (Ncxl)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Ferl14), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the polynucleotides encoding the engineered peptides described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the polynucleotides encoding an engineered peptide described elsewhere herein components are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in expression of a polynucleotide encoding one or more engineered peptides described elsewhere herein are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include, but is not limited to, sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more polynucleotides encoding one or more engineered peptides described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g., aspects of inducible DNA binding proteins and methods ofuse and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector or system thereof can include one or more elements capable of translocating and/or expressing a polynucleotide encoding one or more engineered pareptides described elsewhere herein to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc.

### Selectable Markers and Tags

One or more of the polynucleotides encoding an engineered peptide operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered peptide encoding polynucleotide such that the selectable marker polypeptide, when translated, is inserted between two amino acids between the N- and C-terminus of the engineered peptide or at the N- and/or C-terminus of the engineered peptides. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more engineered peptides described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as P-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more polynucleotides encoding an engineered peptide and/or engineered peptide described herein via suitable linker, such as a glycine or glycine serine linkers, which are generally known in the art. Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the engineered peptide encoding polynucleotide(s) and/or products expressed therefrom (e.g., engineered peptides) include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated engineered peptide encoding polynucleotide(s) to specific cells, tissues, organs, etc.

### Cell-Free Vector and Polynucleotide Expression

In some embodiments, the polynucleotide encoding one or more engineered peptides can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Cx43 Targeting Compositions

Also described herein are Cx43 targeting compositions, and more specifically, Cx43 inhibiting compositions (e.g., Cx43 inhibitors). The pharmaceutical formulation of claim 43, wherein the Cx43 inhibitor is a biologic molecule-based inhibitor, a chemical molecule inhibitor, a small molecule inhibitor, an RNAi-based inhibitor, or a genetic modifier-based inhibitor. As used in this context herein “biologic molecule-based”, refers to a compound that is based in part or in whole on a biologic molecule such as DNA, RNA, peptides, polypeptides and the like. For example, in some embodiments a biologic molecule-based Cx43 inhibitor can be an RNAi polynucleotide (e.g., an antisense oligo, short hairpin RNA, siRNA, etc.) that can mediate a decrease or elimination of detectable Cx43 mRNA. In some embodiments, the Cx43 inhibitor is a DNA aptamer specific for Cx43. In some embodiments, the Cx43 inhibitor is an antibody or fragment thereof specific for Cx43.

As used herein, “genetic modifier-based inhibitor” refers to a genetic modifier engineered to modify a gene encoding Cx43 such that expression of the Cx43 mRNA and/or protein is altered such that a function of the Cx43 is decreased or eliminated (such as binding or otherwise activating PIK3). Exemplary genetic modifiers for the genetic modifier-based inhibitor for Cx43 include, but are not limited to, CRISPR-Cas systems, TALENs, Meganucleases, Prime-editing systems, RNA-editing systems, and the like. Such systems are generally known in the art and can be readily modified to target Cx43 using methods known in the art.

In some embodiments, the connexin 43 inhibitor is a peptide selected from the group of aCT1 (SEQ ID NO: 7), aCT11 (SEQ ID NO: 8), aCT1 minus I (SEQ ID NO: 9), aCT11 minus I (SEQ ID NO: 10), a peptide including an amino acid sequence identical to any one of SEQ ID NOs: 11-109, or any combination thereof. In some embodiments, peptide includes one or more ester-linked groups. In some embodiments, the ester linkages of the ester linked groups are cleavable. The ester linked groups can, e.g., facilitate loading into a delivery vesicle, such as an exosome. For example, once loaded into a delivery vesicle such as an exosome, an esterase can cleave the cleavable ester group and trap the engineered peptide in the delivery vesicle such as an exosome. This is further described in International Patent Application Publication WO 2020/028439.

The Cx43 inhibitor peptide can include one or more delivery moieties. The delivery moieties(s) can facilitate, for example, one or more activities involved with delivery of the Cx43 inhibitor peptide, including but not limited to, cell targeting, uptake, crossing membranes (such as cell membrane, nuclear membranes, or tissue membranes (e.g., the blood brain barrier), or loading into a vesicle, or any combination thereof.

Delivery moieties include, without limitation, polypeptides, peptides, oligonucleotides, polynucleotides, small chemical molecules, and combinations thereof. Delivery moieties can be antibodies or fragments thereof, receptors for e.g., surface ligands, receptor ligands (e.g., cell or other membrane surface receptor ligands), aptamers, and the like.

In some embodiments, the delivery moiety is a cell penetrating peptide. In some embodiments, the cell penetrating peptide is cationic. In some embodiments, the cell penetrating peptide is anionic. In some embodiments, the cell penetrating peptide is neutral. In some embodiments, the cell penetrating peptide is amphipathic. In some embodiments, the cell penetrating peptide is hydrophobic. In some embodiments, the cell penetrating peptide has a sequence identical to SEQ ID NO: 4, SEQ ID NO: 6, any one of SEQ ID NOs.: 110-222, and any of those set forth in Borrelli et al. 2018. Molecules. 23, 295 (pp. 1-28), which is incorporated by reference as if expressed in its entirety herein, particularly at Table 1 therein.

In some embodiments, the Cx43 inhibitor peptide (including any peptide moiety included (e.g., a cell penetrating peptide) is modified, such as with one or more chemical or amino acid modifications, to alter its properties (e.g., cell uptake, stability, modified half-life (increased or decreased), etc.). In some embodiments, one or more amino acids are included in the D configuration as opposed to the L configuration, which can increase resistance to degradation. In some embodiments, one or more lysines present in the Cx43 inhibitor peptide are replaced with ornithine, which can improve resistance to degradation. In some embodiments, delivery efficiency can be improved by including modifications to the structure of the peptides into dendrimers or cyclization. In some embodiments, the modification includes phosphorylating one or more of the residues and/or adding one or more hydrophobic stearyl-moieties can be added to the peptide, which can improve pharmacokinetics and stability of the peptide in the bloodstream. In some embodiments, the peptide is such that it can facilitate targeting to specific cell types and/or cell compartments (e.g., cytoplasm, nucleus, etc.,) and/or specific endocytic pathways.

A cell penetrating peptide can facilitate delivery of the Cx43 inhibitor peptide to a cell by facilitation uptake by a cell via direct penetration and/or endocytosis (including micropinocytosis, clathrin-mediated endocytosis, and/or caveolin-mediated endocytosis). The cell penetrating peptide can also, in some embodiments, facilitate escape from endosomes.

Other functionalities of the cell penetrating peptide will be appreciated (see also e.g., Borrelli et al. 2018. Molecules. 23, 295 (pp. 1-28).

The Cx43 inhibitor peptide can further include one or more other functional components, such as detection labels, purification tags, imaging labels, and the like. Such additional components can be biologic molecules (e.g., peptides, polypeptides, oligonucleotides, and polynucleotides), radioisotopes and radioscopes (e.g., ¹⁴C, ³H, ³⁵S, ³²P, Technetium-99m, radioiodine, and others). Detection labels include dyes (e.g., visible, fluorescent, infrared, UV, etc.), radiolabels, and/or the like. Purification and/or detection tags include, but are not limited to, synthetic and natural epitope tags, such as His-tags, FLAG-tags, HA-tags, GST-tags, MYC-tag, V5 tags, and/or the like. The additional function component(s) can be coupled to the N-terminus, and/or C-terminus and/or to an internal amino acid residue.

Any method of peptide synthesis and or generation can be used to make the Cx43 inhibitor peptide. Such techniques are generally known in the art.

### Delivery Vehicles

Also described in several exemplary embodiments herein are delivery vehicles that can include the p110Beta targeting peptide(s) and/or encoding polynucleotides and/or vectors, the Cx43 inhibitor(s), and/or other compositions, such as chemotherapeutics, other therapeutics, and other functional molecules. In some embodiments, the delivery vesicle is a single or bi-layered lipid or lipid membrane. In some embodiments, the delivery vehicle is a delivery vesicle. Exemplary delivery vesicles include, without limitation, micelles, liposomes, and exosomes. In some embodiments, the exosome is a milk exosome. Other suitable delivery vehicles are described in greater detail elsewhere herein

In some embodiments, the delivery vehicle includes an engineered peptide including ap110beta targeting peptide as described in greater detail elsewhere herein. In some embodiments, the delivery vehicle is a delivery vesicle. In some embodiments, the delivery vesicle is an exosome. In some embodiments the delivery vesicle, such as an exosome, further includes a connexin 43 inhibitor, a chemotherapeutic, an immune checkpoint inhibitor, or any combination thereof.

In some embodiments the delivery vehicle includes a connexin 43 inhibitor; chemotherapeutic; and optionally a PI3K inhibitor.

In some embodiments, the Cx43 inhibitor is a biologic molecule-based inhibitor, a chemical molecule inhibitor, a small molecule inhibitor, an RNAi-based inhibitor, or a genetic modifier-based inhibitor. In some embodiments, the connexin 43 inhibitor is a peptide selected from the group consisting of: αCT1 (SEQ ID NO: 7), αCT11 (SEQ ID NO: 8), αCT1 minus I (SEQ ID NO: 9), αCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In some embodiments, the PI3K inhibitor is a selective p110beta inhibitor. In some embodiments, the selective p110beta inhibitor is a biologic molecule-based inhibitor, a chemical molecule inhibitor, a small molecule inhibitor, an RNAi-based inhibitor, or a genetic modifier-based inhibitor.

In some embodiments, the selective p110beta inhibitor is an engineered peptide including a p110beta targeting peptide and a delivery moiety, wherein the delivery moiety is coupled to the p110beta targeting peptide, and wherein the engineered peptide optionally comprises one or more ester-linked groups. Such engineered peptides are described in greater detail elsewhere herein. In some embodiments, the p110beta targeting peptide is capable of selectively binding p110beta or a complex thereof, selectively inhibiting p110beta activity, or both. In some embodiments, the p110beta targeting peptide is composed entirely of or includes an amino acid sequence that is about 95 to 100 identical to SEQ ID NO: 3 or is a homologue thereof, or functional variant thereof. In some embodiments, the delivery moiety is a cell penetrating peptide. In some embodiments the cell penetrating peptide has a sequence identical to SEQ ID NO: 4, SEQ ID NO: 6, or any one of SEQ ID NOs: 110-222. Other suitable delivery moieties are described elsewhere herein with respect to the engineered peptide.

In some embodiments, the PI3K inhibitor is a biologic molecule-based inhibitor, a chemical molecule inhibitor, a small molecule inhibitor, an RNAi-based inhibitor, or a genetic modifier-based inhibitor.

In some embodiments the immune checkpoint inhibitor is a biologic molecule-based inhibitor, a chemical molecule inhibitor, a small molecule inhibitor, an RNAi-based inhibitor, or a genetic modifier-based inhibitor. The immune check point inhibitor can act to modify expression and/or activity of PDL-1, CTLA-4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCYlA2, GUCYlA3, GUCYlB2, GUCYlB3, MT1, MT2, CD40, OX40, CD137, GITR, CD27, SUP-1, TIM-3, CEACAM-1, CEACAM-3, or CEACAM-5. Other targets will be known in the art. In some embodiments the immune checkpoint inhibitor is a biologic molecule-based inhibitor, a chemical molecule inhibitor, a small molecule inhibitor, an RNAi-based inhibitor, or a genetic modifier-based inhibitor. In some embodiments, the immune checkpoint inhibitor is Pembrolizumab, Nivolumab, Cemiplimab, Atezlizumab, Aveumab, Durvalumab, Ipilimumab, and any combination thereof. See also Smith et al., 2019. Am. J. Transl. Res. 11(2): 529-541, Liu et al. 2021. Cancer Cell Int. 21. Article number 239, which are incorporated by reference in their entireties, for additional immune checkpoint inhibitors that can be included in the delivery vesicles herein.

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In some embodiments, the chemotherapeutic is not effective to treat a cancer or a chemotherapeutic resistant cancer having overexpression of p110beta when used alone. In some embodiments, the chemotherapeutic is not effective to treat a cancer or a chemotherapeutic resistant cancer having overexpression of p110beta, Cx43, or both when used alone. In some embodiments, the chemotherapeutic is temozolomide.

### Additional Exemplary Delivery Vehicles

The delivery vehicles may comprise non-viral vehicles. In general, methods and vehicles capable of delivering nucleic acids and/or proteins may be used for delivering the compositions, such as the engineered peptides and others described herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of the e.g., engineered peptides) and/or RNA molecules (e.g., mRNA encoding one or more of the engineered peptides).

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: 50168-3659(17)30038-X. doi: 10.1016/j.jconrel.2017.01.037. [Epub ahead of print]; Altmoglu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316;

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generated and/or deliver the e.g., engineered peptides described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the e.g., engineered peptides and/or encoding polynucleotides described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2b (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Ostergaard et al., Bioconjugate Chem., 2015, 26 (8), pp 1451-1455.

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin 33 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the e.g., engineered peptides and/or encoding polynucleotides described herein directly and delivered to cells.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the engineered peptide or other peptide or polypeptide encoding polynucleotide. A DNA nanoclew may be coated, e.g., coated with PEI to induce endosomal escape.

### Metal Nanoparticles

In some embodiments, the delivery vehicles comprise gold nanoparticles (also referred to AuNPs or colloidal gold). Gold nanoparticles may form complex with cargos, e.g., engineered peptides and/or encoding polynucleotides described herein. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US 20100129793.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA. Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January;267(1):9-21; El-Andaloussi S, et al., Nat Protoc. 2012 December; 7(12):2112-26; Uno Y, et al., Hum Gene Ther. 2011 June;22(6):711-9; Zou W, et al., Hum Gene Ther. 2011 April;22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/dObm00427h.

Other non-limiting, exemplary exosomes include any of those set forth in Alvarez-Erviti et al. 2011, Nat Biotechnol 29: 341; [1401] El-Andaloussi et al. (Nature Protocols 7:2112-2126(2012); and Wahlgren et al. (Nucleic Acids Research, 2012, Vol. 40, No. 17 e130).

As previously described the exosome can be a milk exosome. Milk exosomes can be obtained from any mammalian milk, including but not limited to, cow, camel, pig, goat, horse, sheep, non-human primate, and human.

### Spherical Nucleic Acids (SNAs)

In some embodiments, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Additional Cargos

In some embodiments, the delivery vehicle includes one or more other cargos such as additional compounds, including, but not limited to DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, genetic modifiers, radiation sensitizers, and any combination thereof.

Suitable hormones include, but are not limited to, amino-acid derived hormones (e.g., melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosterone, cortisol).

Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g., ibuprofen, naproxen, ketoprofen, and nimesulide), aspirin and related salicylates (e.g., choline salicylate, magnesium salicylate, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotonergic antidepressants (e.g. selective serotonin reuptake inhibitors, tricyclic antidepressants, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g. cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g. lomefloxacin, norfloxacin, ofloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable radiation sensitizers include, but are not limited to, 5-fluorouracil, platinum analogs (e.g. cisplatin, carboplatin, and oxaliplatin), gemcitabine, DNA topoisomerase I-targeting drugs (e.g. camptothecin derivatives (e.g. topotecan and irinotecan)), epidermal growth factor receptor blockade family agents (e.g. cetuximab, gefitinib), farnesyltransferase inhibitors (e.g., L-778-123), COX-2 inhibitors (e.g. rofecoxib, celecoxib, and etoricoxib), bFGF and VEGF targeting agents (e.g. bevazucimab and thalidomide), NBTXR3, Nimoral, trans sodium crocetinate, NVX-108, and combinations thereof. See also e.g., Kvols, L. K., J Nucl Med 2005; 46:187S-190S.

Suitable genetic modifiers include, but are not limited, CRISPR-Cas systems, TALENs, primer editors, mega nucleases, RNA base editing systems, and/or the like.

### Pharmaceutical Formulations

Also described herein are pharmaceutical formulations that can contain an amount, effective amount, and/or least effective amount, and/or therapeutically effective amount of one or more compounds, molecules, compositions, or a combination thereof (which are also referred to as the primary active agent or ingredient elsewhere herein) described in greater detail elsewhere herein a pharmaceutically acceptable carrier or excipient.

In some embodiments, the pharmaceutical formulation includes an engineered peptide that includes or is composed of a p110beta targeting peptide, which are described in greater detail elsewhere herein, and a pharmaceutically acceptable carrier. In some embodiments, the pharmaceutical formulation further includes a connexin 43 inhibitor, a chemotherapeutic; an immune checkpoint inhibitor; or any combination thereof.

In some embodiments, the pharmaceutical formulation includes a connexin 43 inhibitor; a chemotherapeutic; and optionally a PI3K inhibitor; and a pharmaceutically acceptable carrier.

In some embodiments, the Cx43 inhibitor is a biologic molecule-based inhibitor, a chemical molecule inhibitor, a small molecule inhibitor, an RNAi-based inhibitor, or a genetic modifier-based inhibitor. In some embodiments, the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In some embodiments, the PI3K inhibitor is a selective p110beta inhibitor. In some embodiments, the selective p110beta inhibitor is a biologic molecule-based inhibitor, a chemical molecule inhibitor, a small molecule inhibitor, an RNAi-based inhibitor, or a genetic modifier-based inhibitor.

In some embodiments, the selective p110beta inhibitor is an engineered peptide including a p110beta targeting peptide and a delivery moiety, wherein the delivery moiety is coupled to the p110beta targeting peptide, and wherein the engineered peptide optionally comprises one or more ester-linked groups. Such engineered peptides are described in greater detail elsewhere herein. In some embodiments, the p110beta targeting peptide is capable of selectively binding p110beta or a complex thereof, selectively inhibiting p110beta activity, or both. In some embodiments, the p110beta targeting peptide is composed entirely of or includes an amino acid sequence that is about 95 to 100 identical to SEQ ID NO: 3 or is a homologue thereof, or functional variant thereof. In some embodiments, the delivery moiety is a cell penetrating peptide. In some embodiments the cell penetrating peptide has a sequence identical to SEQ ID NO: 4, SEQ ID NO: 6, or any one of SEQ ID NOs: 110-222. Other suitable delivery moieties are described elsewhere herein with respect to the engineered peptide.

In some embodiments, the PI3K inhibitor is a biologic molecule-based inhibitor, a chemical molecule inhibitor, a small molecule inhibitor, an RNAi-based inhibitor, or a genetic modifier-based inhibitor.

In some embodiments the immune checkpoint inhibitor is a biologic molecule-based inhibitor, a chemical molecule inhibitor, a small molecule inhibitor, an RNAi-based inhibitor, or a genetic modifier-based inhibitor. The immune check point inhibitor can act to modify expression and/or activity of PDL-1, CTLA-4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCYlB3, MT1, MT2, CD40, OX40, CD137, GITR, CD27, SUP-1, TIM-3, CEACAM-1, CEACAM-3, or CEACAM-5. Other targets will be known in the art. In some embodiments the immune checkpoint inhibitor is a biologic molecule-based inhibitor, a chemical molecule inhibitor, a small molecule inhibitor, an RNAi-based inhibitor, or a genetic modifier-based inhibitor. In some embodiments, the immune checkpoint inhibitor is Pembrolizumab, Nivolumab, Cemiplimab, Atezlizumab, Aveumab, Durvalumab, Ipilimumab, and any combination thereof. See also Smith et al., 2019. Am. J. Transl. Res. 11(2): 529-541, Liu et al. 2021. Cancer Cell Int. 21. Article number 239, which are incorporated by reference in their entireties, for additional immune checkpoint inhibitors that can be included in the delivery vesicles herein.

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In some embodiments, the chemotherapeutic is not effective to treat a cancer or a chemotherapeutic resistant cancer having overexpression of p110beta when used alone. In some embodiments, the chemotherapeutic is not effective to treat a cancer or a chemotherapeutic resistant cancer having overexpression of p110beta, Cx43, or both when used alone. In some embodiments, the chemotherapeutic is temozolomide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions or any combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a PI3K disease or a symptom thereof. In some embodiments, the PI3K mediated disease is a cancer. In some embodiments, the PI3K mediated disease is a cancer, optionally a chemotherapy resistant cancer. In some embodiments, the cancer is characterized at least in part by overexpression of p110beta. In some embodiments, the cancer is characterized at least in part by overexpression of p110beta, Cx43, or both. In some embodiments, the cancer is glioblastoma or melanoma. In some embodiments the melanoma is cutaneous melanoma.

Other cancers include, but are not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including, but not limited to, Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, genetic modifiers, and any combination thereof.

Suitable hormones include, but are not limited to, amino-acid derived hormones (e.g., melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosterone, cortisol).

Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g., ibuprofen, naproxen, ketoprofen, and nimesulide), aspirin and related salicylates (e.g., choline salicylate, magnesium salicylate, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotonergic antidepressants (e.g. selective serotonin reuptake inhibitors, tricyclic antidepressants, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g. cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g. lomefloxacin, norfloxacin, ofloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable radiation sensitizers include, but are not limited to, 5-fluorouracil, platinum analogs (e.g. cisplatin, carboplatin, and oxaliplatin), gemcitabine, DNA topoisomerase I-targeting drugs (e.g. camptothecin derivatives (e.g. topotecan and irinotecan)), epidermal growth factor receptor blockade family agents (e.g. cetuximab, gefitinib), farnesyltransferase inhibitors (e.g., L-778-123), COX-2 inhibitors (e.g. rofecoxib, celecoxib, and etoricoxib), bFGF and VEGF targeting agents (e.g. bevazucimab and thalidomide), NBTXR3, Nimoral, trans sodium crocetinate, NVX-108, and combinations thereof. See also e.g., Kvols, L. K., J Nucl Med 2005; 46:187S-190S.

Suitable genetic modifiers include, but are not limited, CRISPR-Cas systems, TALENs, primer editors, mega nucleases, RNA base editing systems, and/or the like.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are reducing or eliminating PI3K expression and/or activity, reducing and/or eliminating one or more functions of Cx43, such as a non-channel forming activity of Cx43, reducing or eliminating Cx43 binding, activation, or other interaction with PI3K, particularly the p110beta catalytic subunit, reducing a cancer cell growth and/or viability, inducing cell death, particularly cancer cell death, reducing chemoresistance in a cancer cell, particularly a GBM or melanoma, or any combination thereof.

The effective amount, least effective amount, and/or therapeutically effective amount of the primary and optional secondary active agent described elsewhere herein contained in the pharmaceutical formulation can be any non-zero number ranging from about 0 to about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount, least effective amount, and/or therapeutically effective amount can be an effective concentration, least effective concentration, and/or therapeutically effective concentration, in which each can be any non-zero number ranging from about 0 to about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can be any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can any non-zero number ranging from about 0 to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

For example, in some embodiments, a co-therapy includes administration of a connexin 43 inhibitor, an amount of a chemotherapeutic, and an amount of a PI3K inhibitor in the same pharmaceutical formulation or as separate formulations that are administrated simultaneously or sequentially at different times. In other example embodiments, a co-therapy can include administration of an engineered peptide including a p110beta targeting peptide described herein and a chemotherapeutic, a Cx43 inhibitor, an immune checkpoint inhibitor or any combination thereof, where all are present in the same formulation or in separate formulations but are administered simultaneously or are administrated as separate formulations sequentially at different times.

### Kits

Any of the compounds, compositions, formulations, particles, cells, etc. described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof. In some embodiments, the subject in need thereof can be in need of treatment for a PI3K mediated disease. In some embodiments, the PI3K mediated disease is a cancer. In some embodiments, the PI3K mediated disease is a cancer, optionally a chemotherapy resistant cancer. In some embodiments, the cancer is characterized at least in part by overexpression of p110beta. In some embodiments, the cancer is characterized at least in part by overexpression of p110beta, Cx43, or both. In some embodiments, the cancer is glioblastoma or melanoma. In some embodiments the melanoma is cutaneous melanoma. In some embodiments, can indicate that one or more components of the kit have one or more therapeutic benefits or actions. In some embodiments, the one or more therapeutic effects are reducing or eliminating PI3K expression and/or activity, reducing and/or eliminating one or more functions of Cx43, such as a non-channel forming activity of Cx43, reducing or eliminating Cx43 binding, activation, or other interaction with PI3K, particularly the p110beta catalytic subunit, reducing a cancer cell growth and/or viability, inducing cell death, particularly cancer cell death, reducing chemoresistance in a cancer cell, particularly a GBM or melanoma, or any combination thereof.

Other cancers, which may be a PI3K mediated disease, include but are not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

### Methods of Treating a PI3K Mediated Disease

The compositions and formulations, such as pharmaceutical formulations and/or delivery vesicles described in greater detail elsewhere herein are methods of treating and/or preventing a PI3K mediated disease or a symptom thereof in a subject. In some embodiments, the PI3K mediated disease is a cancer. In some embodiments, the PI3K mediated disease is a cancer, optionally a chemotherapy resistant cancer. In some embodiments, the cancer is characterized at least in part by overexpression of p110beta. In some embodiments, the cancer is characterized at least in part by overexpression of p110beta, Cx43, or both. In some embodiments, the cancer is glioblastoma or melanoma. In some embodiments the melanoma is cutaneous melanoma. In some embodiments, can indicate that one or more components of the kit have one or more therapeutic benefits or actions. In some embodiments, the one or more therapeutic effects are reducing or eliminating PI3K expression and/or activity, reducing and/or eliminating one or more functions of Cx43, such as a non-channel forming activity of Cx43, reducing or eliminating Cx43 binding, activation, or other interaction with PI3K, particularly the p110beta catalytic subunit, reducing a cancer cell growth and/or viability, inducing cell death, particularly cancer cell death, reducing chemoresistance in a cancer cell, particularly a GBM or melanoma, or any combination thereof.

Other cancers, which may be a PI3K mediated disease, include but are not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

In some embodiments, the method of treating a PI3K mediated disease or a symptom thereof in a subject in need thereof includes administering an engineered peptide described elsewhere herein having a p110beta targeting peptide or being a p110beta targeting peptide. Exemplary engineered peptides are described in greater detail elsewhere herein. In some embodiments one or more of the engineered peptide having a p110beta targeting peptide or being a p110beta targeting peptide, the connexin 43 inhibitor; the chemotherapeutic; and/or the immune checkpoint inhibitor are administered simultaneously to the subject and/or one or more of the engineered peptide having a p110beta targeting peptide or being a p110beta targeting peptide, the connexin 43 inhibitor; the chemotherapeutic; and/or the immune checkpoint inhibitor are administered sequentially to the subject. In some embodiments, the method of treating a PI3K mediated disease or a symptom thereof in a subject in need thereof includes administering, to the subject in need thereof, a pharmaceutical formulation including an engineered peptide having a p110beta targeting peptide or being a p110beta targeting peptide and a pharmaceutically acceptable carrier. In some embodiments, the pharmaceutical formulation further includes a connexin 43 inhibitor; chemotherapeutic; an immune checkpoint inhibitor; or any combination thereof. In some embodiments, the method includes administering an engineered peptide having a p110beta targeting peptide or being a p110beta targeting peptide and a connexin 43 inhibitor; chemotherapeutic; an immune checkpoint inhibitor; or any combination thereof, to the subject. In some embodiments one or more of the engineered peptide having a p110beta targeting peptide or being a p110beta targeting peptide, the connexin 43 inhibitor; the chemotherapeutic; and/or the immune checkpoint inhibitor are administered simultaneously to the subject and/or one or more of the engineered peptide having a p110beta targeting peptide or being a p110beta targeting peptide, the connexin 43 inhibitor; the chemotherapeutic; and/or the immune checkpoint inhibitor are administered sequentially to the subject.

In some embodiments, the method of treating a PI3K mediated disease or a symptom thereof in a subject in need thereof includes administering a connexin 43 inhibitor; a chemotherapeutic; and optionally, a PI3K inhibitor to the subject in need thereof. In some embodiments, one or more of the connexin 43 inhibitor; a chemotherapeutic; and optionally, the PI3K inhibitor are delivered simultaneously to the subject and/or one or more of the connexin 43 inhibitor; the chemotherapeutic; and optionally, the PI3K inhibitor are delivered sequentially to the subject. In some embodiments, the method of treating a PI3K mediated disease or a symptom thereof in a subject in need thereof includes administering a pharmaceutical formulation including a connexin 43 inhibitor; a chemotherapeutic; and optionally, a PI3K inhibitor to the subject in need thereof.

In some embodiments, the method of treating a PI3K mediated disease or a symptom thereof in a subject in need thereof includes administering delivery vesicle(s), such as an exosome(s), (e.g., any of those described elsewhere herein, particularly under the heading “Delivery Vesicles” and the Working Examples herein) to the subject in need thereof.

In some embodiments, the Cx43 inhibitor is a biologic molecule-based inhibitor, a chemical molecule inhibitor, a small molecule inhibitor, an RNAi-based inhibitor, or a genetic modifier-based inhibitor. In some embodiments, the connexin 43 inhibitor is a peptide selected from the group consisting of: αCT1 (SEQ ID NO: 7), αCT11 (SEQ ID NO: 8), αCT1 minus I (SEQ ID NO: 9), αCT11 minus I (SEQ ID NO: 10), a peptide comprising an amino acid sequence identical to any one of SEQ ID NOs: 11-109, or any combination thereof, wherein the peptide optionally comprises one or more ester-linked groups.

In some embodiments, the PI3K inhibitor is a selective p110beta inhibitor. In some embodiments, the selective p110beta inhibitor is a biologic molecule-based inhibitor, a chemical molecule inhibitor, a small molecule inhibitor, an RNAi-based inhibitor, or a genetic modifier-based inhibitor. In some embodiments, the selective p110beta inhibitor is an engineered peptide including a p110beta targeting peptide and a delivery moiety, wherein the delivery moiety is coupled to the p110beta targeting peptide, and wherein the engineered peptide optionally comprises one or more ester-linked groups. Such engineered peptides are described in greater detail elsewhere herein. In some embodiments, the p110beta targeting peptide is capable of selectively binding p110beta or a complex thereof, selectively inhibiting p110beta activity, or both. In some embodiments, the p110beta targeting peptide is composed entirely of or includes an amino acid sequence that is about 95 to 100 identical to SEQ ID NO: 3 or is a homologue thereof, or functional variant thereof. In some embodiments, the delivery moiety is a cell penetrating peptide. In some embodiments the cell penetrating peptide has a sequence identical to SEQ ID NO: 4, SEQ ID NO: 6, or any one of SEQ ID NOs: 110-222. Other suitable delivery moieties are described elsewhere herein with respect to the engineered peptide.

In some embodiments, the PI3K inhibitor is a biologic molecule-based inhibitor, a chemical molecule inhibitor, a small molecule inhibitor, an RNAi-based inhibitor, or a genetic modifier-based inhibitor.

In some embodiments the immune checkpoint inhibitor is a biologic molecule-based inhibitor, a chemical molecule inhibitor, a small molecule inhibitor, an RNAi-based inhibitor, or a genetic modifier-based inhibitor. The immune check point inhibitor can act to modify expression and/or activity of PDL-1, CTLA-4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCYlB3, MT1, MT2, CD40, OX40, CD137, GITR, CD27, SUP-1, TIM-3, CEACAM-1, CEACAM-3, or CEACAM-5. Other targets will be known in the art. In some embodiments the immune checkpoint inhibitor is a biologic molecule-based inhibitor, a chemical molecule inhibitor, a small molecule inhibitor, an RNAi-based inhibitor, or a genetic modifier-based inhibitor. In some embodiments, the immune checkpoint inhibitor is Pembrolizumab, Nivolumab, Cemiplimab, Atezlizumab, Aveumab, Durvalumab, Ipilimumab, and any combination thereof. See also Smith et al., 2019. Am. J. Transl. Res. 11(2): 529-541, Liu et al. 2021. Cancer Cell Int. 21. Article number 239, which are incorporated by reference in their entireties, for additional immune checkpoint inhibitors that can be included in the delivery vesicles herein.

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In some embodiments, the chemotherapeutic is not effective to treat a cancer or a chemotherapeutic resistant cancer having overexpression of p110beta when used alone. In some embodiments, the chemotherapeutic is not effective to treat a cancer or a chemotherapeutic resistant cancer having overexpression of p110beta, Cx43, or both when used alone. In some embodiments, the chemotherapeutic is temozolomide.

In some embodiments of the method, the PI3K mediated disease is a cancer, optionally a chemotherapy resistant cancer. In some embodiments, the cancer is characterized at least in part by overexpression of p110beta, Cx43, or both. In some embodiments, the cancer is glioblastoma or melanoma.

In some embodiments of the previously described methods, the method includes delivering a polynucleotide and/or a vector encoding one or more of the engineered peptides and/or one or more Cx43 inhibitor peptides to a subject whereby the engineered peptide(s) and/or the Cx43 inhibitor peptide(s) are produced in the subject. In some embodiments, the encoding polynucleotide and/or vector that is delivered is a DNA or DNA vector. In some embodiments, the encoding polynucleotide that is delivered is and RNA, such as an mRNA.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Selectides for Glioblastoma

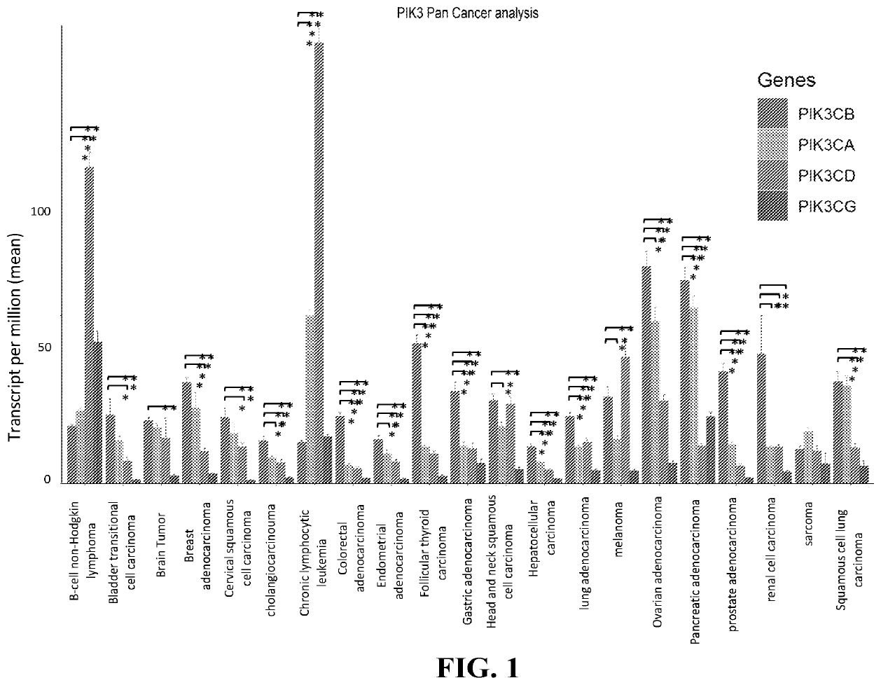

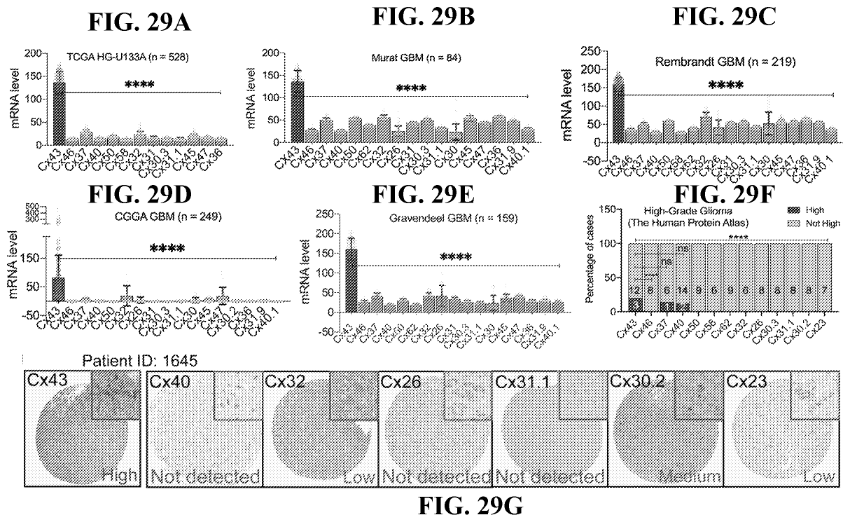

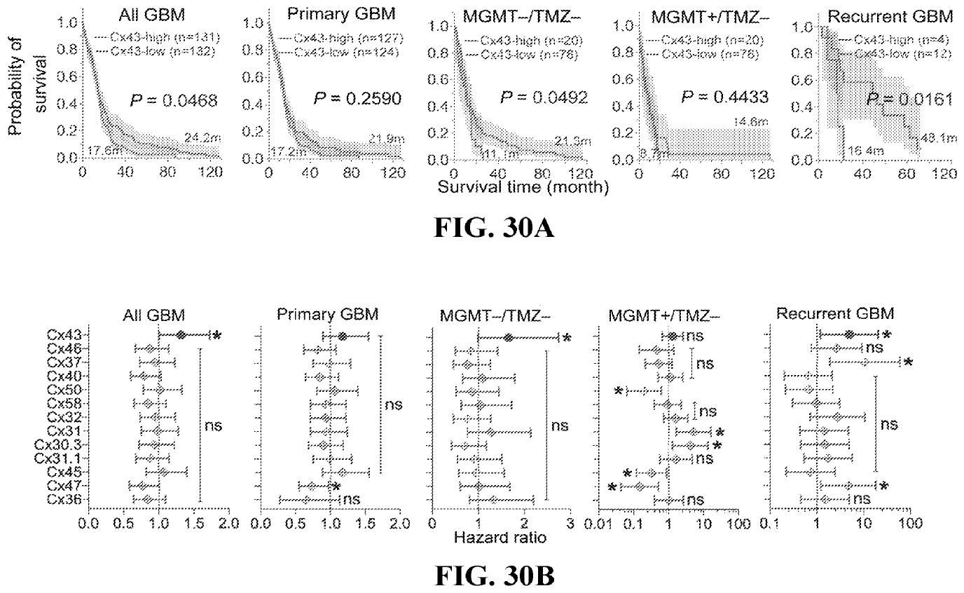

Aberrant PI3K expression and/or activation is present in various cancers (see e.g., FIG. 1). In mammals, there are four classes of PI3K (class IA, IB, II and III). Class IA PI3K is composed of three catalytic isoforms PIK3CA, PIK3CB, and PIK3CD (PI3K catalytic subunit α, β, and δ) that encode p110α, p, and S, respectively. These catalytic isoforms bind to one of the p85 regulatory isoforms α, β, or γ encoded by PIK3R1, PIK3R2, and PIK3R3 (PI3K regulatory subunit 1-3) to form a heterodimer. Class IB PI3K has the fourth catalytic isoform PIK3CG (encoding p110γ) that interacts with p101 encoded by PIK3R5. The p110γ/p101 complex is expressed in immune cells and barely detected in the brain. There are structural differences among the p110s (FIG. 25) and within the p110 C2 domains (FIG. 26).

PI3K binds to and converts membrane PIP₂ (phosphatidylinositol-4,5-biphosphate, PtdIns4,5P₂) into PIP₃ (PtdIns3,4,5P₃), the latter of which is a secondary messenger that activates downstream signaling pathways to promote GBM cell invasion and survival. For example, PI3K activates AKT serine/threonine kinase (AKT) and AKT inactivates forkhead box O3 (FOXO3)/BCL2 like 11 (BCL211, also known as BIM) to suppress apoptosis and promote cell survival. PI3K/AKT also activates rac family small GTPase 1 (RAC1) and matrix metallopeptidases to induce cell invasion.

Although several PI3K inhibitors are currently in development and use for treatment of cancer, they are largely ineffective for various reasons including, low selectivity and specificity as well as poor transport across the blood-brain barrier (relevant for brain and other CNS cancers, including, but not limited to, glioblastoma). Further complicating current cancer therapies is resistance to chemotherapeutics. This Example can at least demonstrate a peptide therapeutics (referred to as a “selectide”) that is capable of specifically inhibiting the expression and/or activity of PIK3CB (p110beta) subunit and its use to modify expression and/or activity in various cancer cells so as to treat a cancer, as exemplified by its effect on glioblastoma and melanoma.

As demonstrated in FIG. 1, the amount of PI3K subunit overexpression varies between cancers. Glioblastoma and melanoma are exemplary cancers that both have an increase in PI3KCB subunit overexpression (FIG. 1). Generally, prognosis of glioblastoma is poor, largely due to the high incidence of tumor progression such as recurrence. Progressive GBM cells are characterized by highly infiltrative and tumorigenic GBM cells or GBM stem cells. Progressive GBM cells are often refractory to current therapies and improved therapies targeting infiltrative GBM and GBM stem cells is warranted. Treatments of both can fail due to drug resistance and recurrence. PI3K signaling is important for survival of glioblastoma cells. Recent observations indicate that in glioblastoma a specific PI3K catalytic isoform PIK3CB/p110b (p110Beta) dominates in the activation of PI3K signaling and is more important than other subunits in promoting cell survival and proliferation. Mutations in PI3K genes have been reported in drug-naïve and -resistant melanoma, albeit with a low mutation rate (<1%). It is well known that PI3K activation causes therapy resistance in the case of melanoma, but overcoming this resistance through pan-PI3K inhibition has not translated to a clinically relevant therapy as previously mentioned. This failure is due in part because pan-PI3K drugs that block all PI3K subunits are highly toxic.

### Glioblastoma

### Divergent Roles of PI3K Isoforms in GBM

PIK3CB Presents the Strongest Correlation with GBM Recurrence/Progression.

To search for new biomarkers for GBMs, a short-hairpin RNA (shRNA)-based screen was used and 20 survival kinase genes were identified in GBM. Further analyses of nearly 500 GBM patients including 99 with recurrent tumors from The Cancer Genome Atlas database revealed that only PIK3CB showed the strongest correlation with high incidence, bolstered risk, and abysmal prognosis of progressive/recurrent GBMs, whereas other catalytic isoforms and regulatory subunits failed to do so. Mutations in PIK3CA and PTEN (phosphatase and tensin homolog, an inhibitor of PI3K pathway) were not associated with recurrence risk and patient prognosis. Applicant's prior research indicates that PIK3CB is a biomarker that defines a subset of GBM patients with high risk of tumor recurrence/progression that is independent of genetic alterations.

p110β is a Selective GBM Survival Factor

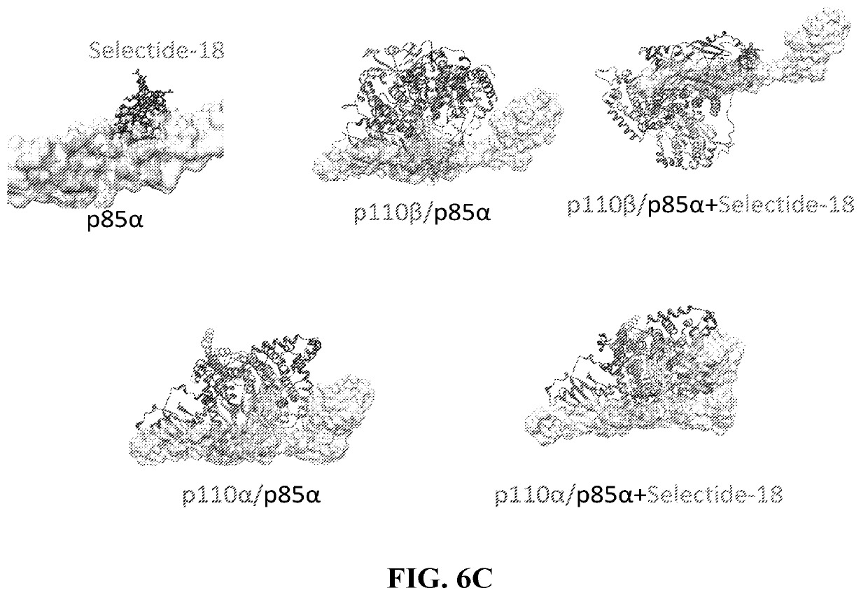

Applicants have demonstrated that: (1) Levels of p110β proteins (encoded by PIK3CB), but not other p110s, correlate with AKT activation; (2) Patients with high levels of p110β/AKT have significantly shorter life span; (3) Inhibition of p110β—but not p110α or p110δ-inactivates AKT in and suppresses the viability of GBM cell lines and primary GBM cells, while having no effect on astrocytes; (4) Importantly, endogenous p110α and p110δ fail to compensate the loss of p110β to re-activate AKT and rescue cells from cell death; (5) TGX-221 and GSK2636771 (p110β inhibitors) slow down tumor growth in mice; and (6) TGX-221 selectively blocks AKT activation, whereas the p110α inhibitor PIK-75 non-selectively inhibits AKT and ERK. Hence, p110β is a selective GBM survival factor. Our new and unpublished research further verified that knockout of p110β using CRISPR/Cas9 inactivated PI3K/AKT in p110βhigh SF295 cells (FIG. 7A), whereas deletion of p110α or p110δ had no effect on PI3K activity (FIGS. 7B-7C). Notably, ablation of p110β did not change levels of other PI3K isoforms and endogenous p110α or p110δ fails to substitute for p110β to reactivate AKT. Collectively, our results demonstrate that p110β is more important than other PI3K isoforms in AKT activation and GBM cell survival.

p110β Regulates GBM Cell Migration

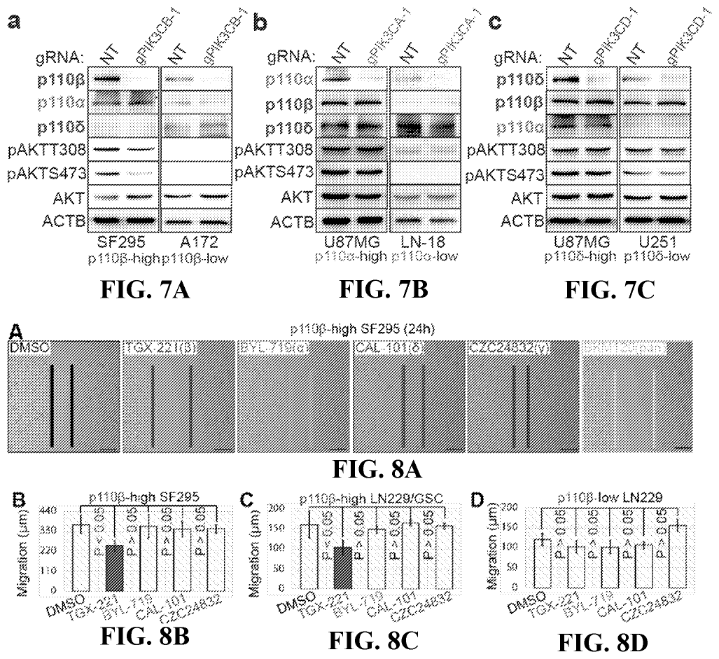

Invasion of GBM cells into surrounding brain tissues makes treating GBM difficult. PI3K signaling regulates cell migration and invasion in GBM; however, it remains unclear whether PI3K isoforms exhibit different activities. The wound healing assay showed that TGX-221 and BKM120 (a pan-PI3K inhibitor) inhibited the migration of p110βhigh SF295 cells and LN229/GSCs, whereas no change was found in cells treated with inhibitors of other PI3K isoforms (FIG. 8A-8C). p110 low LN229 cells did not respond to PI3K inhibitors (FIG. 8D). Hence, p110β is more important than other PI3K isoforms in regulating GBM cell migration.

Invasion and Apoptosis Signaling Pathways Downstream of p110β

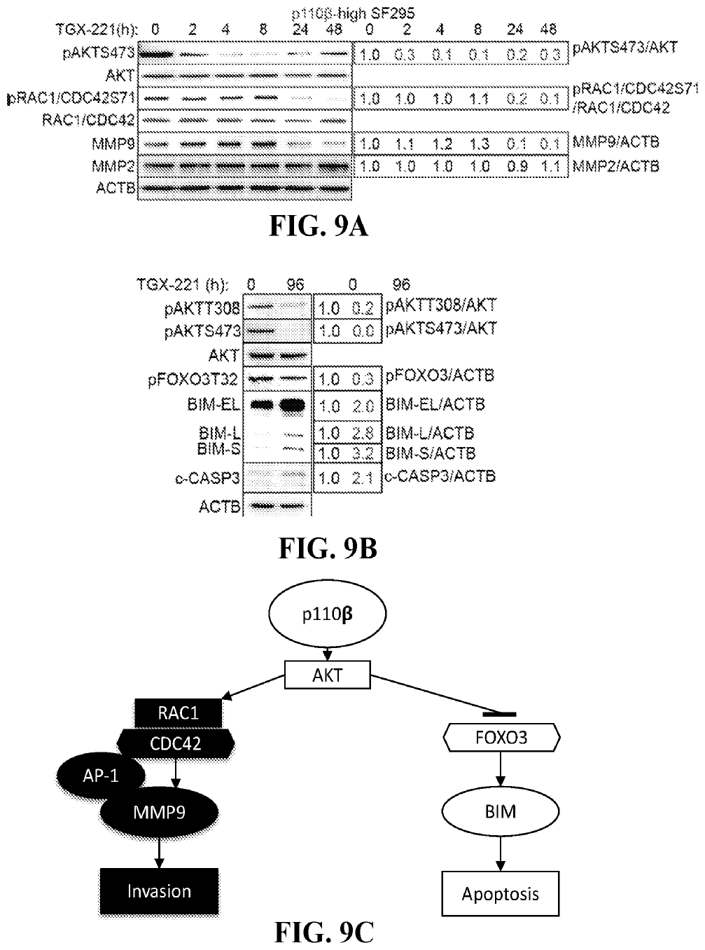

Prior research has revealed that the GTPases RAC1 and cell division cycle 42 (CDC42), activated by PI3K/AKT, induces expression of matrix metallopeptidases (i.e., MMP2 and MMP9) to regulate cell migration and invasion. However, whether p110β regulates RAC1/CDC42 and MMPs remains unknown. Applicant found that TGX-221, inactivated AKT and RAC1/CDC42 (phosphorylated by AKT) and decreased protein levels of 24-hour measurements with those of 0-hour. MMP9, but not those of MMP2 at 24 hours (FIG. 9A). Coincidingly, migration of SF295 cells was robustly inhibited by the 24 h-treatment of TGX-221 (FIGS. 8A-8D). These results indicate that RAC1/CDC42/MMP9 acts downstream of p110β to regulate GBM cell migration/invasion (FIG. 9C).

Upon inhibition of PI3K/AKT, the transcription factor FOXO3 stimulates apoptosis in GBM cells by increasing the expression of BIM. Hence, it is possible that the GBM selective survival factor p110β inactivates FOXO3/BIM to suppress apoptosis. By treating SF295 cells with 25 μM TGX-221 for 96 hours, a setting that activates apoptosis in Applicant's previous work, levels of pFOXO3T32 (phosphorylated and inactivated by AKT) was significantly decreased, coinciding with a remarkable increase of BIM-EL, BIM-L, BIM-S (apoptotic forms of BIM) as well as cleaved caspase 3 (c-CASP3, an apoptosis marker; FIG. 9B). Hence, p110β promotes GBM cell survival via repressing the apoptosis inducers FOXO3 and BIM (FIG. 9C).

A unique 18-amino-acid motif in p110β

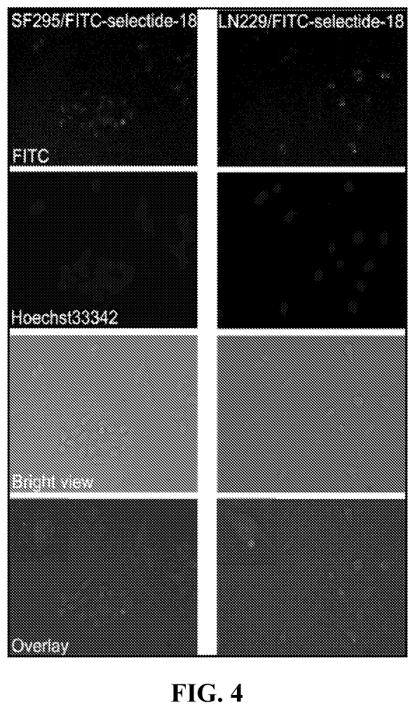

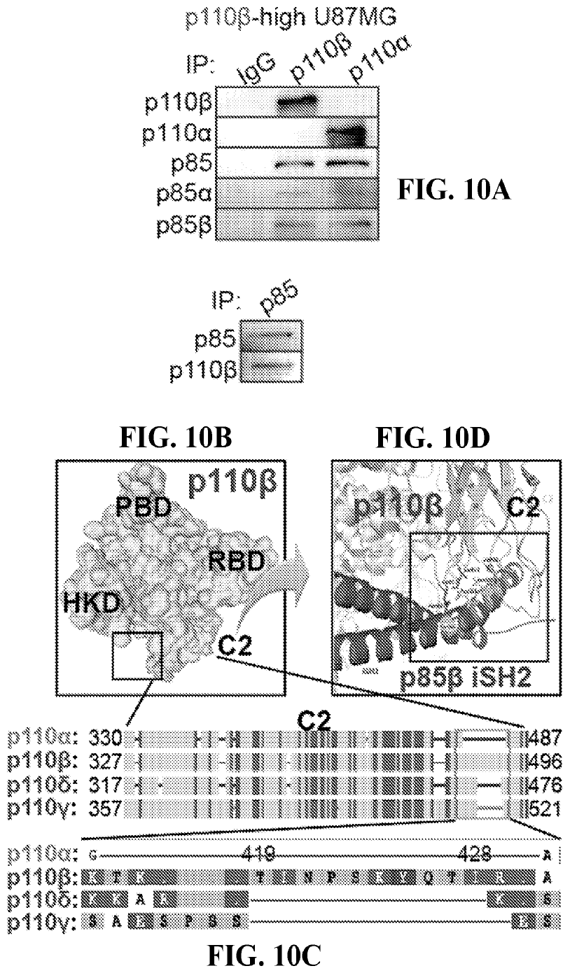

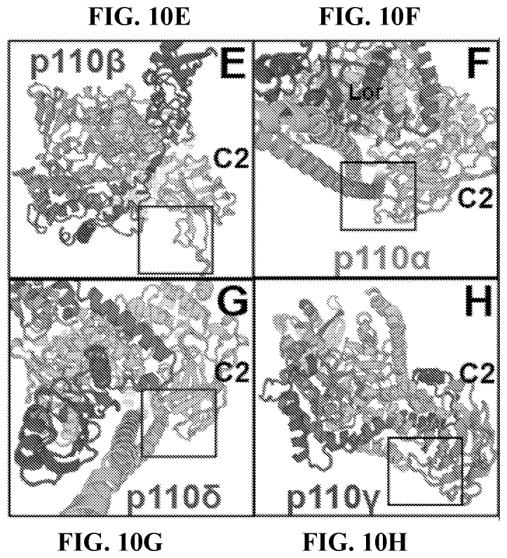

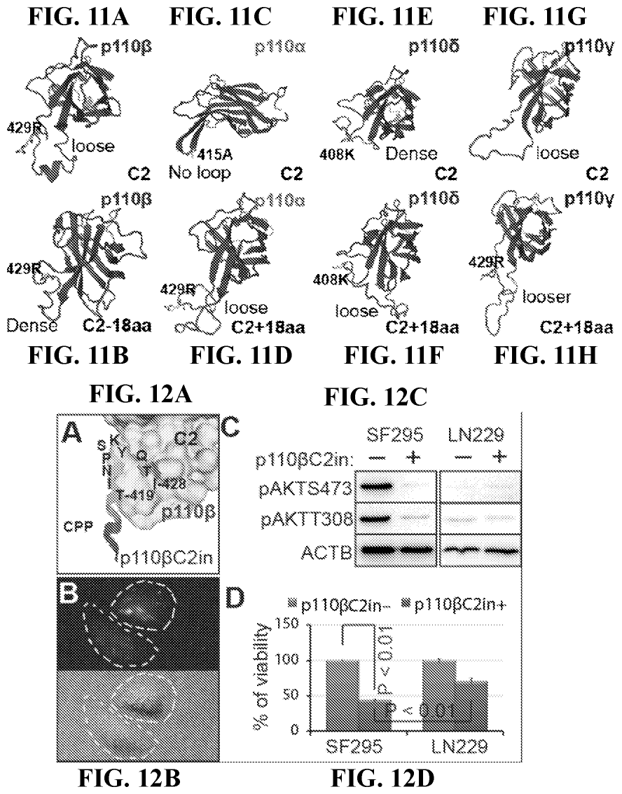

p110 activity is tightly controlled by regulatory subunits. One regulatory subunit p85 binds one p110 to form a complex. For example, p110α/p85 and p110β/p85 complexes were detected in p110βhigh U87MG cells (FIG. 10A). p110 proteins consist of four domains (FIG. 10B): p85-binding domain (PHD), Ras-binding domain (RBD), C2 domain, and helical/kinase domain (HKD). Alignment of amino acid sequences using the NIH constraint-based multiple alignment (COBALT) found that only C2 domains showed a discrete consensus of amino acid sequences amongst four p110 isoforms (FIG. 10C, top panel; indicated by grey bars and red lines). Other p110 domains such as PHD, RBD, and HKD were highly homologous. Noteworthy, TKKSTKTINPSKYQTIRK (SEQ ID NO: 3) in p110β was not found in other p110s (FIG. 10C, bottom panel). This motif locates on the surface of the C2 domain which forms a wide groove together with the HKD, where the p85β iSH2 domain is inserted (FIGS. 10B, 10D, and 10E; boxes). In contrast, no or a narrow groove was found in p110α, p110δ, or p110γ (FIG. 10F-10H). To test whether the TKKSTKTINPSKYQTIRK (SEQ ID NO: 3) motif is important for maintaining 3D conformations of p110β, Applicant remodeled C2 domains using the SWISS-MODEL program. Deleting the 18-amino-acid motif in p110β converted the loose C2 domain (FIG. 11A) into a condensed one (FIG. 11B), whereas insertion of this motif into p110α (FIG. 11C; no loop), p110δ (FIG. 11E; dense), or p110γ (FIG. 11G, loose) resulted in a loose or a looser conformation (FIGS. 5D, 5F, and 5H). These results suggest that this 18-amino-acid motif shapes p110β C2 domains. Applicant next synthesized a peptide drug by fusing TKKSTKTINPSKYQTIRK (SEQ ID NO: 3) with a cell-penetrating peptide (CPP) RRRRRRRR (SEQ ID NO: 4) to yield a p110β selective inhibitory peptide dubbed p110βC2in (FIG. 12A). This peptide was found inside of cells (FIG. 12B). p110βC2in, but not the scramble peptide (control), blocked AKT activity (FIG. 12B) and suppressed cell viability (FIG. 12C) in p110βhigh SF295 cells. Hence, this 18-amino-acid motif is important for p110β activation. FIG. 12D shows results from an MTS Cell viability assay.

Only p110β Controls GBM's Response to Temozolomide (TMZ)

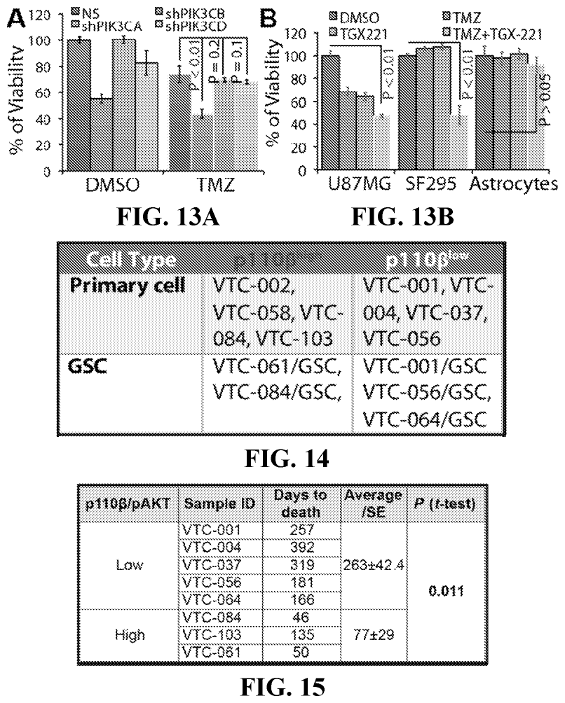

In dividing cells, O6-methylguanine lesions activated by TMZ induce double-stranded DNA breaks and eventually GBM cell death. However, substantial resistance develops to TMZ in GBM patients and significantly decreases clinical efficacy. The PI3K signaling pathway plays a critical role in TMZ's responses to GBM. However, whether PI3K isoforms exhibit different activities to TMZ sensitivity remains elusive. Applicant found that knockdown of PIK3CB not only decreased cell viability, but also induced a further inhibition of cell viability in conjunction with TMZ, whereas depletion of PIK3CA or PIK3CD failed to sensitize GBM cells to TMZ (FIG. 13A). Congruently, TGX-221 also increased TMZ sensitivity in p110 high U87MG and SF295 cells and, more importantly, this combination did not harm astrocytes (FIG. 13B). Hence, selectively targeting p110β is an effective approach for developing combinational therapies for GBM.

Harnessing the Unique Motif in p110β is Innovative to Develop a New Class of p110β-Selective Inhibitors

Tumor progression/recurrence in GBM is inevitable, mostly because GBM cells and GSCs are refractory to current therapies. pan-PI3K inhibitors are non-selective and often yield significant side effects. Applicant finds that PIK3CB defines GBM patients with high risk of tumor recurrence and that p110β is pivotal to the invasion/migration and survival of GBM cells and GSCs. Selectively targeting p110β therefore represents an innovative and effective therapeutic approach. However, p110β-selective inhibitors on the market only modestly slows down tumor growth in mice, suggesting that targeting p110β is not an easy task. Based upon the above results, p110βC2in comprising an 18-amino-acid motif exclusively found in p110β, is a new p110β-selective inhibitor. Harnessing this unique motif in p110β is therefore an innovative approach for therapeutic intervention. It is also imperative to investigate the therapeutic potential of p110βC2in in conjunction with the chemo drug TMZ or immunotherapies such as immune checkpoint inhibitors. This is further explored in this Example.

### Objective Measurement of Peptide Activity and Efficacy

(1) STORM super-resolution microscopy to study protein-protein interactions at nanoscale; (2) 3D invasion assay using Matrigel, astrocyte scaffold, and ex vivo cultured brain slices to determine cell migration/invasion; (3) In vivo GBM models to monitor migration/invasion of fluorescent cells in the mouse brain; (4) Combinational use of GBM mouse models: a genetically modified mouse model testing murine individual PI3K isoforms, a patient-derived xenograft (PDX) model to test human GBM cells and GSCs, and immunocompetent mouse models to monitor immune responses in mice treated with PI3K inhibitors and immune checkpoint inhibitors. Without being bound by theory, it is believed that p110β controls GBM progression and, as such, is an actionable drug target for GBM. Objective measures such as those described herein can be used to evaluate GBM response to these peptides and combination therapies described herein. Such methods can be extrapolated and adapted by those of ordinary kill in the art to evaluate determine the efficacy on other cancers and diseases.

Determination of how the 18-Amino-Acid Motif TKKSTKTINPSKYQTIRK (SEQ ID NO: 3) Exclusively Found in p110 Beta Controls p110β Activation

Applicant has shown that p110 C2 domains display considerably diverse conformations (FIGS. 10A-10H and 11A-11H). Deletion of TKKSTKTINPSKYQTIRK (SEQ ID NO: 3), a unique motif found in p110β C2 domain, transforms a loose C2 into a dense conformation. Contrariwise, insertion of this motif into C2 domains of non-p110β isoforms leads a loose C2 domain. The importance of this motif is further supported by the finding that a mimetic peptide p110βC2in, composed of RRRRRRRR (SEQ ID NO: 4) and TKKSTKTINPSKYQTIRK (SEQ ID NO: 3), inactivated AKT and decreased cell viability (FIG. 12A-12D). Given that TKKSTKTINPSKYQTIRK (SEQ ID NO: 3) locates on the surface of the C2 domain facing the groove between C2 and HKD where the p85 iSH2 domain binds (FIG. 10A-10H), that p85 forms a complex with p110, and that p110 activates RAC1/CDC42/MMP9 to induce cell invasion and inactivates FOXO3A/BIM to suppress apoptosis (FIGS. 8A-8D and 9A-9C). Without being bound by theory, it is believed that: (1) The TKKSTKTINPSKYQTIRK (SEQ ID NO: 3) motif stabilizes p85/p110β complexes and selectively activates p110β; (2) p110βC2in destabilizes p85/p110β, thus inactivating p110β; and (3) p110βC2in inactivates RAC1/CDC42/MMP9 and activates FOXO3A/BIM to regulate cell invasion/survival.

### Materials and Methods

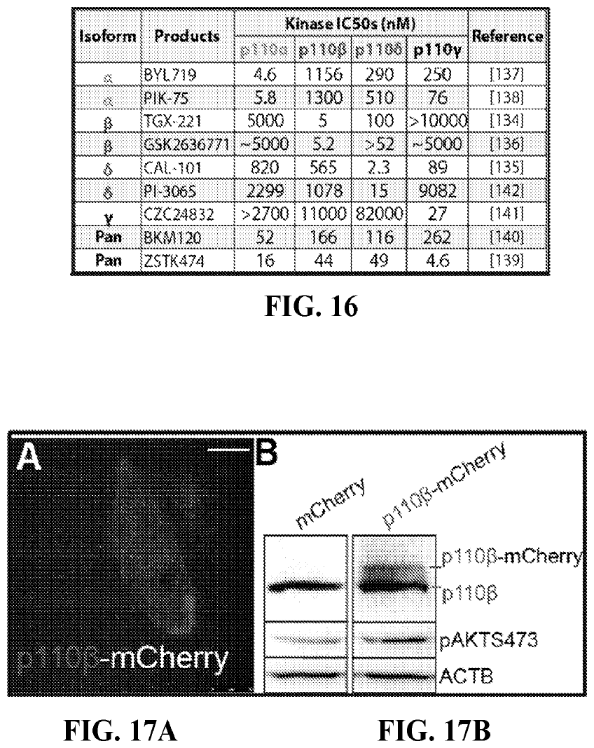

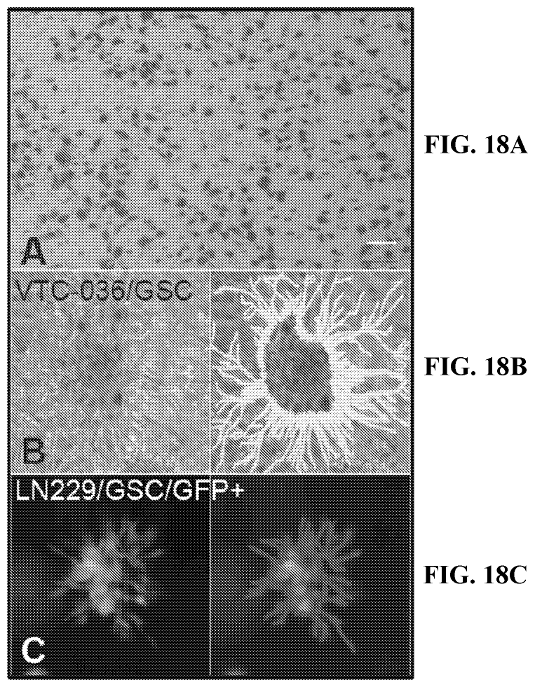

GBM cells: A panel of primary GBM cells and GSCs derived from specimens of patients with newly diagnosed GBM can be employed. Applicant has collected more than 50 GBM specimens, from which primary GBM xenolines and GSC lines are prepared (FIG. 7A-7C and). GBM samples can be further collected to increase the size of our current pool of primary GBM lines and GSC lines. Based on immunoblotting analysis of Applicant's existing pool, Applicant chose several lines of p110β^{high} and p110β^{low} primary GBM cells or GSCs (FIG. 14). Notably, p110β^{high} lines such as VTC-084, VTC-084/GSC, VTC-103, and VTC-061/GSC were from patients with a much shorter life span (FIG. 15), demonstrating strong clinical relevance of the data herein. Some primary/GSC lines were from the same patient (e.g., VTC-084 and VTC-001), which can provide additional insights into how PI3K isoforms differentially regulate differentiated GBM cells and GSCs from the same patient specimen. Astrocytes and neural stem cells (NSCs) can be used as the control.

GBMs harbor 10 to 30% of mutations in genes involved in PI3K signaling such as PTEN (phosphatase and tensin homolog, an inhibitor of PI3K pathway), PIK3CA, and PIK3R1. Hence, one possible caveat of GBM lines used herein is the lack of genetic information pertaining to PIK3CA active mutations. While Applicant has found that PIK3CA mutations are dispensable in GBM patient progression, certain mutations do result in p110-independent activation of AKT. This can be addressed by screening for gain of function mutations in PIK3CA such as H1047R, E545K, and others in Applicant's expanding pool of GBM lines using PCR-amplification followed by regular DNA sequencing. GBM primary lines with active PIK3CA mutations can be excluded. Recently, molecular subtypes of GBM (classical, proneural, and mesenchymal) were revealed based upon differences in gene expression profiles. However, once IDH1 mutation status is adjusted, there is not much of a difference in patient survival among these subtypes in GBM. In addition, this molecular heterogeneity even occurs in the same patient. It is therefore difficult to define GBM cells and GSCs among subtypes. Nonetheless, Applicant's expanding pool of GBM cells can include different GBM subtypes that can inform Applicant of the difference in GBM progression among them. Results can be verified by NSCs transformed with oncogenes and tumor suppressor genes, which mimic GBM subtypes (see below).

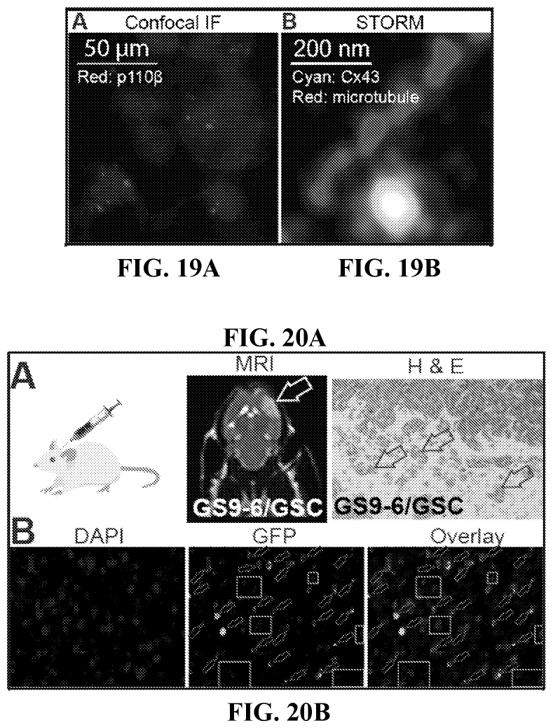

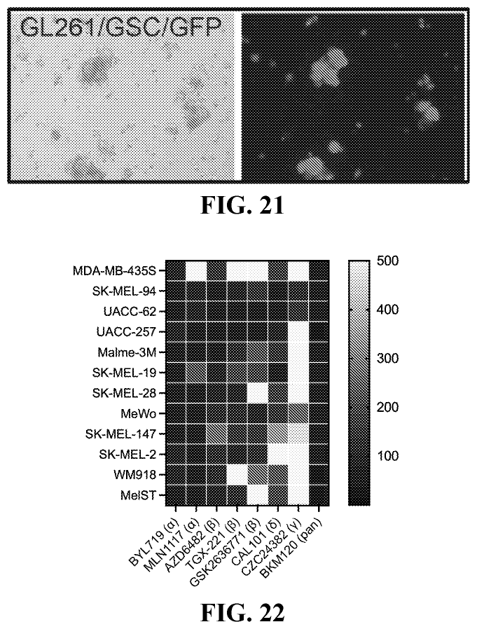

Reagents. To maintain the rigor and reproducibility of our research, multiple different approaches can be used as described below. (1) shRNA knockdown. Two different shRNAs can be used to knock down individual p110 isoforms. Knockdown can be assessed by immunoblotting. A non-silencing (NS) shRNA can be the control. (2) CRISPR-Cas9 knockout. Two different gRNAs can be used to ablate individual PI3K isoforms. An NT gRNA can be the control. Applicant has successfully knocked out p110 genes using CRISPR-Cas9 (FIG. 8A-8D). Gene ablation can be validated using RT-PCR and immunoblotting. (3) Chemical compounds. Different PI3K inhibitors can be used (FIG. 16). Dimethyl sulfoxide (DMSO) can be used as a control. Reagents can, where available, be purchased from a commercial source such as Sigma, Addgene, Selleckchem, or Abcam. (4) Ectopic expression. To objectively characterize and validate the p110β 18-amino-acid motif (SEQ ID NO: 3), overexpression wild-type or active mutants of p110s can be used: pCMV-p110-mCherry encoding a wide-type p110; pCMV-p110βC2-18aa-mCherry encoding a mutant p110β with no TKKSTKTINPSKYQTIRK (SEQ ID NO: 3); pCMV-p110αC2+18aa-mCherry encoding a mutant p110α having an 18-amino-acid motif, pCMV-p110δC2+10aa-mCherry encoding p110δ harboring the missing 10aa (FIG. 11C); pCMV-p110γC2+12aa-mCherry encoding p110γ harboring the missing 12aa (FIG. 11C). Mutants can be generated by site-directed mutagenesis. The PI lab has extensive experience in this technique. The empty vector is the control. Applicant has verified the expression of p110β-mCherry using pCMV-p110β-mCherry (FIG. 17A-17B).

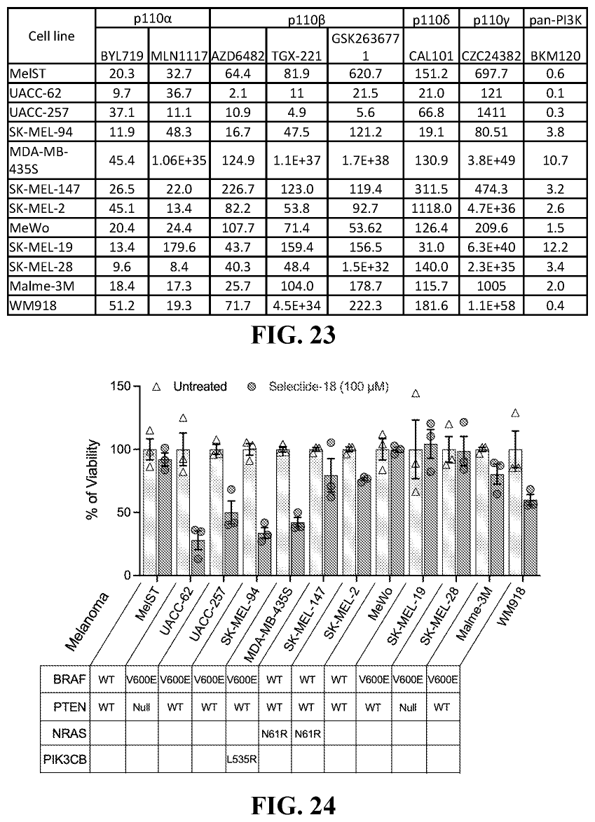

Assays. Multiple assays can be used to monitor cell migration and invasion. A wound-healing (cell migration) assay (FIG. 9A) and/or 3D invasion assay can be done in three model systems: (1) reconstituted basement membrane using Matrigel with reduced growth factors to test GSCs (FIG. 18A), (2) astrocyte scaffold to mimic the brain interstitium, and (3) ex vivo cultured mouse brain slices that mimic the normal brain parenchyma. To better monitor cell migration/invasion, GBM cells and GSCs can be labeled with GFP. GFP+ cells sorted by fluorescence-activated cell sorting (FACS) can be cultured in 3D matrix described above and imaged using a light microscope or a Zeiss fluorescent microscope (FIG. 18B-18C). Cell migration distance can be measured using the Axiovision version 4.8 software (Zeiss). Experiments can be repeated, and replicates can be used to obtain statistical power, the numbers of which will be appreciated by those of ordinary skill in the art.

This 3D invasion assay determines cell motility spatially and temporally and has been used previously in GBM studies. Cell survival and death can be measured using assays established by Applicant or commercially available kits. These assays are: (1) MTS assay (Promega) to determine cell viability; (2) Trypan blue staining to count live cells; (3) Caspase 3/7 activity assay (Promega), immunoblotting of c-CASP3, or annexin V staining (Sigma) followed by FACS to detect apoptosis; (4) LDH-Glo™ cytotoxicity assay (Promega) to measure necrosis; (5) Propidium iodide staining (Sigma) followed by FACS to determine cell cycle arrest; (6) P-galactosidase staining (Cell Signaling Technology) to detect senescence; (7) Colony formation assay to determine anchorage independent growth. Apoptosis assays can be verified by Z-VAD-FMK, an apoptosis inhibitor.

Similar to their normal counterpart NSCs, GSCs can self-renew (copy themselves) and differentiate (converting into other types of cells). Hence, the self-renewal of GSCs can be evaluated using the serial dilution assay to assess the capability of serially diluted GSCs (1 to 100 cells per well) to form spheres. The differentiation of GSCs can be determined by immunoblotting or IF staining of markers for stem or non-stem cells. Markers for GSCs include nestin, prominin-1 (CD133), CD15/SSEA1, A2B5, and LICAM. Non-stem cell markers are 111 tubulin (immature neuron), myelin basic protein (MBP; oligodendrocyte), SOX10 (oligodendrocyte), glia fibrillary acidic protein (GFAP; astrocytes), and ALDH1L1 (astrocytes). Antibodies are available from Abcam and Cell Signaling Technology.

### Method Design

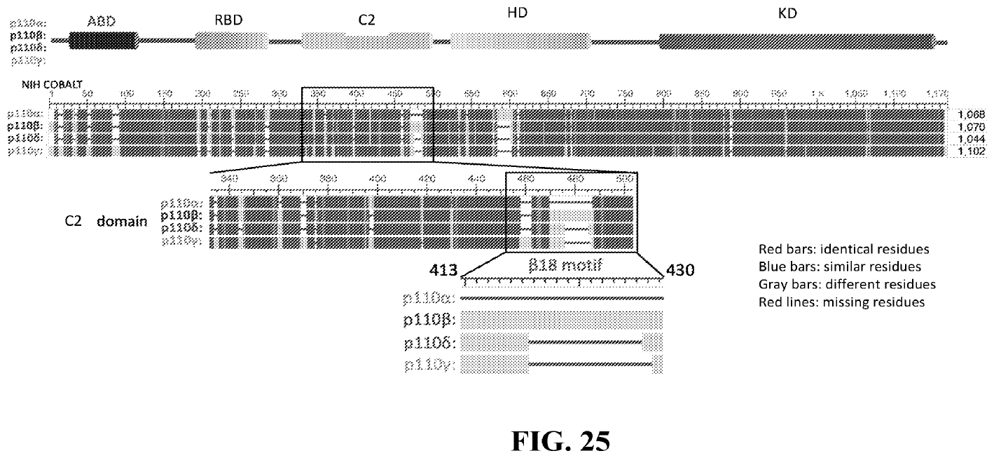

Individual residues in TKKSTKTINPSKYQTIRK (SEQ ID NO: 3) can be evaluated for their ability to regulate the stability and function of p110β/p85 complexes. Three different objective analyses can be used for objectively determining residues involved in regulating the stability and function of p110p/p85 complexes: (1) To determine the role of the 18-amino-acid motif in p110β/p85 complex, p110β^{high} GBM cells and GSCs can be treated with NT gRNA or a p110β gRNA to generate p110j-deficient cells. Cells can then be transfected with pCMV-mCherry, pCMV-p110β-mCherry, or pCMV-p110βC2-18aa-mCherry. (2) To determine the role the 18-amino-acid motif in p110α/p85, p110δ/p85, or p110γ/p85 complexes, p110β-deficient cells can be transfected with pCMV-mCherry, pCMV-p110αC2+18aa-mCherry, pCMV-p110δC2+10aa-mCherry, or pCMV-p110γC2+12aa-mCherry. (3) To determine which residues are important to p110β C2 domain, individual residues in TKKSTKTINPSKYQTIRK (SEQ ID NO: 3) can be deleted in pCMV-p110β-mCherry. Resulting plasmids can be transfected into p110β-deficient cells. Expression of wild-type and mutant proteins can be verified using immunoblotting. The binding between p85 and wild-type or mutant p110 proteins can be monitored using co-IP and validated using mass spectrometry. Endogenous p85 (visualized by a GFP-conjugated p85 antibody) can also be co-localized with wild-type or mutant p110 proteins using confocal microscopy or STORM super-resolution microscopy (FIG. 19A-19B). Cell membrane can be stained with the far-red fluorophore MemBrite™ Fix 680/700 (Biotium) to detect membrane-bound (perhaps active) p85 (green)/p110β (red) complexes. Activity of PI3K can be monitored either using an enzyme-linked immunosorbent (ELISA) assay to measure the ratios of PIP3 and PIP2 using specific antibodies against these lipids or by immunoblotting pAKTs and pGSK3βS9. Activity of RAC1/CDC42 or FOXO3 and levels of MMP9 or BIM isoforms can be determined by detecting pRAC1/CDC42S71, pFOXO3T32, MMP9, or BIM, respectively. Cell migration/invasion, cell survival/death, and GSCs' self-renewal/differentiation can be assessed using the aforementioned approaches.

Without being bound by theory, it is believed that deletion of TKKSTKTINPSKYQTIRK (SEQ ID NO: 3) in p110β C2 domain can destabilize p110β/p85, inactivate PI3K/AKT, block cell invasion/survival, inhibit GSC's self-renewal, and induce GSC's differentiation; (2) Insertion of this 18-amino-acid motif can enable non-p110β proteins to fully or partially compensate the loss of endogenous p110β; (3) Deletion of lysine or arginine residues at both ends of 18-amino-acid motif can disassemble p110p/p85 complex and block p110β function, given that acidic residues in p85 iSH2 domain contact p110β C2 domain; and (4) Deletion of non-charged residues will not affect p110β 's function.

The cellular uptake and localization of p110p3C2in can be evaluated. To determine cellular uptake and half-life of peptides, p110βC2in and the control C2Scramble peptide (e.g., TYKTSKRISKQTIKKPNT (SEQ ID NO: 3) fused with RRRRRRRR (SEQ ID NO: 4)) can be biotinylated. p110βhigh or p110βlow GBM cells and GSCs as well as astrocytes and NSCs can be treated with biotinylated peptides at various doses (0 to 200 μM) and for different duration times (0 to 10 days). Biotinylated peptides inside cells can be quantified using a colorimetric ELISA kit (Abcam) using an HABA dye-conjugated streptavidin. Peptide half-lives can then be calculated. p110βC2in and C2Scramble can also be labeled with fluorescein isothiocyanate (FITC). FITC-conjugated peptides can be visualized using a confocal microscope at various time points. The PI's lab has detected FITC-p110βC2in in LN229/GSCs (FIG. 12B). Without being bound by theory, it is believed that p110βC2in and C2Scramble can be engulfed by GBM cells rapidly and can be stable in tumor cells for hours or even days, which can help determine half-lives of these peptides.

The inhibitory effect of p110βC2in on p110β activity/function can be evaluated. To objectively determine how p110βC2in works, p110βhigh or p110βlow cells can be treated with 200 μM p110βC2in or C2Scramble as shown in FIG. 12A-12D. Stability of p110β/p85 complexes can be evaluated using co-IP. FITC-conjugated p110βC2in (green) can be used to determine whether p110C2in dissociates p85 labeled with Alexa fluor 345 (blue) from p110β -mcherry (red) complexes in GBM cells and GSCs transfected with pCMV-p110β-mCherry using confocal or STORM microscopy. To determine the effect of p110βC2in on cell viability, GBM cells can be treated with p110βC2in or C2Scramble at various doses and for different duration times. If peptide's half-life is <24-hour, cells can be replenished with fresh peptide daily or every other day. PI3K/AKT activity, cell invasion/survival, and GSCs' self-renewal/differentiation can be monitored as described in Task 1. GBM cells and GSCs can also be treated with p110β inhibitors (FIG. 16). p110β inhibitors block p110β activity by competing for the binding of ATP to p110s, rather than destabilizing the p110β/p85 signaling complexes. Without being bound by theory it is believed that p110βC2in, but not C2Scrambles or PI3K inhibitors, can dissociate p110p/p85 complexes and that p110βC2in and p110β inhibitors can inactivate PI3K/AKT thereby suppressing cell invasion/survival, inhibiting self-renewal of p110βhigh GSCs, and inducing GSCs' differentiation.

These results suggest that the 18-amino-acid motif is important for maintaining p110β 3D conformations and activating AKT. Thus, it is believed that this motif determines stability and activity of p110p/p85 signaling complexes. Synergy of TKKSTKTINPSKYQTIRK (SEQ ID NO: 3) with other motifs the in C2 domain to activate p110β can also be objectively evaluated by any suitable method such as preparing A=a series of deletions in p110β C2 domain using site-directed mutagenesis and measuring activity. (2) Half-life can be modified as desired, such as by using nanomaterials such as poly(lactic-co-glycolic acid) (PLGA) to encapsulate p110βC2in so that peptides will be released slowly inside cells. (3) Potential artifacts of mCherry fusion proteins can be controlled as needed by using unconjugated mutants in pCMV-p110β. Direct physical interactions can be detected using surface plasmon resonance. The activity of TKKSTKTINPSKYQTIRK (SEQ ID NO: 3) on PI3K and tumor cell survival can also be determined by the ectopic expression of this 18-mino-acid motif in GBM cells. Of target effects, such as those generated by a CRISPR/Cas9 system used herein, can be mitigate by using a Cas nickase such as a dCas9 that generates single strand breaks that are repaired by the less error-prone homology-directed repair pathway. Alternatively, inducible systems for shRNA-mediated knockdown (GE Dharmacon) can be used.

Determining that p110β Isoform is the Only or Primary Isoform Important for GBM Invasion and Progression.

GSCs are important for GBM formation and increase the risk of GBM progression and recurrence, because: (1) More than 80% of GBM recurrent tumors localize to the sub-ventricular zone (SVZ) and sub-granular zone where NSCs are enriched; (2) Primary GBM tumors near the SVZ are associated with a higher incidence of distant recurrence; (3) Levels of CD133 (a GSC marker) correlate with the risk of distant GBM recurrence; (4) Drug treatment in primary GBM tumor cells increases the number of GSCs; and (5) Applicant finds that MGMT-deficient GSCs are resistant to TMZ. In addition, invasion of GBM cells and GSCs to surrounding normal brain tissues makes it difficult for neurosurgeons to completely remove all tumor cells, a clinical condition that is thought to be the cause of tumor recurrence. Therefore, determining and monitoring how GBM cells and GSCs invade surrounding normal brain tissue can unveil mechanisms underlying GBM recurrence and provide insight on therapeutic options to halt GBM progression. In line with results in FIGS. 7A-13B, without being bound by theory it is believed that p110β is important for GSCs' tumorigenicity because GSCs induce GBM invasion and progression and that p110βC2in inhibits GSC-initiated tumor formation.

### Materials and Assays

BM mouse models. Two GBM mouse models can be used. (1) Genetically-engineered mouse models (GEMMs) can be used to define in vivo function of p110s, despite that this model is time-consuming and costly. Because ablating Pik3ca or Pik3cb is embryonic lethal, conditional knockout technique can be employed to specifically delete PI3K genes in NSCs. Such NSCs can be transduced with viruses containing a p53 shRNA and one of the following: a cDNA of platelet derived growth factor subunit B (PDGFB), a cDNA of epidermal growth factor receptor (EGFR), or an shRNA of neurofibrotosis type 1 (NF-1). These genetic mutations can transform NSC into tumorigenic stem cells. Transformed NSCs can be injected into the brain of C57BL/6 mice, yielding GEMMs. Because the above oncogenic events occur in different GBM subtypes, these GEMMs can help evaluate GBM subtype-related tumor progression. (2) In the orthotropic (also called patient-derived xenograft, PDX) mouse mode, human GSCs derived from GBM patient specimens can be used. Human GSCs are highly tumorigenic and are the possible cause of GBM recurrence. When GSCs from different GBM subtypes are applied in this model, information on the outcomes of tumor progression among different GBM subtypes is generated. Both mouse GBM models mimic clinical tumor progression/recurrence. Statistics. The tumor size can be analyzed using both parametric and non-parametric approaches (e.g., 2-sample t-test or Welch approximation with equal or unequal variances and Wilcoxon Rank-sum test), when appropriate. For time-to-event data, the Kaplan-Meier approach with the log-rank test can be used. SAS 9.2 can be used for the analysis. 10 mice per group can achieve 80% power to detect an effect size of 1.5 given a significance level of 0.05 using a two-sided, two-sample t-test. Potential confounding risk factors can be evaluated by a multivariable regression modeling approach.

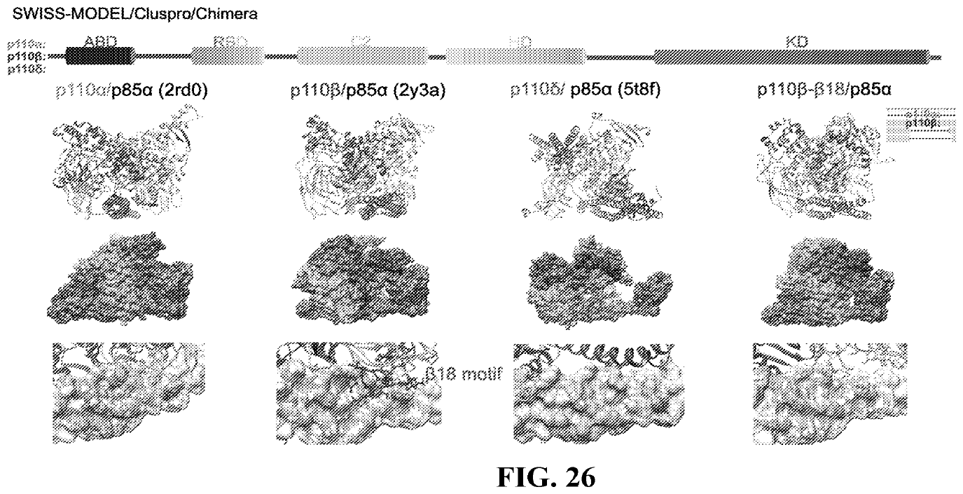

In vivo invasion assay. To measure cell invasion/survival in vivo, two clinically relevant mouse models described above can be used. Transformed NSCs and p110 high GSC lines derived from patient specimens can be labeled by GFP. FACS-sorted GFP+ cells can then be intracranially injected into the striatum of C57BL/6 mice or NOD scid gamma mice. After tumors form in the brain, invasive GFP+ cells can be localized and counted using a confocal microscope and further confirmed by hematoxylin and eosin staining (H&E). Applicant has located infiltrative GS9-6/GSC (FIG. 20A) and U251/GFP+ cells (FIG. 20B) in the brain of NOD scid gamma mice. DAPI (4′,6-diamidino-2-phenylindole) can be used to stain nuclei of mouse and human cells. Because human U87MG cells are able to invade the SVZ where NSCs reside, human GFP+ cells can be quantified in SVZs.

It can be determined whether only p110β is important for GSC to invade the normal brain tissues or grow brain tumors in in vivo GBM models test p110βC2in in GSCs' tumorigenicity in (see below).

Conditional knockout mice can be obtained commercially (e.g., Biocytogen) to prepare GEMMs. In principle, mice possess loxP sites flanking an exon of murine PI3K genes such as p110αtm1Jjz, p110βtm1Jjz, p110δtm2a.1Tnr, or p110γtm1a (EUCOMM) Wtsi C57BL/6 mice (The Jackson Laboratory and GenOway) can be crossed with mice harboring a Cre recombinase driven by the promoter of nestin (NSC marker). The resulting conditional knockout mice can have individual PI3K genes being deleted in NSCs only. To isolate p110α−/−, p110β−/−, p110δ−/−, p110γ−/− NSCs, neuronal cells including NSCs can be isolated from the brain of conditional knockout mice or wild-type C57BL/6 (control) using papain dissociation system (Worthington). NSCs can be further enriched using a CD133 antibody followed by FACS or using the sphere-formation assay. To perform in vivo invasion assay, murine NSCs can be labeled by GFP through GFP plasmid transfection and FACS sorting. GFP+ NSCs can then be transduced with viruses containing combinations of genetic alterations (e.g., p53 loss and amplification of EGFR or PDGFB) described above. 10⁴ transformed NSCs/GFP can be injected into the brain of C57BL/6 mice. PI3K knockout NSCs (four groups) can be compared to PI3K wild-type NSCs (one group). Each group can have 10 mice (Statistical plan) with balanced gender such as equal number of male and female mice. For PDX mouse models, PI3K genes can be knocked out using CRIPSR-Cas9 in two highly infiltrative and tumorigenic p110 high GSC lines (FIG. 14). GSCs can be transfected with a Cas9 plasmid. Cas9-expressing GSCs can be treated by: (1) NT gRNA, (2) p110α gRNA, (3) p110β gRNA, (4) p110δ gRNA, or (5) p110γ gRNA. 104 such cells can be injected into the brain of NOD scid gamma mice. Each group can have 10 mice.

The outcomes of ablations of individual PI3K isoforms can be determined by the mouse survival followed by Kaplan Meier survival analysis. The end point is determined by the severe neurological symptoms associated with brain tumor formation and a significant weight loss. Intracranial tumors can be imaged by magnetic resonance imaging (MRI) using a Siemens 3T Trio whole body scanner with a wrist coil (see Facilities) at the end point. Intracranial tumors can be further analyzed by: (1) Confocal microscopy to determine cell invasion; (2) H&E staining to verify tumor cell invasion; and (3) Immunohistochemical (IHC) analysis or immunoblotting of Ki67 (cell proliferation), cleaved caspase 3 (apoptosis), pAKTs (PI3K activity), pRAC1/CDC42 and MMP9 (invasion signaling), or pFOXO3 and BIM (survival signaling). The Sheng laboratory has the experience in these assays. Without being bound by theory it is believed that (1) Oncogenic mutations (such as p53 loss and EGFR amplification) can transform NSCs with wild-type PI3K genes, which can be indicated by the formation of brain tumors. (2) Ablation of p110β, but not other p110s, can diminish the capability of transformed NSCs or human GSCs to grow a brain tumor in a GEMM or a PDX model, respectively. (3) Only deletion of p110β can inactivate PI3K/AKT. (4) Only deletion of p110β can suppress RAC1/CDC42/MMP9 to block cell invasion, while activating FOXO3A/BIM to induce apoptosis.

To verify the dominant role of p110 in GBM, the efficacy of p110βC2in in suppressing GSC-initiated brain tumor formation can be evaluated. Because few GSCs remain after debulking original tumors in the clinic which cause GBM recurrence/progression, 10,000 transformed mouse NSCs or human GSCs can be used in a GEMM or a PDX model, respectively. GFP+ mouse NSCs with wild-type PI3K genes and human GFP+p110 high GSCs can be intracranially injected into C57BL/6 mice or NOD scid gamma mice, respectively. Mice can then be randomized into six treatment groups: (1) ScrambleC2 (0.75 mg/kg); (2) ScrambleC2 (1.5 mg/kg); (3) ScrambleC2 (3 mg/kg); (4) p110βC2in (0.75 mg/kg); (5) p110βC2in (1.5 mg/kg); and (6) p110βC2in (3 mg/kg). Each group can have 10 mice with balanced gender. Based on experience with peptide drugs, 3 mg/kg is the highest dose of peptide that is tolerable in mice experiments. The treatment of peptide drug can start next day after cell injection. This is to assess whether p110βC2in can prevent the formation of a brain tumor that resembles GBM recurrence. Hence, starting treatments as early as possible is believed to be a better strategy for reducing the risk of tumor recurrence. Peptide drugs can be slowly delivered into the brain using the Alzet brain infusion assembly with osmotic pump. Alternatively, repeated injections of peptide drugs directly through the cell injection site (once a week for maximum three weeks) can be performed. The end point is when mice in the control group develop severe symptoms of tumor-associated neurological dysfunction accompanied with a significant weight loss. Mouse survival, activity of PI3K and its downstream survival signaling and invasion signaling, in vivo cell invasion, and cell proliferation/apoptosis can be analyzed as previously described.

Without being bound by theory it is believed that (1) p110βC2in, but not ScrambleC2, can inhibit the tumorigenicity of transformed mouse NSCs or human GSCs as indicated by the increased mouse survival, decreased tumor volume, attenuation of PI3K and its downstream signaling, reduced levels of tumor proliferation/invasion, and induction of apoptosis. (2) The effect of p110βC2in on tumor formation can be dose-dependent.

Additional methods. To determine if two or more PI3K isoforms are needed for GSCs or transformed NSCs to form a brain tumor, in p110β−/− NSCs or GSCs, shRNAs of other PI3K catalytic isoforms can be used to knock down these isoforms individually. (2) Drugs delivered intracranially still face difficulty in penetrating the blood-brain barrier (BBB). Thus, focused ultrasound can be used to temporarily open the BBB. (3) To provide an alternative approach for in vivo invasion assay, human GBM cells can be immuno-stained by a Cy5 (red)-labeled antibody against human mitochondria (Millopore) and co-localized with GFP. This allows mouse and human cells to be distinguished from one another.

Determine the In Vivo Efficacy of p110βC2in in Conjunction with Temozolomide or Immune Checkpoint Inhibitors

Recurrent and progressive GBMs are resistant to the frontline chemo drug TMZ. Thus, antagonizing TMZ resistance could have a genuine impact on clinical management of recurrent GBMs. Activation of PI3K renders cancer cells resistant to radiation/chemotherapies. Targeting the PI3K pathway as a TMZ-sensitizer is an appealing approach and has been previously explored. However, most of these studies focused on pan-PI3K inhibitors or PI3K/MTOR dual inhibitors. Notably, clinical trials using these combinations show no or modest effects in addition to severe toxicity. Because only knockdown of PIK3CB restored TMZ sensitivity (FIG. 13A-13B), without being bound by theory it is believed that the novel p110β-selective inhibitor p110βC2in sensitizes TMZ-resistant GBM cells to TMZ in vivo.

Recent research has demonstrated a more permissive, dynamic crosstalk between the brain and the peripheral immune system, which challenges the immune privilege model in the central nervous system. This paradigm shift has stimulated research on investigating the possibility of using immunotherapies to treat GBM. Antagonizing immune checkpoint molecules such as cytotoxic T-lymphocyte antigen 4 (CTLA-4) and programmed cell death protein 1 (PD-1) or its ligand (PDL-1) have shown some promising therapeutic benefits in preclinical GBM models, which has encouraged phase I clinical trials using antibodies of CTLA-4 or PD-1 for recurrent GBM patients (NCT02054806 and NCT02311920). Nonetheless, it is anticipated that a highly immunosuppressive microenvironment is formed in GBM, which limits the body's immune response to the tumor. Such an immunosuppressive microenvironment is attributed to a heterogenous cell population in GBM including glioma cells, GSCs, immune cells, and stromal cells. The immunosuppressive role of GSCs has been indicated by the GSC-formed niche which bolsters the secretion of immunosuppressive proteins such as PD-L1 or activates PI3K, STAT3, or hypoxia to inhibit T-cell activation and proliferation, induce T-cell apoptosis, and upregulate immunosuppressive T-regulatory cells. Hence, it is important to break this immunosuppressive microenvironment to increase the sensitivity of GBM to immunotherapies. Because Applicant has shown that PIK3CB/p110 is highly expressed in GSCs and is important for them to grow, without being bound by theory it is believed that p110 inhibitors including p110βC2in booster the in vivo efficacy of immune checkpoint inhibitors.

### Materials and Assays

MZ-resistant GBM mouse model. When tumor-resected GBM patients are treated with TMZ, remaining GBM cells and GSCs undergo a selection of TMZ-resistant clones. To resemble this clinical condition, LN229/GSCs which are relatively less sensitive to TMZ (IC50=100 μM) can be used. 105 GFP+LN229/GSCs can be injected into the brain of 10 Nod scid gamma mice. Mice can be treated with TMZ at escalating doses (from 1 to 5 mg/kg/day through intraperitoneal injection) for 1 to 2 months. Upon detection of intracranial tumors by MRI, tumors can be harvested. Single cell suspension can be made using papain dissociation system. 10 lines of GFP+ TMZ-resistant LN229GSCs can be sorted using FACS and further enriched using the sphere formation assay. TMZ IC50 can be determined and the TMZ-resistant LN229/GSC line with the highest IC₅₀ can be reinjected into the mouse brain to establish a TMZ-resistant GBM model.

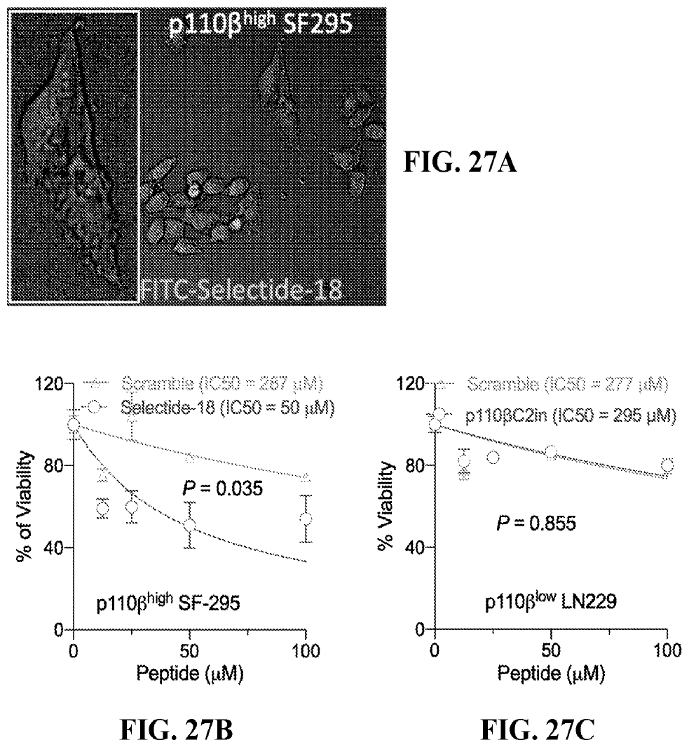

Immunocompetent GBM mouse models: To test immunotherapy in mice, immunocompetent mouse glioma models can be used. GL261 and CT-2A, held by the PI, were derived from mouse malignant glioma resembling grade III glioma (astrocytoma) and grade IV glioma (GBM). GSCs isolated from GL261 or CT-2A and labeled with GFP (FIG. 21) can be injected into the brain of C57BL/6 mice. These immunocompetent mouse models enable us to gauge efficacy of immunotherapies for GBM. In fact, immune checkpoint inhibitors such as anti-PD-1 and anti-CTLA-4 have been tested previously in these immunocompetent mouse models. In the GEMM proposed in Aim 2, murine transformed NSCs can be used in immunocompetent mice. This mouse model can also be used herein to test these immunotherapies.

In vivo efficacy of p110βC2in and TMZ can be evaluated in TMZ-resistant GBM mouse model. 104 GFP+ TMZ-resistant LN229/GSCs, which have been selected by escalated doses of TMZ in mice, can be injected into the brain of NOD scid gamma mice. On next day, mice can receive treatments as reasoned above. Treatment groups can include: (1) TMZ (7.5 mg/kg; intraperitoneal injection/daily); (2) ScrambleC2 (1.5 mg/kg)+DMSO; (3) TMZ+ScrambleC2; (4) p110βC2in (1.5 mg/kg)+DMSO; (5) TMZ+p110βC2in. Applicant intentionally used 7.5 mg/kg TMZ and 1.5 mg/kg p110βC2in based on our previous work on TMZ and a connexin 43 mimetic peptide. Low doses of each treatment can perhaps lower the risk of severe side effects exhibited by combinations. Peptide drugs can be intracranially injected into the brain using the Alzet brain infusion assembly with osmotic pump or repeated intracranial injections as described above. Mouse survival, activity of PI3K and its downstream signaling pathways, invasion of GFP+ cells, and cell proliferation/apoptosis can be analyzed as described above. To monitor the toxicity of the above combinational treatments to normal brain, NOD scid gamma mice without implantation of tumor cells can be used. Toxicity is indicated by neurological deficits such as circling deficit, righting reflex deficit, knuckling, arched back and walking on toes.

Without being bound by theory it is believed that (1) p110βC2in can sensitize TMZ-resistant LN229/GSCs to TMZ, manifested by attenuated PI3K signaling, reduced tumor volume, suppressed tumor invasion, decreased tumor proliferation, and bolster apoptosis in mice treated with p110βC2in and TMZ only. (2) ScrambleC2 will not sensitize LN229/GSC tumors to TMZ. (3) Drug combinations will not induce significant neurological deficits in mice bearing no tumors.

Whether p110βC2in sensitizes GBM tumors to immune checkpoint inhibitors can be determined in immunocompetent mouse models. 10⁴ GFP+GL261/GSCs, GFP+CT-2A/GSCs, or GFP+ transformed NSCs can be injected into the brain of C57BL/6 mice. Next day after cell injection, mice can be randomized into the eight treatment groups: (1) ScrambleC2 (1.5 mg/kg)+IgG; (2) p110βC2in (1.5 mg/kg)+IgG; (3) ScrambleC2+PD-1 antibody (Bioxcell, 100 μg per mouse with repeated intraperitoneal injection every 3 days for 8 maximum treatments); (4) p110βC2 in +PD-1 antibody; (5) ScrambleC2+CTLA-4 antibody (Bioxcell, 100 μg per mouse with repeated intraperitoneal injection every 3 days for 8 maximum treatments); (6) p110βC2in+CTLA-4 antibody; and (7) p110βC2in+PD-1 antibody+CTLA-4 antibody. 100 μg per mouse has been previously used in GL261 or CT-2A mouse models. Each treatment group can have ten mice with balanced gender. Therapeutic responses can be determined by Kaplan Meier survival analysis. Brain tumors can be verified and quantified by MRI. Assays described in above can be used to determine activity of PI3K and its downstream survival and invasion signaling, tumor invasion, tumor proliferation index, and apoptosis. To monitor immune responses, leukocytes can be isolated from tumors and peripheral blood using fluorophore-labeled CD45 antibodies followed by FACS. Tumor-specific and peripheral leukocytes can then be further analyzed by FACS to determine levels of cytotoxic T cells (CD3+CD8+), regulatory T cells (CD3+CD4+FoxP3+), myeloid-derived suppressor cells (CD11b+Gr-1+), dendritic cells (CD11b+CD11c+), microglia (CD11b+F4/80+TMEM119+), M1 macrophages (CD11c-CD11b+F4/80+iNOS+), and M2 macrophages (CD11c-CD11 b+F4/80+CD163+CD200R+). T cells, myeloid-derived suppressor cells, dendritic cells, microglia, and GSCs together constitute an immunosuppressive myeloid cell compartment in the brain tumor. In a different set of experiments, a p110j inhibitor such as TGX-221 (40 mg/kg, intraperitoneal injection) or GSK2636771 (30 mg/kg, intraperitoneal injection) can be used to determine whether these p110β inhibitors can increase sensitivity of immune checkpoint inhibitors.

Without being bound by theory it is believed that (1) p110βC2in, but not ScrambleC2, can significantly inhibit the formation of brain tumors and improve mouse survival, in combination with antibodies against PD-1 and/or CTLA-4; (2) Combination of p110βC2in and PD-1 or CTLA-4 antibody can significantly increase levels of CD8+ cytotoxic T cells and decrease levels of CD4+ regulatory T cells, microglia cells, myeloid-derived suppressor cells, dendritic cells, and macrophages in the tumor and peripheral blood. (3) Combination of TGX-221 or GSK2636771 with immune checkpoint inhibitors can substantially block the growth of tumors in the mouse brain.

Other Approaches (1) the combinational use of p110βC2in, TMZ, and an immune checkpoint inhibitor is effective as a trimodal treatment for GBM can be tested. This hypothesis is supported by the recent finding that combinations of TMZ and immune checkpoint inhibitors are more effective than each monotherapy. (2) lower doses of each drug in the combination can be employed to reduce any observed toxicity. (3) To provide an alternative mouse model that monitors the effect of immunotherapies in activating human immune cells, humanized immune checkpoint mice (Biocytogen) can be used, such as B-hPDL1, B-hPD-1/hPDL-1, B-hCTLA4 C57BL/6 mice harboring splenocytes expressing human checkpoint antigens. (4) Drug combinations can be tested in the GEMM described in detailed above.

Selectide-18 is a p110β-Selective Inhibitor Specifically for p110β^{high} Glioblastoma

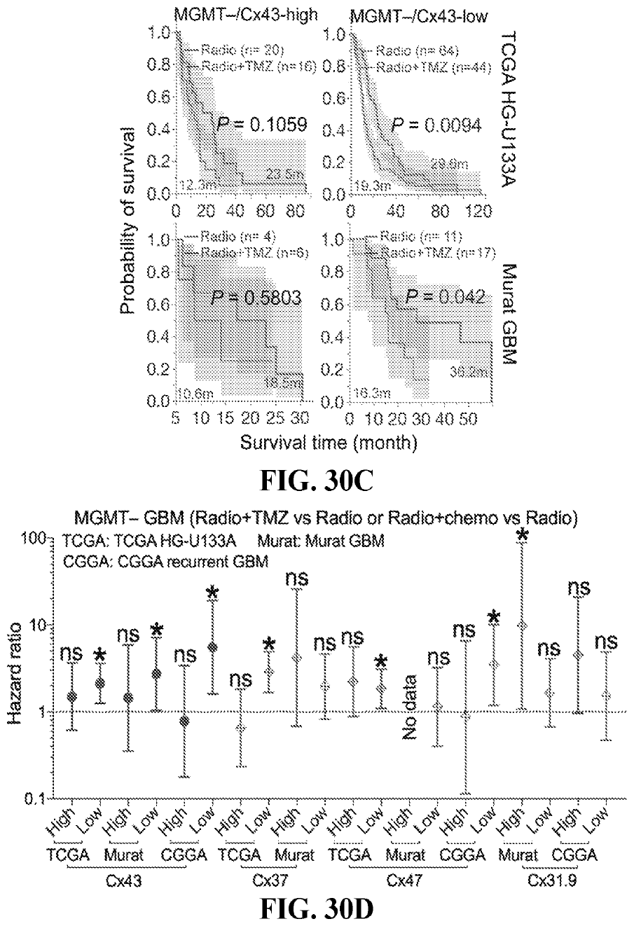

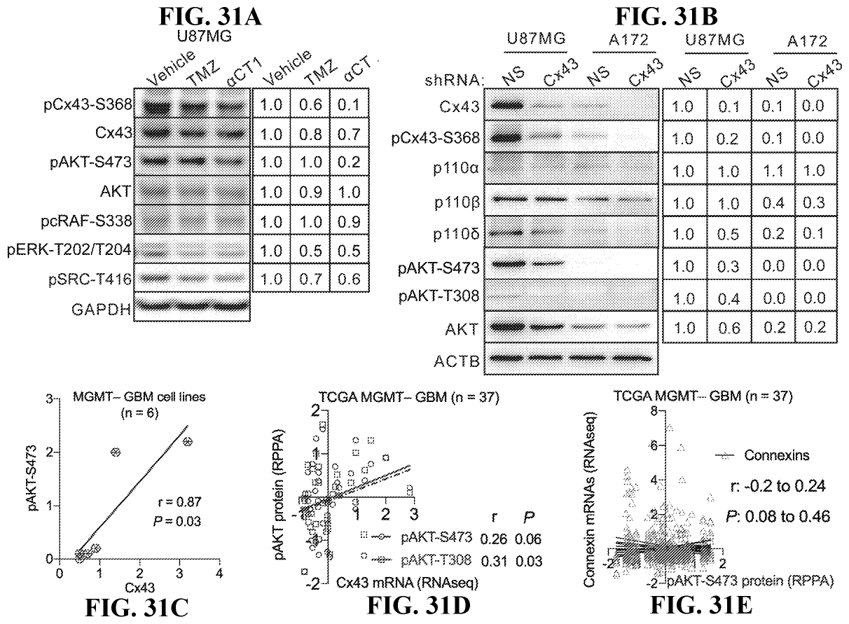

FIG. 27A-27G demonstrates that selectide-18 is a p110beta-selective inhibitor and is effective in e.g., p110beta^{high} glioblastoma. FIG. 28 further demonstrates that that selectide-18 is a p110beta-selective inhibitor and is effective in e.g., p110beta^{high} glioblastoma.

### PIK3CB/p110b Targeting Peptides

PI3K signaling is important for glioblastoma cells to survive. Recent observations indicate that a specific PI3K catalytic isoform PIK3CB/p110b (p110Beta) dominates in the activation of PI3K signaling and is more important than other subunits in promoting cell survival and proliferation. Hence, selectively targeting PIK3CB/p110b is a viable therapeutic approach for this deadly brain cancer and other cancers and diseases with aberrant PI3K signaling driving at least part of the pathology or symptoms. It has been shown that current chemical compounds selectively targeting PIK3CB/p110Beta actually exhibit low selectivity on this subunit and limited therapeutic efficacy. To develop an innovative approach to targeting PIK3CB/p110Beta, Applicant analyzed the protein sequences in all four PI3K catalytic subunits and identified a unique protein sequence in PIK3CB/p110Beta that is different from the other three isoforms. As shown in the section of this Appendix titled, “PIK3CB_p110Beta Targeting Peptides as a GBM Therapy”, a strategy for studying PIK3CB/p110Beta in GBM, this peptide (TKKSTKTINPSKYQTIRK (SEQ ID NO: 3)) fused with a cell-penetrating peptide RRRRRRRR (SEQ ID NO: 4) (termed p110bC2in) blocked PI3K signaling and induced significant growth inhibition in GBM. To further characterize this approach and identify a best strategy of utilizing this peptide as a cancer therapy, Applicant tested two different versions of this peptide: (1) Ante-Selectide-18 (RQPKIWFPNRRKPWKKTKKSTKTINPSKYQTIRK (SEQ ID NO: 2)), in which this 18 amino acid p110b peptide was fused with an antennapedia cell penetrating sequence (RQPKIWFPNRRKPWKK (SEQ ID NO: 6)), and (2) Selectide-18 (TKKSTKTINPSKYQTIRK) (SEQ ID NO: 3), which only contains this p110b targeting sequence. See also Table 1 below.

TABLE 1

| Name | Sequence | SEQ ID NO: |
|---|---|---|
| p110bC2in | RRRRRRRRTKKSTKTINPSKYQTIRK | 1 |
| (p110βC2in) | | |
| Ante-Selectide-18 | RQPKIWFPNRRKPWKKTKKSTKTINPSKYQTIRK | 2 |
| Selectide-18 | TKKSTKTINPSKYQTIRK | 3 |

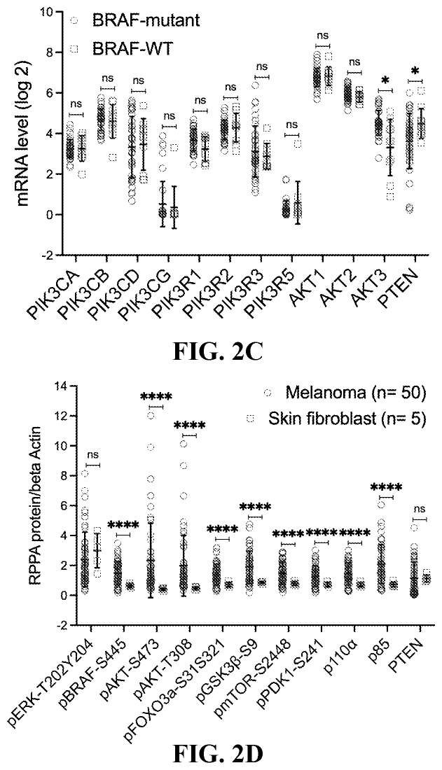

In FIG. 3 Applicant tested whether Ante-Selectide-18 affects PI3K signaling in GBM cells. In SF295 cells treated with 200 μM Ante-Selectide-18, levels phosphorylated form of AKT (a substrate of PI3K) was substantially reduced, coinciding with a remarkable decrease of active forms of GSK3b and RAC1/CDC42, two downstream targets of PI3K/AKT. In contrast, another PI3K/AKT downstream target MTOR was not inhibited by this peptide. Therefore, Ante-Selectide-18 selectively blocks PI3K/AKT/GSK3b and PI3K/AKT/RAC1/CDC42 signaling without affecting MTOR. Our results demonstrate that Ante-Selectide-18 is an alternative form of p110bC2in, which may be a p110b-selective inhibitor.

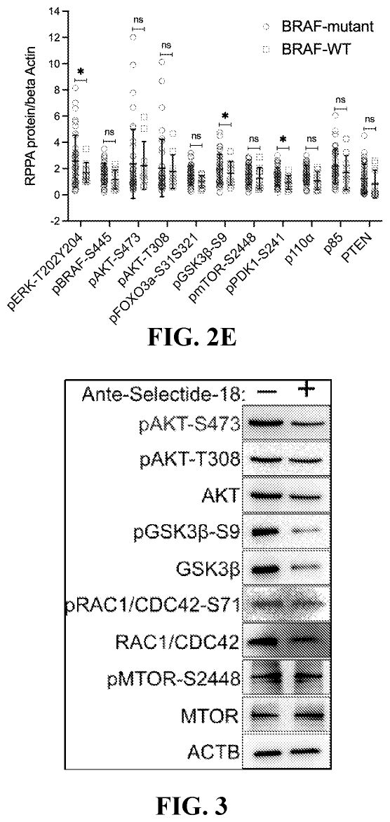

In FIG. 4, Applicant tested Selectide-18. Because Selectide-18 was not fused with any cell penetrating peptides, Applicant added a fluorophore, FITC, to its N-terminus and monitored the cellular uptake. After 1-hour exposure to FITC-Selectide-18, cells were imaged live using an inverted fluorescence microscope. Applicant found that FITC-Selectide-18 was taken up by both SF295 and LN229 cells efficiently. The inset figures shown in the panel Overlay showed that green dots (FITC-Selectide-18) were found in the cytosol. Hence, without a cell-penetrating peptide, Selectide-18 itself can be taken up by GBM cells, suggesting that Selectide-18 can be a p110b-selective inhibitor, similar to p110bC2in and Ante-Selectide-18.

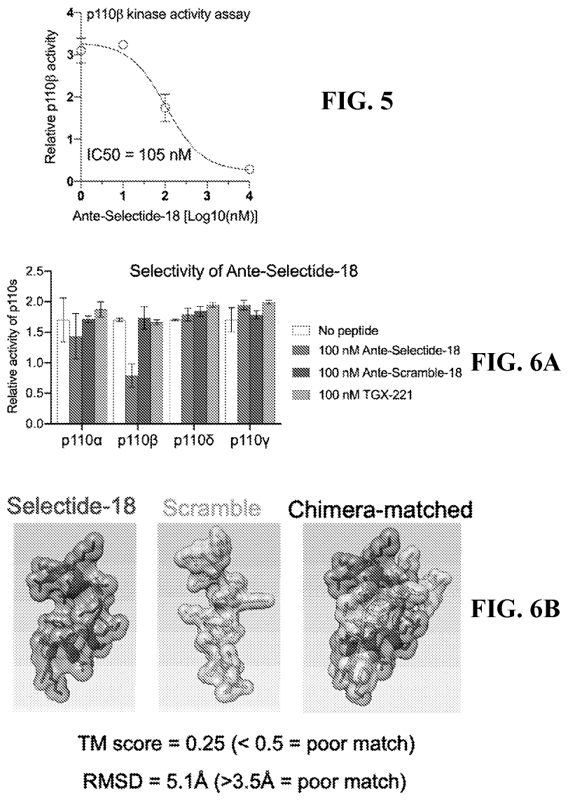

In FIG. 5, Applicant shows the activity of the p110b catalytic subunit, measured via Enzyme-Linked Immunosorbent Assay (ELISA), is inhibited by Ante-Selectide-18 peptide and has a Half Maximal Inhibitory Concentration (IC50) of 105 nM (Nanomolar). To further Applicant's results, in FIG. 6A-6C Applicant models and shows, via ELISA assay, the Ante-Selectide-18 peptide only targets to and inhibits the activity of p110b and does not affect the other PI3K subunits p110c, p110δ, and p110γ at a concentration of 100 nM. Applicant also included a control scramble peptide containing the antennapedia sequence followed by the amino acid sequence of Ante-Selectide-18 randomly scrambled. As a further control Applicant included the p110b isoform inhibitor TGX-221. Applicant reports that this drug is not effective at inhibiting the kinase activity of p110b or other PI3K subunits at 100 nM, showing the promise of the Ante-Selectide-18 peptide as a potent selective p110b inhibitor at a low dose.

### Example 2—Selectides for Melanoma Treatment

### Melanoma

Cutaneous melanoma is a difficult-to-treat cancer. While the 5-year survival rate of localized melanoma is about 99%, this rate drops to 65% or 25% when the tumor becomes regional or distant. Somatic mutations are frequently found in BRAF (50-60%), NRAS (15-30%), NF1 (14%), and PTEN (8˜10%), yielding four molecular subtypes: BRAF, RAS, NF1, and triple wild-type. Amongst all BRAF mutations, BRAFV600E accounts for 75 to 90% of cases. BRAFV600E-specific inhibitors and MEK inhibitors have achieved promising clinical outcomes. However, drug resistance and tumor progression occur inevitably, manifested by the unresponsiveness to these treatments in about 15% of BRAFV600E melanoma as well as de novo or acquired resistance due to the reactivation of MAPK and/or the counteraction by other survival pathways such as phosphoinositide 3-kinase (PI3K). PI3K has four catalytic subunits PIK3CA, PIK3CB, PIK3CD, and PIK3CG (PI3K catalytic subunit α, β, δ, and γ) that encode p110α, β, δ, and γ (also called PI3Kα, β, δ, and γ), respectively. PI3K catalytic subunits form a signaling complex with PI3K regulatory subunits PIK3R1, PIK3R2, or PIK3R3 that encodes p85α, p85β, or p55γ, respectively. Mutations in PI3K genes have been reported in drug-naïve and -resistant melanoma. But the mutation rate is low (<1%). It is well-documented that PI3K activation causes therapy resistance; however, overcoming this resistance through PI3K inhibition has shown disappointing results in the clinic. This is partially because pan-PI3K drugs that block all PI3K subunits are highly toxic.

The role of individual PI3K subunits in melanoma has been contradictory. Deuker et al., found that pan-PI3K inhibitors, but not p110β inhibitors, sensitized mouse BrafV^{600E} melanoma to BRAF inhibitors. In addition, mouse BrafV^{600E}/Pik3caH1047R melanoma depended on p110αH1047R, but not other p110s, to activate PI3K. p110β blockers did not show therapeutic benefits for Rac1P29S melanoma. Moreover, p110α or p110δ inhibitors exhibited strong cytotoxicity to melanoma, whereas p110β or p110γ inhibitors had no or limited effect [44, 56]. These studies suggest that p110β is dispensable for certain melanoma. Contradictory to these studies, the p110β inhibitor SAR260301 together with BRAF/MEK blockers inactivated PI3K and blocked the growth of BRAFV^{600E}/PTENnull human melanomas. Inhibition of p110β, but not other p110s, co-operated with PTEN loss in BRAFV^{600E}/PTENnull melanoma. The concomitant use of the p110β inhibitor AZD8186 and the programmed cell death protein 1 (PD-1) antibody retarded the growth BrafV^{600E}/Ptennull tumors in vivo. These studies have indicated the critical role of p110β in BRAF^{V600E}/PTENnull melanoma. The important role of PI3K in therapy resistance and perplexing results of individual p110s have demonstrated a clinically unmet need for effective PI3K therapies, which has led Applicant to reinvestigate individual p110s in melanoma.

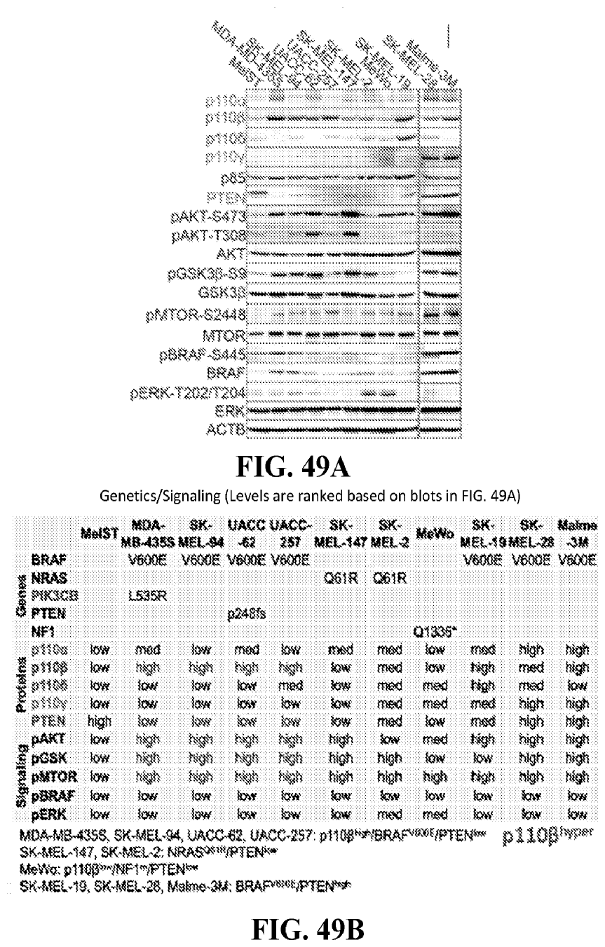

Congruent with Applicant's recent work in brain cancer and results from other studies in breast, colon, and prostate cancer, p110s played different roles in melanoma. p110β were expressed at a much higher level than p110a in BRAFV^{600E}/PTEN^{low} melanoma cell lines (FIG. 48A) and Wistar BRAF^{V600E} patient-derived xenografts (PDXs) with low levels of PTEN (PTEN^{low}, FIG. 48B). p110β levels positively correlated with PI3K activation in BRAFV^{600E}/PTEN^{low} melanoma (FIG. 48C). High levels of PIK3CB mRNA and pAKT-5473 were inversely associated with poor prognosis of BRAF^{V600E} patients (FIG. 48D). p110β is highly expressed in BRAFV^{600E}/PTEN^{low} melanoma and associated with PI3K activation and patient survival.

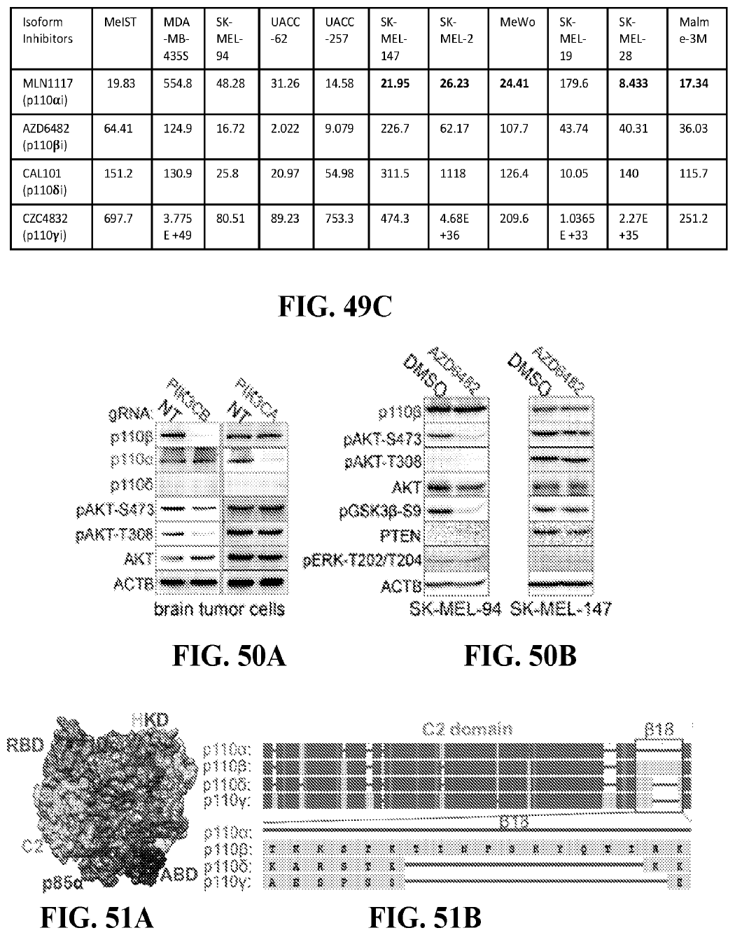

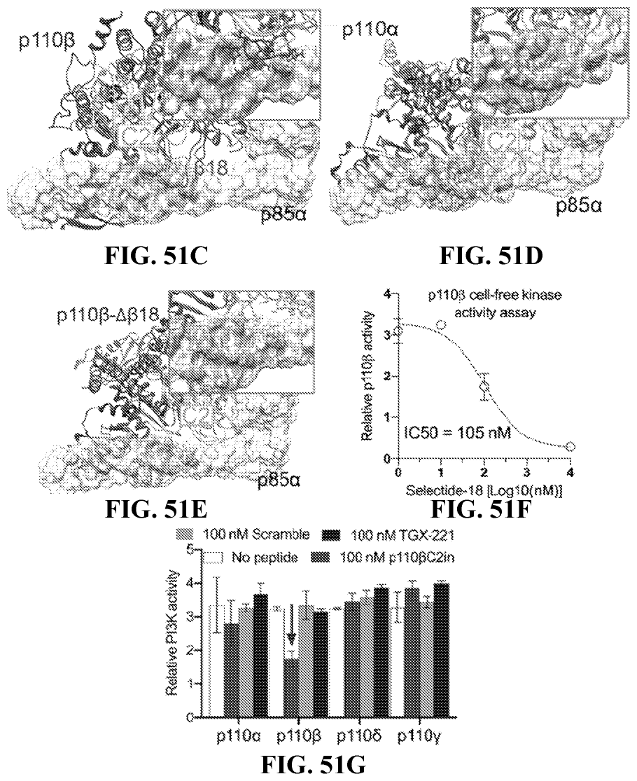

BRAF^{V600E} MDA-MB-435S, SK-MEL-94, UACC-62, and UACC-257 cells had high levels of p110β and low levels of PTEN (p110β^{high}/PTEN^{low}; FIG. 49A-49B). PI3K were hyperactivated in these cells and they are named as p110β^{hyper} hereafter. The p110β inhibitor AZD6482 was cytotoxic to p110β^{hyper} cells, but not melanocyte MelST (FIG. 49C). In contrast, inhibitors of p110α, p110δ, or p110γ (e.g., MLN1117, CAL-101, or CZC24832) did not show a selective inhibition of cell viability. Intriguingly, p110β^{hyper} MDA-MB-435S was not sensitive to AZD6482, possibly due to the PIK3CBL535R mutation in this line (FIG. 49B). p110β^{hyper} brain tumor cells transduced with Cas9 and a guide RNA (gRNA) of PIK3CB depleted p110β and inactivated PI3K, whereas gRNAs of non-targeting (NT) or PIK3CA had no effect (FIG. 50A). Consistently, AZD6482 inactivated PI3K in p110β^{hyper} SK-MEL-94 cells, but not in NRASQ61R/PTEN^{low} SK-MEL-147 cells (FIG. 50B). These results have demonstrated that p110β is critical for the survival of p110β^{hyper} tumor cells.

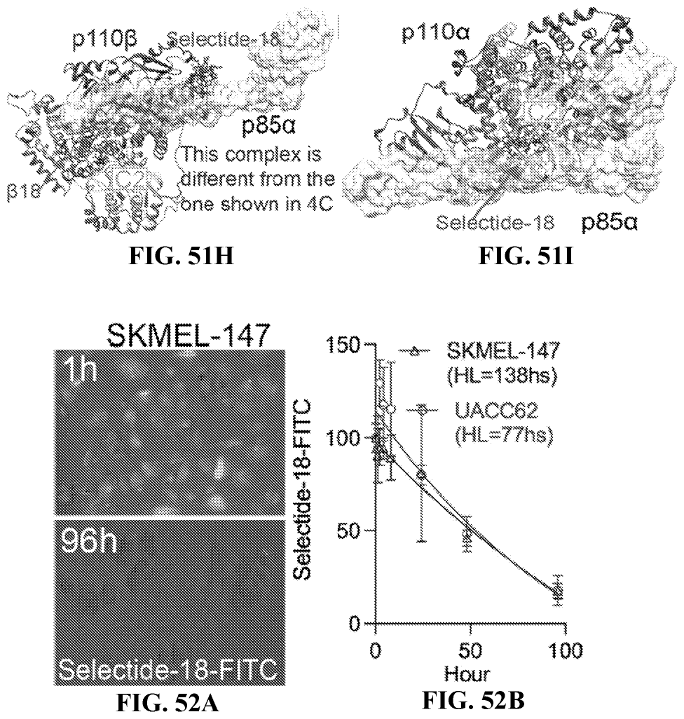

Current p110 isoform inhibitors depend on differential binding activities of these compounds to p110s. While their IC50s are in nano molar range in cell-free assays, p110 isoform inhibitors still exhibit a certain degree of non-selective inhibition to other p110s when applied to tumor cells/tissues. To overcome this challenge, Applicant adopted an innovative strategy to develop p110β drugs. One p110vbinds to one p85 to form a p110/p85vcomplex. p110s consist of four domains (FIG. 51A): p85-binding domain (ABD), Ras-binding domain (RBD), helical/kinase domain (HKD), and C2 domain. Alignment of protein sequences showed a discrete consensus in C2 domains (FIG. 51B, grey bars and red lines). TKKSTKTINPSKYQTIRK (SEQ ID NO: 3) in p110β (termed as 018 and Selectide-18) was not found in other p110s (FIG. 51B). This β18 motif (magenta) locates on the C2 surface (cyan) and forms a groove together with the HKD and ABD, where p85 (dark grey) is inserted (FIG. 51A). In-silico analyses showed that the β18 motif made a close contact with p85α (FIG. 51C, inset figure), yielding a p110β/p85α complex that is different from that of p110α/p85α (FIG. 51D). Deletion of 018 (p110β-Δβ18) significantly changed 3D shapes of p110/p85α (FIG. 51E). Fusing the 018 peptide with a cell-penetrating peptide resulted in a p110p3-mimetic peptide inhibitor termed as Selectide-18 in this Example 2, which inactivated p110β with an IC₅₀ of 105 nM (FIG. 51F). Applicants note the nomenclature change in the peptides from Example 1, where “Selectide-18” was the name applied to the p110beta isoform mimetic peptide (e.g., SEQ ID NO: 3). The reported IC₅₀s of GSK2636771, TGX-221, or AZD6482 are 5, 8.5, or 10 nM, respectively, which are 10- to 20-fold lower than that of Selectide-18. However, 10 nM TGX-221 (equivalent to 100 nM Selectide-18 based on their IC₅₀s) didn't inactivate p110β, nor did 100 nM of a scrambled peptide IYKTSKTRSQKTKIKPNT (SEQ ID NO: 5) (FIG. 51G). p85α preloaded with Selectide-18 failed to form a properly shaped p110β/p85α complex (FIGS. 51C and 51H); however, Selectide-18 did change p110α/p85α complex (FIGS. 511 and 51D).

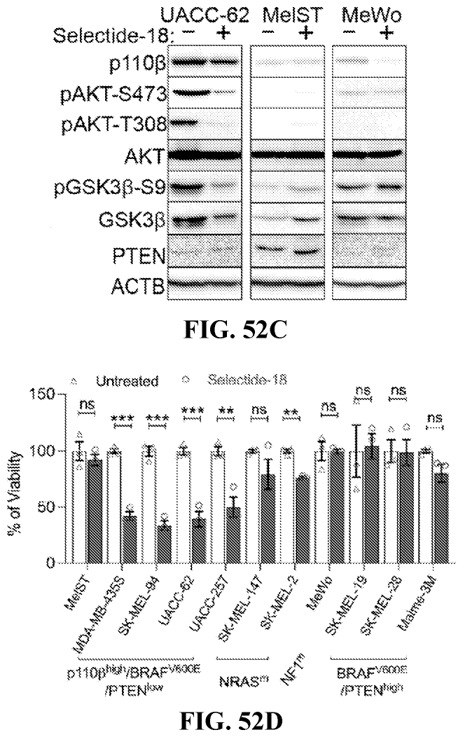

Fluorescent Selectide-18 was taken by SKMEL-147 cells 1 h after incubation and then decayed significantly in 96 hours (FIG. 52A). The half-life (HL) of Selectide-18 in SKMEL-147 or UACC-62 was 138 or 77 hours, respectively (FIG. 52B). Selectide-18 inactivated PI3K in p110β^{hyper} UACC-62 cells, but not in MelST or NF1m MeWo cells (FIG. 52C). This peptide suppressed the viability of p110β^{hyper} cells, without affecting that of other melanoma cells (FIG. 52D). Importantly, PIK3CBL535RMDA-MB-435S cells were sensitive to Selectide-18 (FIG. 52D), but not to AZD6482 (FIG. 49C). Selectide-18's IC₅₀ was 35 μM in UACC-62 cells, whereas IC₅₀s in MelST or MeWo cells were nearly 2 mM (FIG. 52E). This difference was not seen in AZD6482-treated cells (FIG. 49C), suggesting that Selectide-18 is more specific than AZD6482 to p110β hyper cells and perhaps active in suppressing p110β mutants. Moreover, Selectide-18 blocked the in vivo growth of UACC-62 xenografts (FIG. 52F). Hence, Selectide-18 is a novel and effective p110β drug.

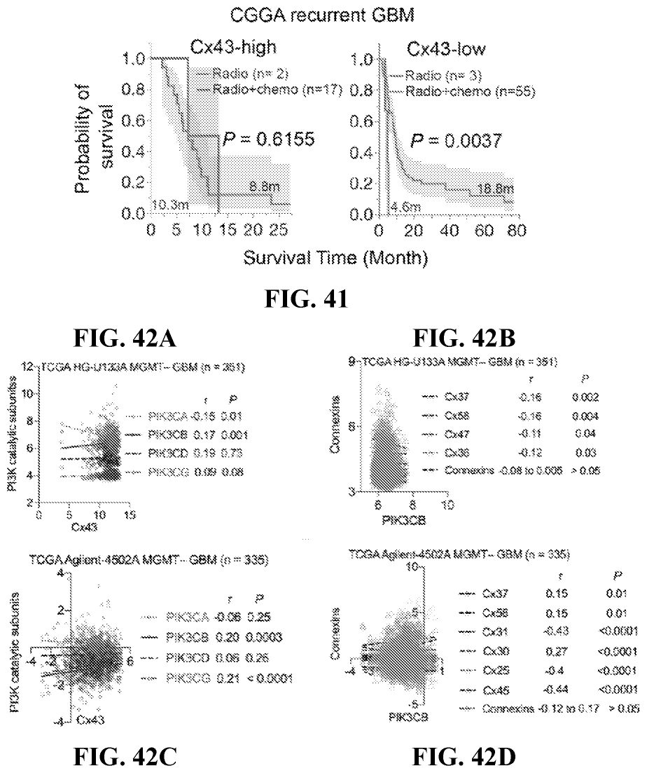

p110β^{hyper} melanoma accounted for 16.5% (21/127, FIG. 48B) to 40% (10/25, FIG. 48A; 4/10, FIG. 49A) of BRAFV⁶⁰⁰E cases, representing a considerable market (about 30% of BRAF^{V600E} melanoma) for p110β drugs such as Selectide-18. This thriving drug market can also be expanded to other p110β-dependent cancers—e.g., brain, breast, colon, lung, prostate, and metastatic tumors. This is in line with a predicted growth of the market for PI3K drug or peptide therapeutics according to researchandmarkets.com. In addition, BRAF^{V600E} and levels of p110β and PTEN proteins (FIG. 48A-48D) could be used as pharmacogenomic/proteomic markers for p110β therapies. AZD6482 and GSK2636771 are considered as competitors of Selectide-18. There are multiple clinical trials using these two drugs on solid tumors (NCT02215096, NCT04439188, NCT04439188, NCT03131908, NCT01458067, and NCT02615730). Only one of these trials is testing GSK2636771 in metastatic melanoma.

pan-PI3K inhibitors are non-selective and impose significant side effects. Applicant finds that p110β^{hyper} melanoma patients exhibit high risk of poor prognosis. Given that p110β is a survival factor for p110β^{hyper} melanoma, selectively targeting p110β is a feasible, innovative therapeutic option for these patients. Selectide-18, which mimics the β18 motif exclusively found in p110β and important for the assembly of p110β/p85 signaling complexes, is unique and different from current p110β-selective inhibitors. It is therefore innovative to harness p110 isoform-specific structures to develop p110 isoform drugs.

Without being bound by theory, Applicant believes that Selectide-18 is an actionable PI3K drug for melanoma and other cancers with PI3K involvement, particularly p110beta.

Determining PDs/PKs of Selectide-18 in p110βHyper Melanoma Cells Organoids and Maximum Tolerated Dose, Biodistribution, and Immune Response in Mice

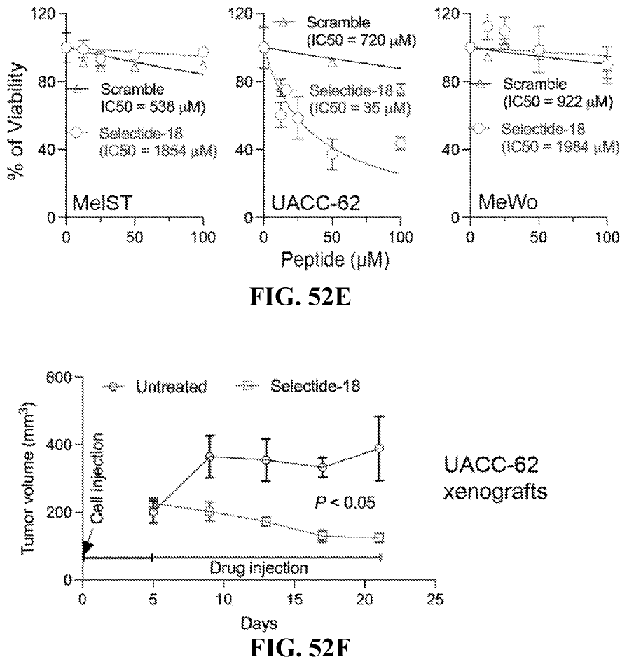

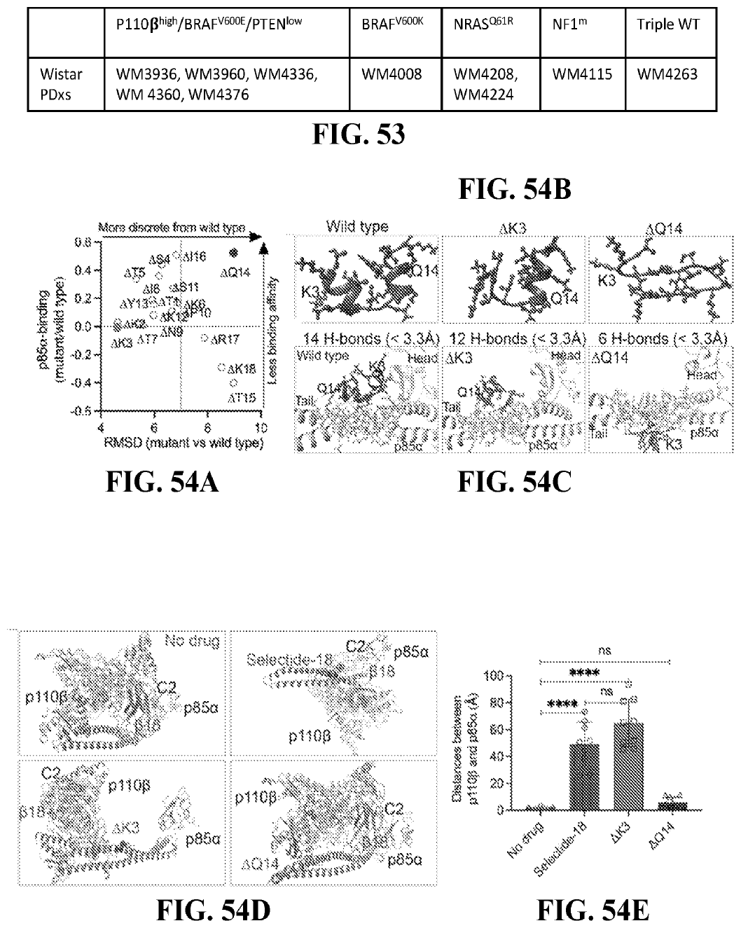

16.5% of Wistar PDXs were p110 hyper. Given that PDXs are more representative to original tumors than cell lines, Selectide-18 can be tested in PDXs (FIG. 53), which can be purchased from Rockland Immunochemicals Inc. Applicant therefore posits that p110β^{hyper} PDXs are sensitive to Selectide-18. The in vitro and in vivo PDs/PKs of Selectide-18 in p110β^{hyper} melanoma PDXs can be determined and can better represent original melanomas. As shown in FIG. 51A-51I, Selectide-18 mimics the β18 motif, where p85α binds. Hence, without being bound by theory, Applicant believes that Selectide-18 disassembles p110β/p85α to inactivate PI3K, which is supported by the results of in-silico analyses (FIG. 54A-54E). Based on the root-mean-square deviations (RMSDs), which measure distances between atoms, and p85α-binding affinities, deletion of Q14 (ΔQ14, FIG. 54A-54C) showed discrete 3D conformations from Selectide-18 and failed to block the assembly of p110β/p85α (FIG. 54D-54E), whereas ΔK3 exhibited no change. Analyses of Selectide-18 and its mutants can help reveal the model of action of this new p110β drug.

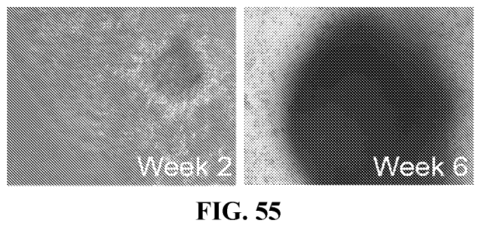

Cellular uptake, stability, and cytotoxicity of Selectide-18 can be determined. Wistar PDXs can be propagated in immunodeficient Nod scid mice. Primary PDX melanoma cells can be isolated using collagenase and cultured short-term (<10 passages) to maintain genetic profiles of the original tumors. Organoids mimic original tumors, offering advantages in testing Selectide-18. Methods for 3D culture of primary melanoma cells are well-established. Applicant has previous experience in 3D culturing (FIG. 55). (1) Time-lapse live cell imaging: Primary PDX melanoma cells can be cultured in Nunc Lab-Tek chamber slides (ThermoFisher). Organoids can be cultured in Matrigel. Cells and organoids can be treated with Alexa610-Selectide-18 (LifeTein) at various concentrations (0 to 100 μM). Alexa610 is used to trace peptides in live cells. Treated cells and organoids can be placed in the chamber with CO2 and a temperature control adapted to the CSU-X1 Nikon spinning-disk confocal system (FBRI imaging core). Z-stacks of Alexa610 fluorescence images can be recorded at different time points (1, 2, 4, 8, 16, 24, 48, 96, and 144 hours). Fluorescence intensities from same cells can be quantified using Nikon's NIS-Elements software to calculate peptide's half-lives from same cells (FIG. 52A-52B). (2) ELISA: PDX cells and organoids can be treated with Biotin-Selectide-18 (LifeTein) and then lysed. Biotinylated peptides can be quantified using HRP-conjugated streptavidin in an ELISA assay to determine peptide half-lives, which can be compared to imaging results. (3) PI3K activity assays: PDX cells and organoids can be treated with Scramble or Selectide-18 at different doses or times. PI3K activity can be analyzed by immunoblotting of pAKT and pGSK30 or ELISA of membrane lipids PIP2/PIP3 (Echelon Biosciences). (4) Cytotoxicity assays: Cytotoxicity can be measured using MTS viability assay, CytoTox 96® cytotoxicity assay (Promega), AnnexinV staining (BD Biosciences) followed by flow cytometry, and TUNEL assay (Abcam).

The mode-of-actin of Selectide-18 can be determined in p110β^{hyper} melanoma PDXs. The following peptides can be synthesized: Scramble, Selectide-18, ΔK3, and ΔQ14. These FIG. 13A-13B. 3D culture of tumor cells. peptides can be biotinylated or fluorophore-labeled (LifeTein). The following experiments can be performed in two p110β^{hyper} PDXs to reduce workload. (1) In vitro pull-down: Physical binding between recombinant histidine-tagged p85α (Sigma) and the above biotinylated peptides can be determined as follows. Streptavidin- or histidine-conjugated magnetic beads (ThermoFisher) can be used to pull down biotinylated peptides or histidine-tagged p85α, respectively. Biotin and histidine are controls. Binding between p85α and Selectide-18 or its mutants can be detected using immunoblotting. Interactions can be verified using histidine-tagged recombinant p110β/p85α, p110α/p85α, or p110δ/p85α (Sigma). p110s can be pulled down using antibodies (Cell Signaling Technology). If Selectide-18 disassembles p110β/p85α without affecting other p110s/p85α complexes can be determined using the following. (2) Surface plasmon resonance: To verify in vitro interactions between Selectide-18 and p85α, surface plasmon resonance that allows detection of weak interactions between a ligand and an analyte can be used. Biotin or biotinylated peptides can be pre-immobilized on the surface of a sensor chip using a Biotin-CAPture kit (Cytiva). By injecting histidine or histidine-tagged p85α at various concentrations, resonance units can be recorded using Biacore 3000 (FBRI), and dissociation constants (Kds) can be calculated using Biacore Insight Evaluation software. To determine whether Selectide-18 affects the stability of p110s/p85 complexes, histidine-tagged recombinant p85α protein (Abcam) can be immobilized on the chip using His Capture Kit (Cytiva). Recombinant p110s mixed with or without Selectide-18 or its mutants can be injected to determine Kds of p110s/p85α. (3) ELISA: To measure functional interactions between Selectide-18 and active p110β/p85α, p110α/p85α, or p110δ/p85α, p110s/p85α complexes can be preincubated with biotin-Selectide-18 or its mutants. A PI3K ELISA kit (Echelon Biosciences) can then be used to quantify ratios of PIP2:PIP3. (4) Time-lapse live cell imaging: Melanoma cells can be transfected with pCMV-p110β-mCherry (red) and pCMV-p85α-GFP (green), and then cultured in Nunc Lab-Tek chamber slides. Selectide-18 can be conjugated with Alexa405 (blue, LifeTein) and added to these cells. Cells can be starved and then replenished with 0.1% FBS. Fluorescence images from two channels (mCherry and GFP, mCherry and Alexa405, or GFP and Alexa405) can be recorded by the CSU-X1 Nikon spinning-disk confocal system at 0.5, 1, 2, 4, 8, 16, 24, 48, and 96 hours. Time-lapse images/videos showing co-localization of p85α (green, represented in greyscale)/p110β (red, represented in greyscale), p110β (red, represented in greyscale)/Selectide-18 (blue, represented in greyscale), or p85α (green, represented in greyscale)/Selectide-18 (blue, represented in greyscale) can be made using Nikon's NIS-Elements software. Results can reveal physical binding, Kds, p110s' IC50s, and spatial/temporal changes of p110β/p85α complexes in living cells and thus the role(s) of individual residues.

The maximum tolerated dose, biodistribution, and immune response of/to Selectide-18 in mice can also be determined. In vivo PKs of Selectide-18 can be monitored in normal C57BL/6 mice (Jackson Laboratory). Mice can be topically treated on the shaved skin with Alexa610-Selectide-18 (mixed with 1.25% hydroxyethyl cellulose) one time at 0, 50, 100, and 200 μM (=1.5, 3, and 6 mg/kg). There can be 6 time points (1, 2, 4, 8, and 16 days). Maximum tolerated dose is determined based on weight loss (>10%) and other severe behavior changes. To determine biodistribution, three mice from each time point can be euthanized. Peptide-treated skins and organs such as brain, liver, spleen, heart, kidney, colon, lung, and peripheral blood can be collected. Alexa610 fluorescence in ex vivo slices of organs can be taken using the IVIS Lumina s5 (FBRI imaging core) and PKs of Alexa610-Selectide-18 can be determined by Image J and Graphpad. Alexa610-Selectide-18 in blood cells can be quantified by flow cytometry and peptide PKs can be calculated based on the percentage of Alexa610+ cells. Immune response can be phenotypically analyzed and quantified by flow cytometry. These immune cells include CD3+/CD8+ cytotoxic T cells, CD3+/CD4+/FoxP3+ regulatory T cells, CD11b+/CD11c+/MHCII/CD80+/CD86+ dendritic cells, CD11c−/CD11b+/F4/80+/iNOS+M1 macrophage, CD11c−/CD11b+/F4/80+/CD163+/CD200R+M2 macrophage, and CD11b+/Gr-1+ myeloid-derived suppressor cells.

Without being bound by theory, it is believed that half-lives of Selectide-18 in PDX cells and organoids can be equivalent to those in cell lines (FIG. 52B) and Selectide-18 can suppress the viability of p110β^{hyper} PDXs. Selectide-18 can be enriched and stable in the skin, but not in other organs and blood. This peptide will not induce unfavorable immune response in normal mice.

### Determining the In Vivo Efficacy of Selectide-18 and Immunotherapies

While Applicant's in vivo results show that Selectide-18 alone slows down tumor growth in mice (FIG. 52F), combination therapies can also be evaluated. Recently established effective melanoma treatments include immune check point inhibitors e.g., a combination of anti-PD-1 and anti-CTLA-4 antibody (>50% effective in clinic) or adoptive T cell therapy e.g., chimeric antigen receptor (CAR)-T cell therapy (6 ongoing trials using CAR-T cells based on clinicaltrials.gov). However, not all melanoma patients at advanced stages exhibit outstanding responses to immunotherapies and patients do become relapsed. This is at least in part due to hyperactivated PI3K signaling which often causes resistance to immunotherapies. p110s, particularly p110δ, is important for immune surveillance in cancer and p110δ inhibition may induce unfavorable immune responses. However, the likelihood for toxicity induced by p110β inhibition is remote because p110β is not required for PI3K activation in hematopoietic cells. Given that Selectide-18 can be selective in blocking p110β and specific for p110βhyper melanoma (FIGS. 51A-51I and 52A-52F), this peptide medicine is less likely imposes negative effects. There is only one ongoing clinical trial (NCT03131908) testing the combination of GSK2636771 and pembrolizumab (anti-PD-1 antibody) in metastatic melanoma. No trials currently investigate PI3K drugs and CAR-T therapies in melanoma. It is therefore imperative to determine the efficacy of Selectide-18 and immunotherapies in this cancer. Without being bound by theory, Applicant believes that Selectide-18, which deactivates PI3K, effectively slows down the in vivo growth of p110β^{hyper} melanoma together with immunotherapies.

The therapeutic effect/immune response can be evaluated in a genetically-engineered mouse (GEM) melanoma model. A GEM line B6.Cg-Tg(Tyr-cre/ERT2)13Bos Braf^{tm1Mmcm}Pten^{tm1Hwu}/BosJ purchased from Jackson Laboratory can be maintained, which has previously been used in testing immunotherapies. This line is hemizygous for the Tg(Tyr-cre/ERT2)13Bos transgene, in which the mouse tyrosinase promoter/enhancer is placed upstream of a tamoxifen-inducible CreERT2 fusion gene, thereby permitting a tamoxifen-induced expression exclusively in melanocytes. This line carries heterozygous Braf^{tm1Mmcm} allele and homozygous floxed Pten^{tm1Hwu} allele that result in Braf^{V600E}/Pten^{null} melanoma after topical application of tamoxifen. 2-month-old mice can be topically treated with 4-hydroxytamoxifen (4-HT) and autochthonous melanomas form. When tumors are visible, mice can be randomly grouped and treated as follows: (1) Scramble; (2) Selectide-18; (3) mouse CAR-T cells; (4) ipilimumab and nivolumab; (5) Scramble and mouse CAR-T cells; (6) Scramble and ipilimumab/nivolumab; (7) Selectide-18 and mouse CAR-T cells; and (8) Selectide-18 and ipilimumab/nivolumab. Each treatment group can have 10 mice based on Statistical Plan (see below).

Treatment schedule: For peptides, a clear gel constituted with 1.25% hydroxyethyl cellulose and 100 μM (=3 mg/kg) peptide can be prepared and topically apply the gel to the skin lesions daily. This approach has been used in converting a Cx43-mimetic peptide into an FDA-approved drug for skin ulcers. For immune checkpoint inhibitors, the combination of the anti-CLTA-4 antibody ipilimumab and the anti-PD-1 antibody nivolumab can be tested because this combo is better than individual treatment. These antibodies can be purchased from Selleckchem. 0.3 mg/kg of drugs can be intraperitoneally injected biweekly. Mouse CAR-T cells can be produced in the Ma lab at the CHOP. Active CD8+ mouse T cells transduced with retroviruses encoding a murine anti-TRP1 CAR derived by fusing an anti-TRP1 scFv with an N-terminal Myc tag to the CD8α transmembrane domain, CD28/41BB costimulatory domain, and finally the CD3ζ intracellular domain. CAR expression can be verified by the Myc tag. 10⁷ mouse CAR-T cells can be injected through tail vein. Tumors can be measured daily and tumor volume can be calculated (length×width²/2). Mice can be euthanized when the tumor diameter reaches 1.5 cm. If no tumor or the tumor diameter is less than 1.5 cm, mice can be analyzed for another two to three months. Tumor recurrence or regrowing due to therapy-resistance has been observed in this GEM model.

The Bliss Independence combination model can be used to evidence synergy. Immune responses can be monitored in tissues collected. Different immune cells from tumor, peripheral blood, lymph nodes can be characterized by flow cytometry (see previous). CAR-T cells can be sorted as CD3+/Myc+/PD1+/TIM3+. Expression of p110s, pAKTs (PI3K activity), Ki67 (proliferation), and cleaved caspase-3 (apoptosis) in tissues can be determined using immunohistochemistry or immunoblotting. The tumor size can be analyzed using parametric/non-parametric approaches (e.g., two-sample t-test or Welch approximation with equal or unequal variances and Wilcoxon Rank-sum test). For time-to-event data, the Kaplan-Meier approach with the log-rank test can be used. 10 mice per group can achieve 80% power to detect an effect size of 1.5 given a significance level of 0.05 using a two-sided, two-sample t-test. Potential confounding risk factors can be evaluated by a multivariable regression modeling approach.

The therapeutic effect/immune response can be evaluated in a melanoma xenograft model. One p110β^{hyper} melanoma PDX (FIG. 53) that effectively responds to Selectide-18 based on results from above can be inoculated in 6-8 weeks old immunodeficient NOD-Prkdcscid IL2rgtm1/Bcgen mice (Biocytogen). These mice lack mature T cells, B cells or functional NK cells, and display cytokine signaling deficiencies after being reconstituted with human CD34+ peripheral blood mononuclear cells, which allow us to test human immune response. Similar models have been used for melanoma or other cancers. When tumors are about 200 mm³, mice can be treated as previously described. 10⁷ CAR-T cells can be used per mouse. Each treatment group can have 10 mice. Mice can be further analyzed as previously described.

Without being bound by theory it is believed Selectide-18 can increase levels of CD8+ T cells and decrease levels of CD4+ T cells, myeloid-derived suppressor cells, and macrophages in the tumor. Further it is believed that Selectide-18 can significantly inhibit the formation and progression of melanoma in combination with immunotherapies.

PIK3CB in Melanoma and Selective Inhibition with Selectide-18

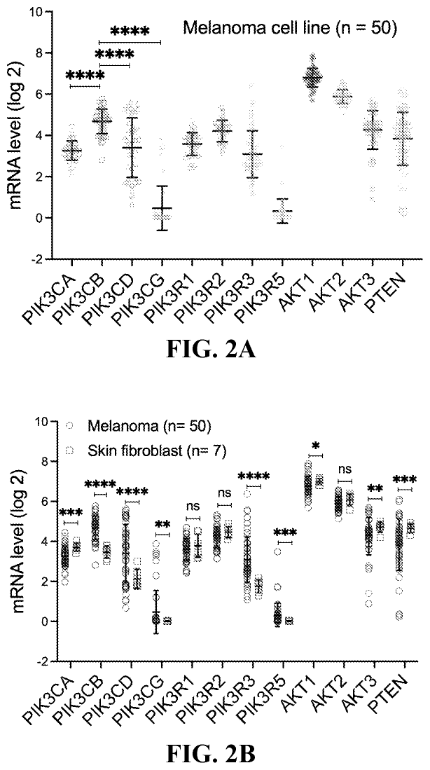

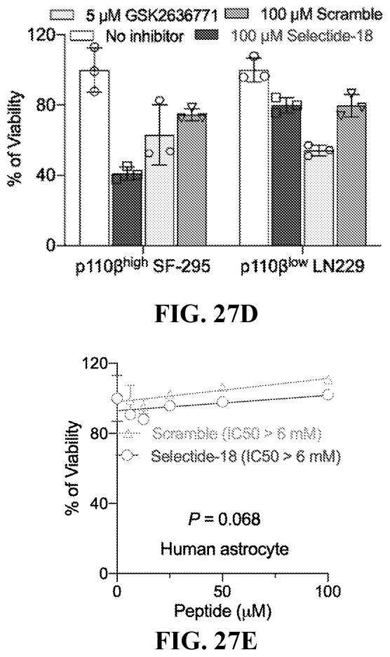

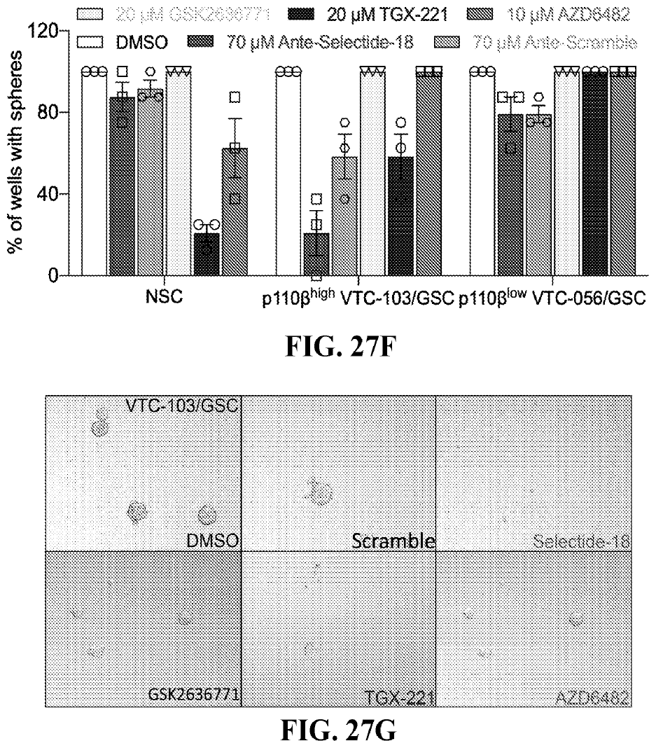

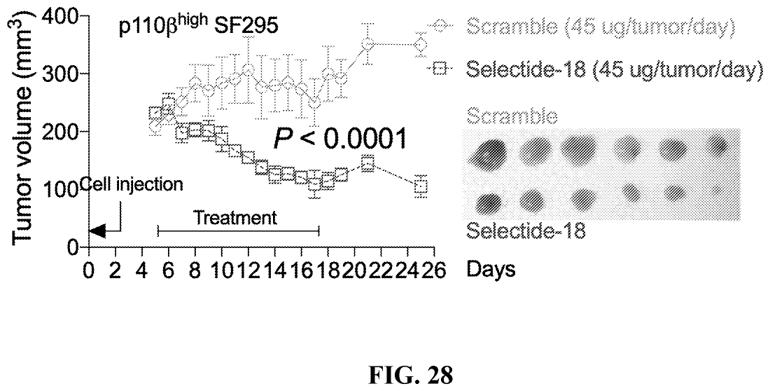

PI3KC isoforms are differentially expressed in melanoma (FIGS. 2A2E). Treatment of various melanoma cell lines with non-selective PI3K and isoform selective inhibitors in was done. Results are shown in FIG. 22-23. Melanoma cell lines were also treated with Selectide-18 and viability measured. Results are shown in FIG. 24.

### Example 3—Connexin 43 Confers Chemoresistance Through Activating PI3K

### Introduction

Overcoming resistance to chemotherapy such as temozolomide (TMZ) has proven perplexing and remains a key unmet clinical need. As an alkylating agent, TMZ reacts with DNA at multiple sites, yielding O⁶-methylguanine lesions that subsequently induce DNA breaks and eventually cell death 1. Given that TMZ is able to pass the blood-brain barrier², this drug has been used as the frontline chemotherapy for glioblastoma (GBM) an aggressive and lethal cancer that accounts for approximately half of all malignant brain tumors and has a grim prognosis with an average survival time of 14.6 months^{3,4}. Adding to this dismal outcome, nearly 90% of patients with GBM succumb to tumor recurrence and the average survival for recurrent GBM is about 5.5-7.5 months due to limited therapeutic options and resistance to TMZ 5. Hence, overcoming TMZ resistance is key to effectively treating GBM and curbing GBM progression. Poor responses of nearly 50% of GBM patients to TMZ are due to the expression of O-6-methylguanine-DNA methyltransferase (MGMT)^{6,7}. MGMT repairs TMZ-induced DNA damage, conferring MGMT-dependent TMZ resistance; as such, inhibiting MGMT has shown encouraging clinical benefits⁸. Patients with no MGMT expression also develop MGMT-independent resistance to TMZ^{9,10}. Factors involved in MGMT-independent TMZ resistance include the DNA mismatch repair pathway and genetic alterations^{11,12} However, targeting these factors to circumvent TMZ resistance has been a daunting task. Deeper insights into MGMT-independent TMZ resistance are therefore needed.

Recently, several lines of evidence have indicated that the gap junction protein connexin 43 (Cx43; also known as gap junction protein A1, GJA1), a channel-forming protein important for intercellular communication¹³, controls the response of GBM cells to TMZ. Ectopic expression of Cx43 renders GBM cells resistant to TMZ¹⁴⁻¹⁷ and blocking Cx43 using different approaches such as antibodies or channel inhibitors restores TMZ sensitivity¹⁴⁻²⁰ However, it remains unclear whether Cx43-mediated TMZ resistance depends on MGMT. Our recent work²¹ reveals that high levels of Cx43 in MGMT-deficient GBM cell lines and primary patient samples correlate with poor responses to TMZ and that αCT1, a clinically-tested therapeutic peptide that comprises the Cx43 carboxyl terminus (CT) and an antennapedia cell-penetrating sequence²², antagonizes TMZ resistance. Nonetheless, the molecular underpinnings of Cx43-mediated TMZ resistance remain elusive, making it difficult to effectively target Cx43 to treat GBM.

In this Example, Applicant demonstrates the role of connexins in GBM prognosis and TMZ resistance, explored how Cx43 activates phosphatidylinositol-3 kinase (PI3K) independent of Cx43 channels and induces TMZ resistance, and examined a candidate triple combinational therapy entailing the Cx43 inhibitor αCT1, PI3K-selective inhibitors, and TMZ in preclinical studies for its effectiveness in overcoming TMZ resistance.

### Materials/Subjects and Methods

### Reagents

TMZ (AbMole BioScience), GSK2636771 (AdooQ Bioscience), TGX-221 (AdooQ Bioscience) were reconstituted in dimethyl sulfoxide (DMSO) at a concentration of 50-80 mM. αCT1 and Gap27 were purchased from LifeTein, LLC. Lyophilized peptide was reconstituted in 1×PBS (137 mM NaCl, 2.7 mM KCl, 10 mM Na₂HPO₄, and 1.8 mM KH₂PO₄) at a concentration of 5 or 10 mM. Puromycin was purchased from Millipore-Sigma and dissolved in sterile water at a concentration of 5 mg/ml. All chemicals were aliquoted (to avoid repeated freeze/thaw cycles that decrease drug activity) and stored at −80° C.

### Cell Culture

GBM cell lines, primary GBM cells, glioblastoma stem cells (GSCs), and human astrocytes were cultured as previously described²³. Cell lines have been authenticated by the ATCC authentication service utilizing Short Tandem Repeat (STR) profiling. Primary cells VTC-001, VTC-003, VTC-005, and VTC-103 were cultured in DMEM supplemented with 15% fetal bovine serum (Peak Serum, Inc.) and penicillin/streptomycin. Normal human astrocytes were cultured in MCDB-131 medium (Sigma) containing 3% fetal bovine serum (Peak Serum, Inc.), 10×G-5 Supplement (Gibco), and penicillin/streptomycin. Primary GBM cells were kept at low passages (no more than 10).

### Analysis of Online Databases

GBM gene expression datasets (cDNA microarrays or RNA sequencing) or the data of reverse phase protein assay (RPPA) associated with corresponding clinical information and protein immunostaining data of human tissues are downloaded from the following websites: (1) The Cancer Gene Atlas (TCGA) datasets: https://www.cbioportal.org and https://gliovis.shinyapps.io/GlioVis/; (2) Gravendeel, Rembrandt, Lee Y, and Murat GBM: https://gliovis.shinyapps.io/GlioVis/; (3) The China Glioma Gene Atlas (CGGA) datasets: https://gliovis.shinyapps.io/GlioVis/; (4) GBM cell lines from the Cancer Dependency Map (DepMap): https://depmap.org/portal/; (5) The Human Protein Atlas (THPA): https://www.proteinatlas.org. The Kaplan Meier survival analysis or the Cox hazard proportional model were used to determine the relationship between gene expression levels and patient survival. To determine the expression correlation between different genes or proteins, the Pearson correlation coefficient was calculated using GraphPad Prism 8 software.

### MTS Cell Viability Assay

Cell viability was determined by the MTS cell viability assay (Promega) as described previously 21,24-27. In brief, 250 to 1,000 cells were plated in the wells of a 96-well plate-based upon the cell growth rate. Because the drug treatment usually takes 6-7 days, fast-growing cells could be over-grown if plated at a high cell density. For αCT1 treatment experiments, Applicant intended to plate cells at a low density to minimize the formation of gap junctions, and thus, more Cx43-hemichannels can be present. Because the half-life of αCT1 is about 48 hours, cells were replenished with fresh αCT1 every other day, without replenishing other drugs. Cells were treated with vehicle (DMSO) and chemical inhibitors at the indicated doses. After 6 days MTS reagent was added to the cells to a final dilution of 10% and incubated at 37° C. for a 4-hour period. At each hour time point, the absorbance at 490 nm was measured using a FilterMax F3 microplate reader (Molecular Devices, LLC) according to the manufacturer's instructions. Percent cell viability was obtained by dividing the absorbance of treatment groups to those of untreated and respective vehicle control groups.

### Caspase 3/7 Activity Assay

Apoptosis was measured using the Caspase-Glo® 3/7 Assay (Promega) based on the manufacturer's instructions and Applicant's previous work. In brief, VTC-001 and VTC-103 cells were plated at 1,000 cells/well in 96-well plates and treated with drugs as described for 6 days. After 6 days, 100 μL of Caspase-Glo® reagent was added to each well and incubated at room temp (RT). The luminescence was measured using a FilterMax F3 microplate reader (Molecular Devices, LLC) according to the manufacturer's instructions.

### Immunoblotting

Immunoblotting was performed as described previously^{28,29}. Antibodies were purchased from Cell Signaling Technology (CST), Millipore-Sigma (MS), and SantaCruz Biotechnology (SC). Antibodies were diluted as follows: anti-phospho-Cx43-S368 (CST-3511, 1:1,000), anti-Cx43 (CST-3512, 1:1,000), anti-phospho-AKT-S473 (CST-4051, 1:1,000), anti-phospho-AKT-T308 (CST-4056, 1:1,000), anti-AKT (CST-4685, 1:1,000), anti-phospho-cRAF-5338 (CST-9247, 1:1,000), anti-phospho-ERK-T202/T204 (CST-4377, 1:1,000), anti-phospho-SRC-T4160 (CST-2101, 1:1,000), anti-p110α(CST-4249, 1:1,000), anti-p110β(CST-3011, 1:1,000), anti-p110δ(CST-34050, 1:1,000), anti-p85 (CST-4292, 1:1,000), anti-β-actin (MS-A3854, 1:50,000), and anti-GAPDH(SC-25778, 1:1,000).

### Co-Immunoprecipitation

Co-immunoprecipitation was performed as previously described²⁹. Cell pellets were lysed in lysis buffer containing 20 mM HEPES pH 6.8, 140 mM NaCl, 2.5 mM MgCl₂, 2.5 mM CaCl₂, 1% NP40, 0.5% sodium deoxycholate, protease inhibitor (Millipore-Sigma, MS), and phosphatase inhibitors (MS). Total protein lysates were divided equally for each IP with input and IgG controls. Samples were incubated with primary antibodies overnight at 4° C. Antibodies were diluted as follows: anti-Cx43 (MS-C6219, 1:50), anti-p110α(CST-4249, 1:25), anti-p110β(CST-3011, 1:25), anti-p110δ(CST-34050, 1:25). All antibodies were from Rabbit thus Rabbit IgG (SC-2027, 1:400) was used as a control. Samples were then incubated at RT for 1 hour with Protein G Dynabeads™ (Thermo-Fisher). Protein-bead complexes were washed 3× with lysis buffer. The precipitated proteins were run on a 15% SDS-PAGE gel.

### Gene Knockdown or Expression

Knockdown of Cx43 or PI3K genes was described previously²⁴. Short hairpin (sh) RNA of Cx43 (TRCN0000059773), previously verified was purchased from Millipore-Sigma. shRNAs previously verified for PI3K genes were purchased from Thermo-Fisher Scientific (PIK3CA: RHS4844-101656239; PIK3CB:RHS4884-10165656350; PIK3CD:RHS4884-101655755). pBABE-PIK3CA-E545K, pCMV5-ERK2-L73PS151D, and pBABE-SRC-Y527F were purchased from Addgene. Transfection and expression of these plasmids were described previously²⁶.

### ATP/Glutamate Release

ATP release was measured using the Kinase-Glo© Luminescent Kinase Assay (Promega) as per the manufacturer's instructions. Glutamate release was measured using the Amplex™ Red Glutamic Acid/Glutamate Oxidase Assay Kit (ThermoFisher) according to the manufacturer's instruction.

### Bliss Independence Model

Synergism was assessed by Bliss independence^{30,31} which is based on the null hypothesis that each drug acts independently without super-additive or antagonistic effects. Applicant calculated the predicted additive effect using Effect(a+b)=Ea+Eb−EaEb for dual therapies and Effect(a+b+c)=Ea+Eb+Ec−EaEb−EaEc−EbEc−EaEbEc for triple therapies. Synergistic combinations resulted in higher than predicted values, antagonistic combinations had lower than predicted values, and additive combinations were equal to predicted values. The overall effect was determined at each dose using excess over Bliss (EOB) scores. EOB>0% indicated a synergistic effect, EOB=0% indicated an additive effect, and EOB <0% indicated an antagonistic effect.

### Mouse Experiments

Mouse experiments were performed based on the methods described previously^{24,26,32}, with modifications. All animal studies were approved by the Institutional Animal Care and Use Committee of Virginia Tech. 2×10⁶ SF-295 cells were mixed with Matrigel© Matrix (Corning) and subcutaneously injected into the flanks of 8-week-old SCID/beige mice (Taconic Biosciences). 8 days post-injection, mice were treated with drugs as indicated in the figure. Drugs were administered every other day via intraperitoneal injection (TMZ and TGX-221) or through intratumoral injection (αCT1). Tumors were measured daily using a caliper. On day 18, mice were euthanized, and tumors were harvested. Tumor volumes (mm³) were calculated using the formula: (length×width²)/2.

### Statistical Analyses

One-way ANOVA with Dunnett test for correction of multiple comparisons, Fisher's exact test, and Student's t test were used to determine statistical significance.

### Results

Cx43, but not Other Connexins, is Highly Expressed in GBM and Correlates with Poor Prognosis and Chemoresistance

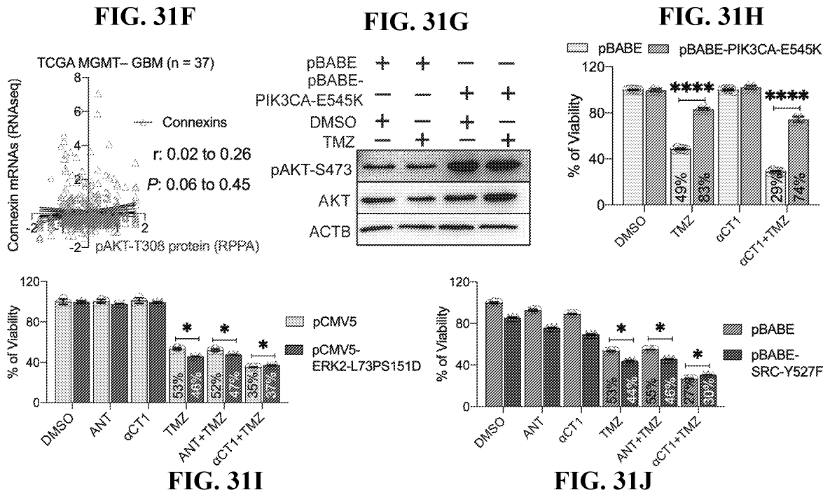

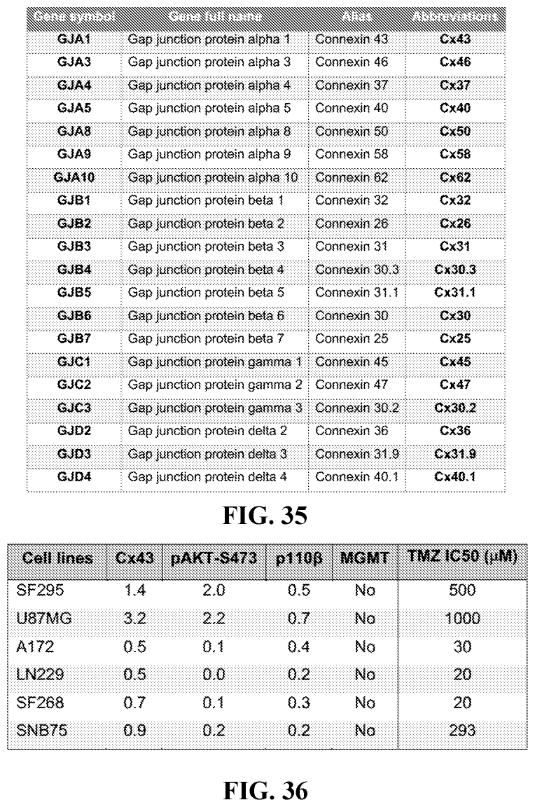

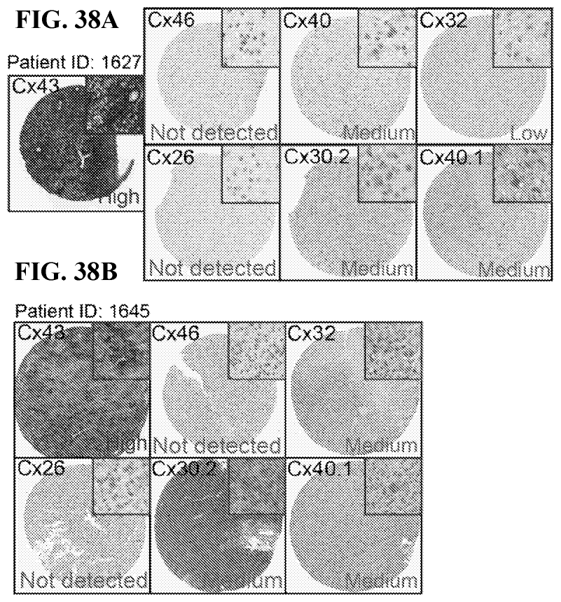

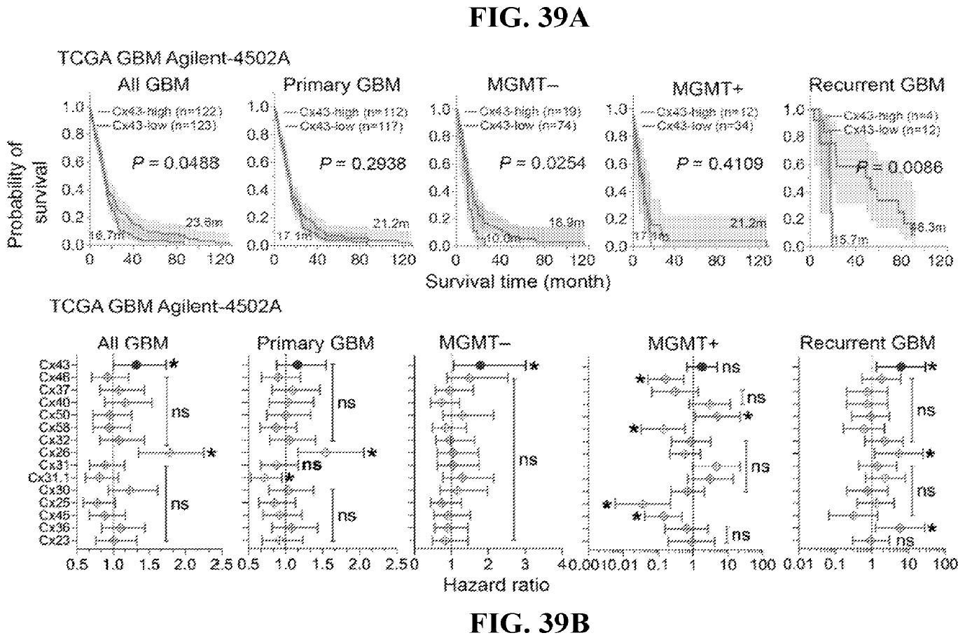

There are 21 known connexins (FIG. 35). Whether all these connexins are equally important for GBM survival and chemoresistance has not yet been explored. To address this, Applicant queried publicly available online GBM databases and analyzing programs, including The Cancer Genome Atlas (TCGA; https://www.cancer.gov/tcga), GlioVis³³, Chinese Glioma Gene Atlas (CGGA), and the Cancer Dependency Map (DepMap)³⁴. Cx43 mRNA was consistently expressed at the highest level among all connexins in primary GBM tumors from six different and 54 GBM cell lines datasets (FIG. 29A-29E and FIG. 37A-37D). Notably, despite that different connexins were detected in these studies, levels of Cx43 mRNA were significantly higher than other connexins (P<0.0001). Based on immunostaining results retrieved from The Human Protein Atlas³⁵, levels of Cx43 protein in high-grade glioma were also significantly higher than other connexins, except Cx37 or Cx40 (FIG. 29F). In Cx43-high tumors, other connexins were scored as either not detected, low, or medium in the same tumor (FIG. 29G and FIG. 38A-38B), suggestive of a dominant expression of Cx43. Collectively, Cx43 is expressed at the highest level among all connexins in GBM and high-grade glioma.

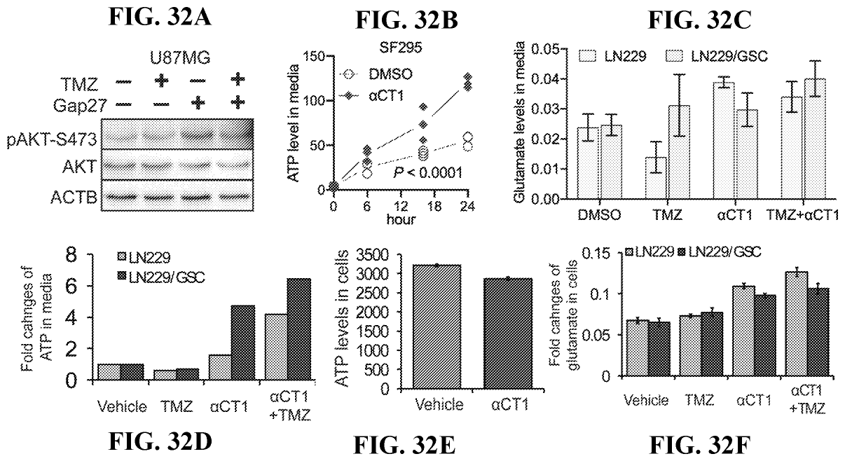

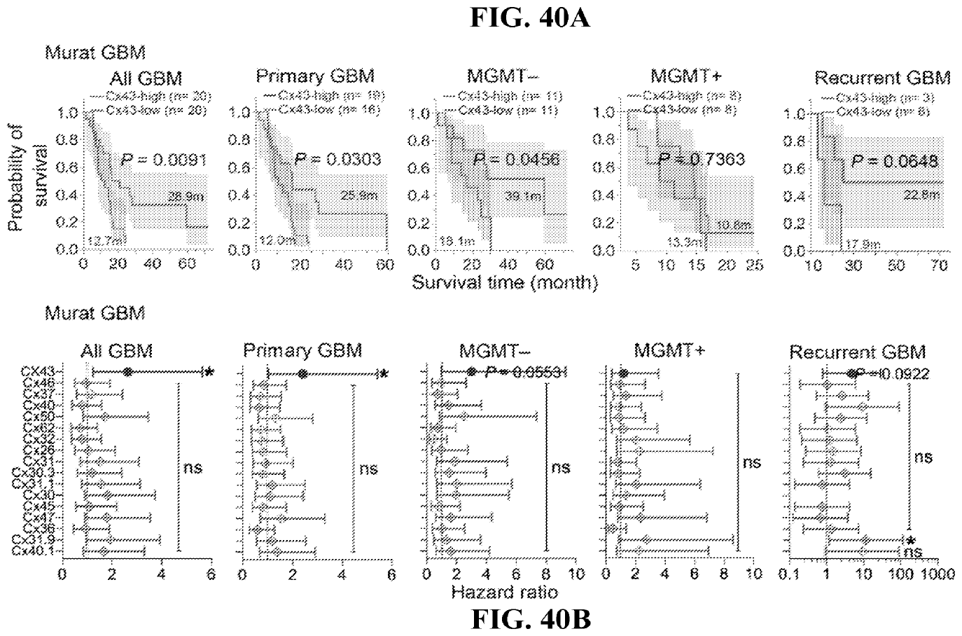

Kaplan-Meier analyses (FIG. 30A and FIG. 39A) revealed that high levels of Cx43 mRNA were associated with poor prognosis of GBM patients (All GBM). However, the lifespan of Cx43-high primary GBM patients was not significantly shorter than that of Cx43-low patients (Primary GBM, P >0.05). Because about 50% of primary GBMs exhibit promoter methylation of MGMT, designated as MGMT-⁶, and that Cx43 correlates with the survival of MGMT-patients 2, Applicant tested MGMT-primary GBMs, high levels of Cx43 correlated with poor prognosis (P<0.05), whereas Cx43 levels had no relationship with the survival of MGMT+(promoter unmethylated) GBM patients (P >0.05). It was not surprising that Cx43-high recurrent GBM patients exhibited a dismal prognosis (Recurrent GBM) because recurrent GBMs are often refractory to TMZ 5. Similar results were found in multiple GBM datasets (Supplemental FIG. 39A and FIG. 40A). To compare Cx43 with other connexins, Applicant performed Cox univariate analyses, which yield a hazard ratio (HR) that determines chance of death (HR >1 indicates high risk of death). Consistent with the results of Kaplan-Meier analyses (FIG. 30A), Cx43-high patients had considerably high HRs in the group of All GBM, MGMT−, and Recurrent GBM. In contrast, most of other connexins failed to display a notably high risk of death in all three groups (FIG. 30B and FIG. 39B and FIG. 40B), suggesting that Cx43, compared to other connexins, strongly correlates with poor prognosis of MGMT− GBM.

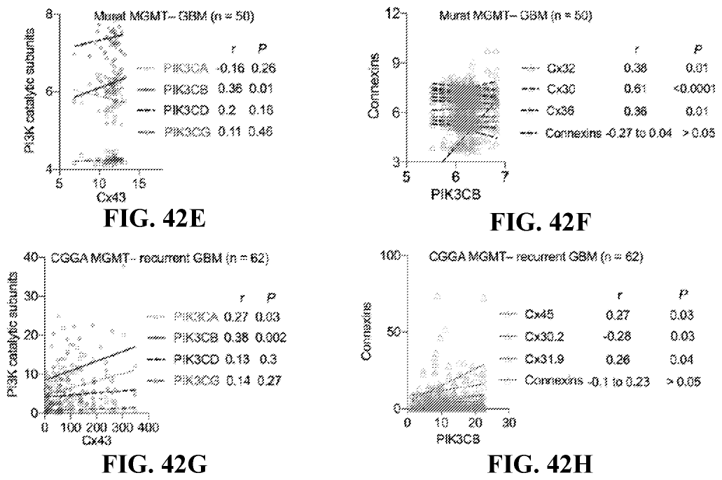

Previous research has demonstrated that TMZ improves prognosis of GBM patients when used in combination with radiation⁶. To determine how connexins contribute to this treatment regime, MGMT− GBM patients treated with radiation (Radio) were compared to patients treated with radiation and TMZ (Radio+TMZ) or radiation and chemo (Radio+chemo) (FIG. 30C and FIG. 41). While the addition of TMZ or chemo did increase the survival of both Cx43-high and Cx43-low patients, there was no statistically significant difference between these treatments in the Cx43-high group in three GBM datasets (P >0.05), suggesting that Cx43-high patients are resistant to TMZ. Of note, levels of Cx37, Cx47, or Cx31.9 did not consistently correspond to the risk of death in three datasets (FIG. 30D). Together, these results demonstrate that Cx43 is expressed at the highest level among all connexins in GBM and contributes to chemoresistance as well as poor prognosis of MGMT-deficient GBMs.

### Cx43 Confers Resistance to TMZ by Activating PI3K

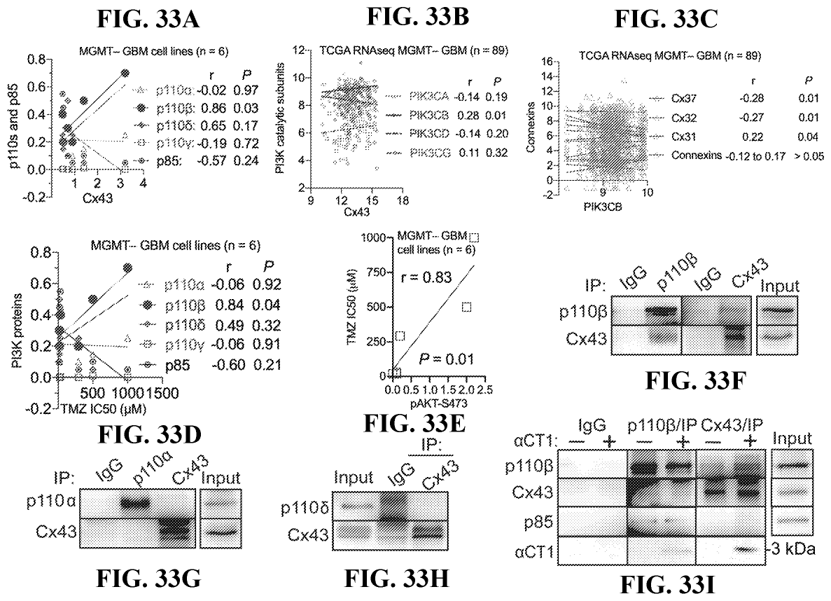

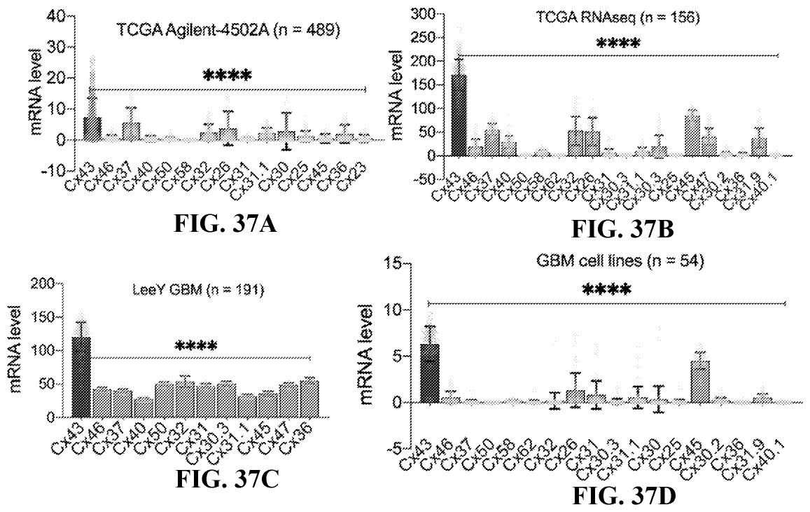

Next, Applicant explored how Cx43 confers TMZ resistance. Applicant has previously shown that the Cx43 peptide inhibitor αCT1 inactivates PI3K²¹. Without being bound by theory, Applicant believes that Cx43 activates PI3K to induce TMZ resistance. To test this, Applicant treated Cx43-high/TMZ-resistant U87MG cells with TMZ or αCT1. αCT1 blocked phosphorylation of Cx43 at serine 368 (FIG. 31A, pCx43-S368), a phosphorylation site critical for Cx43 activity³⁶. Aligning with Applicant's belief, αCT1 induced a 5-fold decrease of the phosphorylated form of AKT serine/threonine kinase (AKT; FIG. 31A, pAKT-S473), indicative of a strong inhibition of PI3K. Previous research^{37,38} has suggested that Cx43 regulates the activity of the mitogen-activated protein kinase (MAPK) pathway, including the RAF proto-oncogene serine/threonine-protein kinase (RAF)/extracellular-signal-regulated kinase (ERK) cascade and the SRC proto-oncogene non-receptor tyrosine kinase (SRC) pathway. αCT1 modestly reduced levels of pcRAF-5338, pERK-T202/T204, or pSRC-T416. Hence, αCT1 influences the activity of multiple signaling pathways. The Cx43-induced activation of PI3K was further verified by the knockdown of Cx43 using a short hairpin RNA (shRNA) because the Cx43 shRNA not only drastically decreased levels of Cx43 and pCx43-S368 but also remarkably mitigated PI3K activity in U87MG cells but not in Cx43-low/TMZ-sensitive A172 cells (FIG. 31B). Through reanalyzing data from previous work^{21,24} Applicant detected a strong correlation between Cx43 and pAKT-5473 in six MGMT-deficient GBM cell lines (FIG. 31C and FIG. 36). A positive trend was also found between levels of Cx43 mRNA and pAKT-S473 or pAKT-T308 in 37 MGMT-deficient GBM patients in the TCGA dataset (FIG. 31D). Other connexins, however, failed to show statistically significant correlations with either pAKT-S473 (FIG. 31E) or pAKT-T308 (FIG. 31F).

To determine whether PI3K is required for Cx43-induced TMZ resistance, Applicant overexpressed PIK3CA-E545K, a PI3K mutant that constitutively activates PI3K, in U87MG cells (FIG. 31G). PIK3CA-E545K counterbalanced the growth inhibition induced by TMZ or by a combination of TMZ and αCT1 (FIG. 31H). This counteraction was not seen in U87MG cells expressing an active mutant of ERK (ERK2-L73PS151D; FIG. 31I) or SRC (SRC-Y527F; FIG. 31J). These results suggest that, while Cx43 activates multiple signaling pathways such as PI3K, ERK, or SRC, only the activation of PI3K is important for Cx43 to induce TMZ resistance.

### Cx43 Selectively Binds to the PI3K Catalytic Subunit β and Activates PI3K

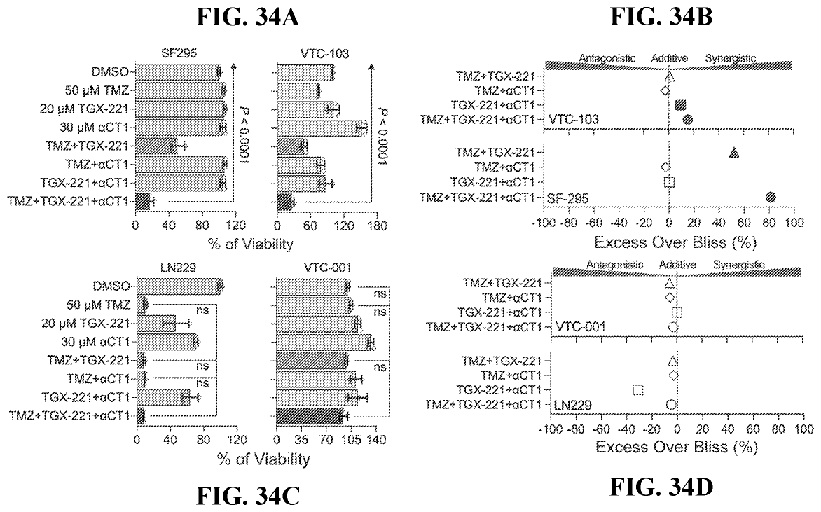

Because the Cx43-CT regulates the activity of Cx43-channels³⁹, without being bound by theory Applicant believes that small molecules such as ATP or glutamate released from Cx43-channels activate PI3K in GBM cells as they do in astrocytes⁴⁰. To test this, Applicant treated U87MG cells with Gap27, a Cx43 peptide inhibitor that targets the second extracellular loop of Cx43 and blocks Cx43-channels⁴¹. Gap27, however, did not attenuate PI3K activity (FIG. 32A). Next, Applicant examined the levels of ATP or glutamate in Cx43-high SF295, Cx43-low LN229, and Cx43-high LN229/GSC cells. Regardless of levels of Cx43, levels of ATP or glutamate in culture media either elevated or remained unchanged in αCT1-treated cells (FIG. 32B-32D), consistent with the dephosphorylation of Cx43 at S368 by αCT1 (FIG. 31A), which enhances the permeability of Cx43 hemichannels⁴². ATP or glutamate levels remained unchanged in cells (FIG. 32E-32F). These results suggest that Cx43-channels are dispensable for PI3K activation in GBM cells.

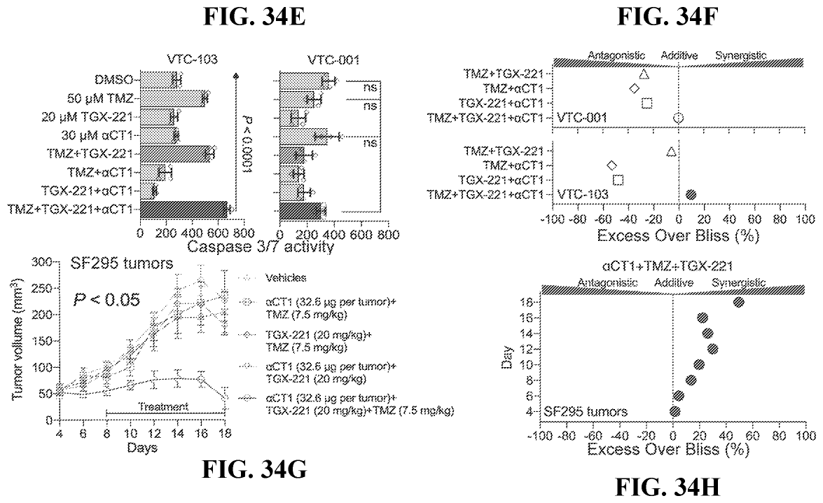

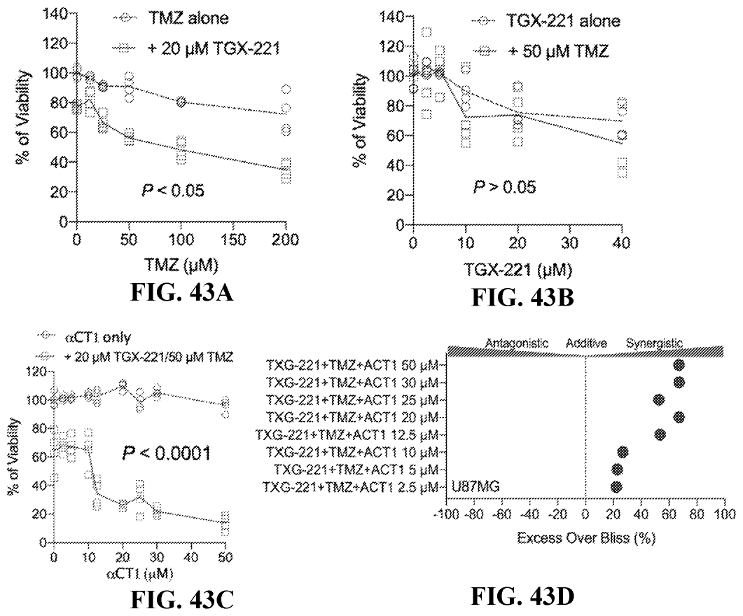

Cx43-CT interacts with certain signaling molecules³⁷. It is likely that Cx43 binds to PI3K catalytic subunits to activate PI3K. The Class I PI3K family consists of four highly homologous catalytic subunits: PI3K catalytic subunits α, β, δ, and γ (PIK3CA, PIK3CB, PIK3CD, and PIK3CG) encoding p110α, p110β, p110δ, and p110γ, respectively⁴³. Previous work has demonstrated that PI3K catalytic subunits play different roles in GBM cell survival, with p110β being the most dominant isoform in GBM²⁴. To determine whether PI3K catalytic subunits also function divergently in Cx43-induced PI3K activation, Applicant reanalyzed protein expression data in six MGMT− GBM cell lines (FIG. 36). Levels of Cx43 protein showed a positive and statistically significant correlation with those of p110β, but not other p110s or the regulatory subunit p85 (FIG. 33A). mRNA levels of Cx43 also positively corresponded with those of PIK3CB, but not other PI3K subunits, in 89 MGMT− GBM patients in the TGCA RNAseq dataset (FIG. 33B). In the same dataset, PIK3CB displayed no or negative correlation with the 21 other connexin family members, except Cx31 (FIG. 33C). Such a positive correlation between Cx43 and PIK3CB was recapitulated in multiple GBM datasets (FIG. 42A-42H) and further verified by the finding that high levels of pAKT-S473 or p110β, but not other p110s, correlated with low TMZ sensitivity indicated by the increase of TMZ IC50s (FIG. 33D-33E). To further probe the molecular details of Cx43-induced PI3K activation, Applicant monitored protein-protein interactions between Cx43 and p110 proteins. Cx43 was co-precipitated with p110β (FIG. 33F) but not with p110α or p110δ (FIG. 33G-33H), demonstrating a selective binding of Cx43 to p110β. Applicant did not examine p110y because p110y is not detectable in GBM²⁴. To determine whether αCT1 binds to Cx43 and/or p110β, Applicant treated U87MG cell lysates with αCT1 and found that αCT1 was pulled down together with p110β and Cx43 (FIG. 33I). In the presence of αCT1, more p110β was found in the Cx43 precipitate. Without being bound by theory, this might be because the Cx43 antibody is able to precipitate αCT1/Cx43/p110β (or αCT1/p110β) and Cx43/p110β protein complexes. Taken together, Applicant has defined a previously unknown non-channel activity of Cx43, through which p110β is selectively bound and perhaps activated thereafter in GBM.

A Combination of αCT1 and p110β-Selective Inhibitors Overcomes TMZ Resistance

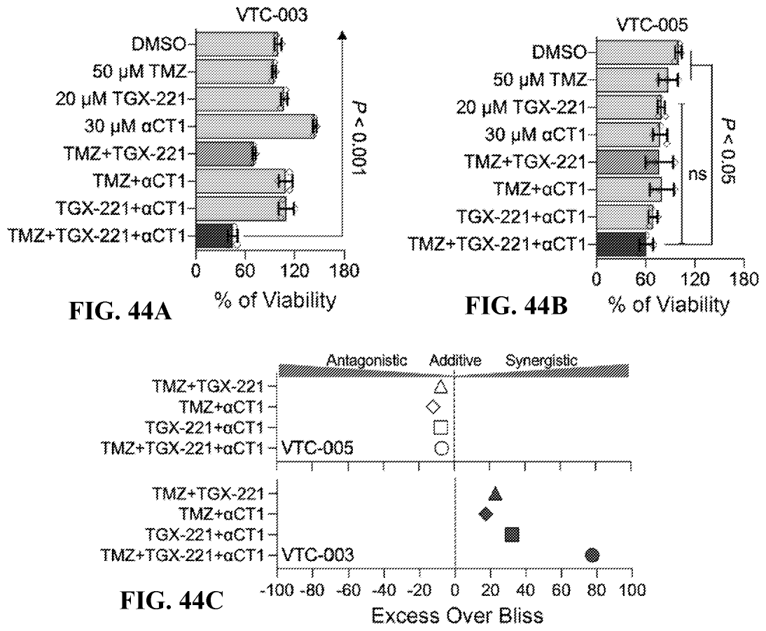

αCT1 alone increases the sensitivity of LN229/GSC xenograft tumors to TMZ²¹; however, the short half-life of αCT1 demands high concentrations and repeated drug delivery, which may limit its therapeutic potential. Prompted by the above results, Applicant tested the combination of αCT1 and p110β-selective inhibitors in cultured cells and in mice. To achieve a synergistic therapeutic effect of multiple drugs, Applicant optimized the dose of each individual drug in U87MG cells. By varying doses of TMZ or a p110β-selective inhibitor TGX-221, Applicant found that the double combination of 50 μM TMZ and 20 μM TGX-221 did not significantly inhibit the viability of U87MG cells (FIG. 43A-43B). However, the addition of αCT1 greatly increased the cytotoxic effect of the TMZ/TGX-221 double combination (FIG. 43C). Next, Applicant assessed synergistic drug effects using the Bliss independent model, a method commonly used to measure drug synergy^{30,31}. This model yields excess over Bliss (EOB) scores. EOB >0% indicates a synergistic effect; EOB=0% means an additive drug effect; EOB <0% refers to an antagonistic effect. 2.5 to 10 μM αCT1 only yielded a weak synergistic effect on cell viability, whereas 12.5 to 50 μM αCT1 exhibited a much stronger synergistic inhibition of cell viability, together with TMZ/TGX-221 (FIG. 43D).

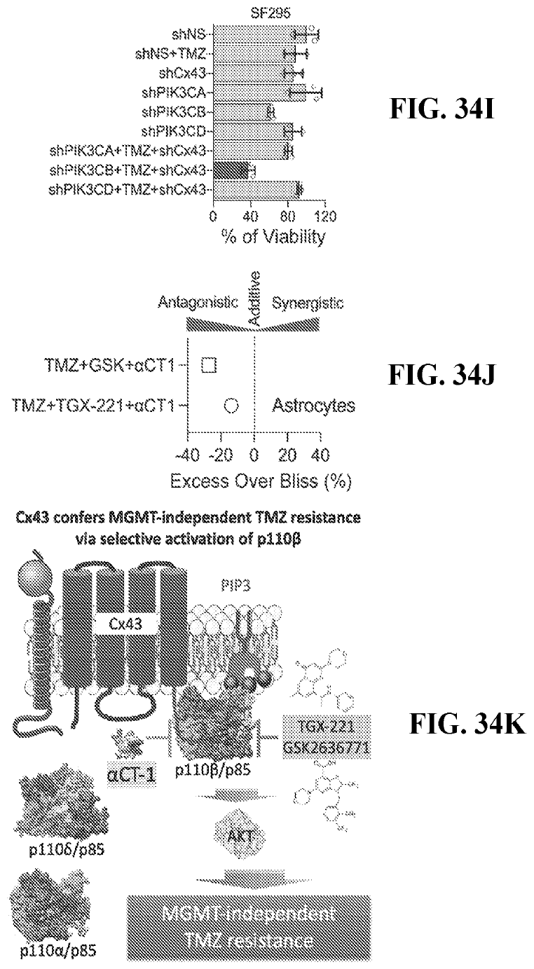

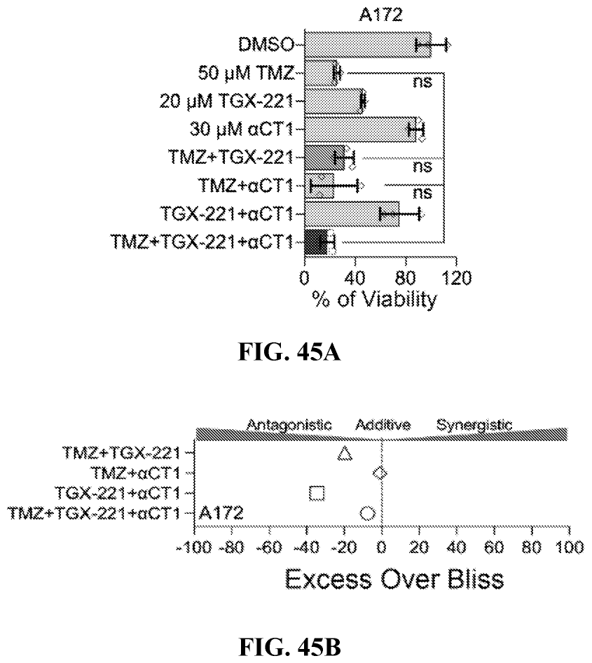

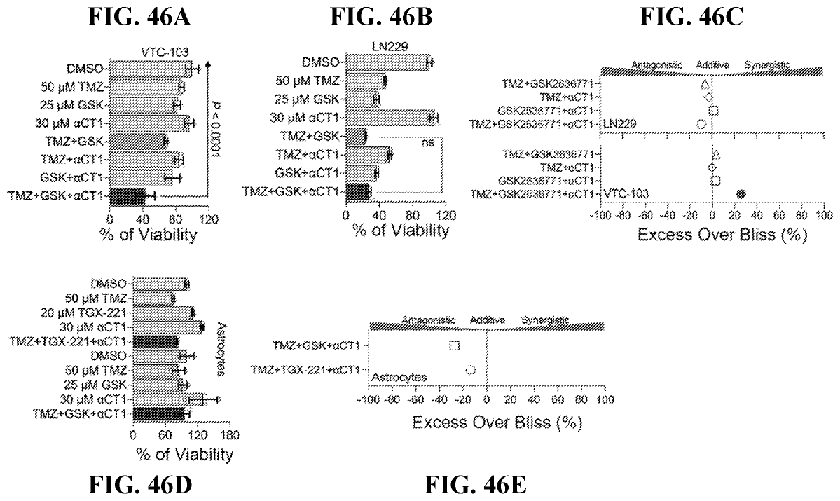

Based on these results, 30 μM αCT1, 20 μM TGX-221, and 50 μM TMZ were used in a triple combination named αCT1/TGX/TMZ combo. The αCT1/TGX/TMZ combo synergistically reduced the viability of MGMT−/TMZ-resistant SF295, VTC-103, and VTC-003 cells (FIG. 34A and FIG. 44A) that express high levels of Cx43 and p110β^{21,24} Notably, VTC-103, VTC-003, and other VTC lines described hereafter were derived from freshly dissected GBM tumors^{21,24}. EOB scores of the αCT1/TGX/TMZ combo were significantly higher than those of double combinations (FIG. 34B and FIG. 44C). This synergistic effect was, however, not found in MGMT−/TMZ-sensitive LN229 and A172 or MGMT−/TMZ-resistant VTC-001 and VTC-005 (FIG. 34C-34D, FIG. 44B-44C, and FIG. 45A-45B) whose levels of Cx43 and p110β are low^{21,24}. The αCT1/TGX/TMZ combo synergistically activated apoptosis in VTC-103 cells (FIG. 34E-34F), coinciding with the drastic decrease of cell growth (FIG. 34A), whereas apoptosis was not synergistically induced in VTC-001 cells. To verify the in vitro studies in vivo, Applicant treated mice bearing SF295 xenograft tumors with 7.5 mg/kg TMZ and 20 mg/kg TGX-221 through intraperitoneal injection in conjunction with 32.6 μg of αCT1 per tumor through intratumoral injection. The αCT1/TGX/TMZ combo (red line) stopped tumor growth (FIG. 34G, P<0.05), whereas double combinations exhibited limited to no inhibition. EOB scores of the triple combo increased over time, culminating on day 18 (FIG. 34H). This confirms a strong synergy amongst αCT1, TGX-221, and TMZ in vivo. To verify that the synergistic cytotoxicity is due to the blockade of Cx43/p110β, Applicant knocked down Cx43 and individual PI3K catalytic subunits using shRNAs. Depletion of p110β, but not p110α or p110δ, blocked the growth of SF295 cells (FIG. 34I), and only the combination of PIK3CB shRNA, Cx43 shRNA, and TMZ yielded synergistic inhibition of cell viability (FIG. 34J).

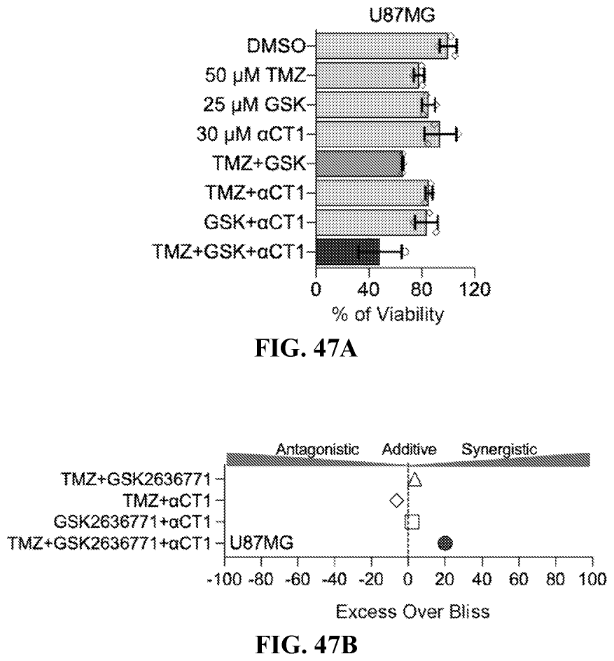

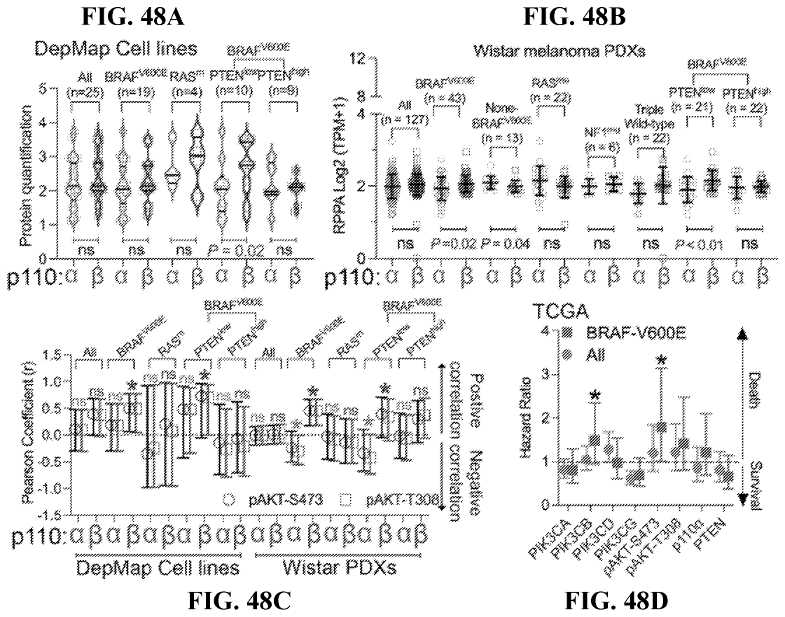

To corroborate results from TGX-221, Applicant tested another p110β-selective inhibitor GSK2636771 (abbreviated hereafter as GSK), which has been used in a clinical study⁴⁴. αCT1/GSK/TMZ combo entailing 25 μM GSK, 30 μM αCT1, and 50 μM TMZ synergistically blocked the viability of VTC-103 cells (FIGS. 46A and 46C) and U87MG cells (FIG. 47A-47B), but not the viability of LN229 cells (FIGS. 46B and 46C). αCT1/GSK/TMZ has achieved the same synergistic inhibition of GBM cell viability as the αCT1/TGX/TMZ combo. To determine the toxicity of these combinations on normal cells, Applicant treated astrocytes with αCT1/TGX/TMZ or αCT1/GSK/TMZ. These drug combinations did not increase TMZ alone-induced growth inhibition in astrocytes (FIGS. 46D and 46E), suggesting that addition of αCT1 and p110β-selective inhibitors does not exacerbate non-selective toxicity of TMZ to the normal brain. Collectively, these results demonstrate that simultaneously targeting Cx43 and p110β diminishes TMZ resistance.

### DISCUSSION

In this Example, Applicant has at least identified the molecular details underlying Cx43-induced MGMT-independent TMZ resistance. As illustrated in a model proposed in FIG. 34K, Cx43-CT binds to p110β/p85 signaling complex upon receiving signals from extracellular cues (i.e., growth factors). This selective binding brings the p110β/p85 signaling complex to the membrane and subsequently activates AKT. Activated PI3K/AKT signaling renders GBM cells resistant to TMZ, which is independent of MGMT. This model not only explains how a gap junction protein regulates chemoresistance through its non-channel activity but also provides a strong rationale for developing combinational therapies to overcome TMZ resistance. Indeed, the results shown in FIG. 34A-34K indicate that αCT1, a Cx43-CT mimetic peptide that likely blocks interactions between Cx43-CT and p110β, works synergistically together with p110β kinase inhibitors (directly blocking kinase activity) in overcoming TMZ resistance.

Prior studies report that approximately 20-60% of GBM patients express Cx43 mRNA and protein at high levels 15. In light of the fact that 45% of GBM patients express no MGMT^{6,7} there should be 10% (20%×50%) to 30% (60%×50%) of patients that are MGMT-deficient and express high levels of Cx43. Congruent with these results, Applicant has found that 16.7% of MGMT-deficient GBM patients express high levels of Cx43²¹. That being said, around 20% of Cx43-high GBM patients may be refractory to TMZ treatment in the clinic. Therefore, the combinational treatment developed herein can benefit MGMT-deficient/TMZ-resistant patients expressing high levels of Cx43, thereby having an important impact on future therapeutic intervention. Previous work has also revealed that, with the exception of Cx43, overexpression or inhibition of Cx30, Cx32, Cx26, or Cx46 also blocks the growth of rat or human glioma cells⁴⁵⁻⁵². However, contradictory to these results, other studies show that Cx30 and Cx32 have no effect on glioma growth^{48,53,54}. In line with the fact that Cx43 levels are much higher than other connexins in GBM and the finding that Cx43 controls chemoresistance, this connexin is therefore a prime therapeutic target for GBM.

Cx43 has long been considered as a tumor suppressor for glioma because overexpression of Cx43 leads to remarkable growth inhibition⁵⁵ and levels of Cx43 mRNA and protein inversely correlate with the aggressiveness of glioma⁵⁶. However, drawbacks in these studies have made the tumor-suppressive activity of Cx43 questionable. For example, while ectopically expressing Cx43 does inhibit tumor cell growth, it is unclear whether the loss of endogenous Cx43 in normal glial cells promotes gliomagenesis as other tumor suppressors do, namely p53 and NF-1. Nonetheless, it is possible that gap junction intercellular communication controlled by Cx43 is GBM suppressive because loss of this communication promotes oncogene-induced transformation 57. In contrast to these studies, a tumor-promoting role of Cx43 in GBM has been previously established. Cx43, whose mRNA levels are the highest among all connexins, not only correlates with GBM prognosis and chemoresistance but also activates PI3K independent of Cx43-channels to induce TMZ resistance. Therefore, it is likely that Cx43 has multifaceted roles in GBM: Cx43-channels inhibit GBM formation, whereas the Cx43 CT confers chemoresistance through activating PI3K, which is independent of Cx43 channel function, during GBM progression.

### REFERENCES FOR EXAMPLE 3

1 Tisdale, M. J. Antitumor imidazotetrazines-XV. Role of guanine 06 alkylation in the mechanism of cytotoxicity of imidazotetrazinones. Biochem Pharmacol 36, 457-462, doi:10.1016/0006-2952(87)90351-0 (1987).
2 Portnow, J. et al. The neuropharmacokinetics of temozolomide in patients with resectable brain tumors: potential implications for the current approach to chemoradiation. Clin Cancer Res 15, 7092-7098, doi:10.1158/1078-0432.CCR-09-1349 (2009).
3 Ostrom, Q. T. et al. CBTRUS Statistical Report: Primary Brain and Other Central Nervous System Tumors Diagnosed in the United States in 2011-2015. Neuro Oncol 20, iv1-iv86, doi:10.1093/neuonc/noy131 (2018).
4 Siegel, R. L., Miller, K. D. & Jemal, A. Cancer statistics, 2019. CA Cancer J Clin 69, 7-34, doi:10.3322/caac.21551 (2019).
5 Weller, M., Cloughesy, T., Perry, J. R. & Wick, W. Standards of care for treatment of recurrent glioblastoma—are we there yet? Neuro Oncol 15, 4-27, doi:10.1093/neuonc/nos273 (2013).
6 Stupp, R. et al. Radiotherapy plus concomitant and adjuvant temozolomide for glioblastoma. The New England journal of medicine 352, 987-996, doi:10.1056/NEJMoa043330 (2005).
7 Hegi, M. E. et al. MGMT gene silencing and benefit from temozolomide in glioblastoma. N Engl J Med 352, 997-1003, doi:10.1056/NEJMoa043331 (2005).
8 Quinn, J. A. et al. Phase II trial of temozolomide plus o6-benzylguanine in adults with recurrent, temozolomide-resistant malignant glioma. Journal of clinical oncology: official journal of the American Society of Clinical Oncology 27, 1262-1267, doi:10.1200/JCO.2008.18.8417 (2009).
9 Bocangel, D. B. et al. Multifaceted resistance of gliomas to temozolomide. Clinical cancer research: an official journal of the American Association for Cancer Research 8, 2725-2734 (2002).
10 Happold, C. et al. Distinct molecular mechanisms of acquired resistance to temozolomide in glioblastoma cells. Journal of neurochemistry, doi:10.1111/j.1471-4159.2012.07781.x (2012).
11 Cahill, D. P. et al. Loss of the mismatch repair protein MSH6 in human glioblastomas is associated with tumor progression during temozolomide treatment. Clinical cancer research: an official journal of the American Association for Cancer Research 13, 2038-2045, doi:10.1158/1078-0432.CCR-06-2149 (2007).
12 Messaoudi, K., Clavreul, A. & Lagarce, F. Toward an effective strategy in glioblastoma treatment. Part I: resistance mechanisms and strategies to overcome resistance of glioblastoma to temozolomide. Drug Discov Today, doi:10.1016/j.drudis.2015.02.011 (2015).
13 Palatinus, J. A., Rhett, J. M. & Gourdie, R. G. The connexin43 carboxyl terminus and cardiac gap junction organization. Biochim Biophys Acta 1818, 1831-1843, doi:10.1016/j.bbamem.2011.08.006 (2012).
14 Chen, W. et al. Glioma cells escaped from cytotoxicity of temozolomide and vincristine by communicating with human astrocytes. Medical oncology 32, 43, doi:10.1007/s12032-015-0487-0 (2015).
15 Gielen, P. R. et al. Connexin43 confers Temozolomide resistance in human glioma cells by modulating the mitochondrial apoptosis pathway. Neuropharmacology 75, 539-548, doi:10.1016/j.neuropharm.2013.05.002 (2013).
16 Munoz, J. L. et al. Temozolomide resistance in glioblastoma cells occurs partly through epidermal growth factor receptor-mediated induction of connexin 43. Cell Death Dis 5, e1145, doi:10.1038/cddis.2014.111 (2014).
17 Lai, S. W. et al. Differential Characterization of Temozolomide-Resistant Human Glioma Cells. Int J Mol Sci 19, doi:10.3390/ijms19010127 (2018).
18 Yusubalieva, G. M. et al. Treatment of poorly differentiated glioma using a combination of monoclonal antibodies to extracellular connexin-43 fragment, temozolomide, and radiotherapy. Bull Exp Biol Med 157, 510-515, doi:10.1007/s10517-014-2603-0 (2014).
19 Zhang, X. H., Qian, Y., Li, Z., Zhang, N. N. & Xie, Y. J. Let-7g-5p inhibits epithelial-mesenchymal transition consistent with reduction of glioma stem cell phenotypes by targeting VSIG4 in glioblastoma. Oncol Rep 36, 2967-2975, doi:10.3892/or.2016.5098 (2016).
20 Wang, L. et al. Tramadol attenuates the sensitivity of glioblastoma to temozolomide through the suppression of Cx43 mediated gap junction intercellular communication. Int J Oncol 52, 295-304, doi:10.3892/ijo.2017.4188 (2018).
21 Murphy, S. F. et al. Connexin 43 Inhibition Sensitizes Chemoresistant Glioblastoma Cells to Temozolomide. Cancer Res 76, 139-149, doi:10.1158/0008-5472.CAN-15-1286 (2016).
22 Hunter, A. W., Barker, R. J., Zhu, C. & Gourdie, R. G. Zonula occludens-1 alters connexin43 gap junction size and organization by influencing channel accretion. Molecular biology of the cell 16, 5686-5698, doi:10.1091/mbc.E05-08-0737 (2005).
23 Kanabur, P. et al. Patient-derived glioblastoma stem cells respond differentially to targeted therapies. Oncotarget 7, 86406-86419, doi:10.18632/oncotarget.13415 (2016).
24 Pridham, K. J. et al. PIK3CB/p110beta is a selective survival factor for glioblastoma. Neuro Oncol 20, 494-505, doi:10.1093/neuonc/nox181 (2018).
25 Sheng, K. L., Pridham, K. J., Sheng, Z., Lamouille, S. & Varghese, R. T. Functional Blockade of Small GTPase RAN Inhibits Glioblastoma Cell Viability. Front Oncol 8, 662, doi:10.3389/fonc.2018.00662 (2018).
26 Varghese, R. T. et al. Casein Kinase 1 Epsilon Regulates Glioblastoma Cell Survival. Sci Rep 8, 13621, doi:10.1038/s41598-018-31864-x (2018).
27 Roberts, R. et al. Development of PLGA nanoparticles for sustained release of a connexin43 mimetic peptide to target glioblastoma cells. Mater Sci Eng C Mater Biol Appl 108, 110191, doi:10.1016/j.msec.2019.110191 (2020).
28 Sheng, Z., Lewis, J. A. & Chirico, W. J. Nuclear and nucleolar localization of 18-kDa fibroblast growth factor-2 is controlled by C-terminal signals. J Biol Chem 279, 40153-40160, doi:10.1074/jbc.M400123200 (2004).
29 Sheng, Z., Liang, Y., Lin, C. Y., Comai, L. & Chirico, W. J. Direct regulation of rRNA transcription by fibroblast growth factor 2. Mol Cell Biol 25, 9419-9426, doi:10.1128/MCB.25.21.9419-9426.2005 (2005).
30 Wu, S. et al. PARP mediated PARylation of MGMT is critical to promote repair of temozolomide-induced O6-methylguanine DNA damage in glioblastoma. Neuro Oncol, doi:10.1093/neuonc/noab003 (2021).
31 Liu, Q., Yin, X., Languino, L. R. & Altieri, D. C. Evaluation of drug combination effect using a Bliss independence dose-response surface model. Stat Biopharm Res 10, 112-122, doi:10.1080/19466315.2018.1437071 (2018).
32 Guo, S. et al. A rapid and high content assay that measures cyto-ID-stained autophagic compartments and estimates autophagy flux with potential clinical applications. Autophagy 11, 560-572, doi:10.1080/15548627.2015.1017181 (2015).
33 Bowman, R. L., Wang, Q., Carro, A., Verhaak, R. G. & Squatrito, M. GlioVis data portal for visualization and analysis of brain tumor expression datasets. Neuro Oncol 19, 139-141, doi:10.1093/neuonc/now247 (2017).
34 Ghandi, M. et al. Next-generation characterization of the Cancer Cell Line Encyclopedia. Nature 569, 503-508, doi:10.1038/s41586-019-1186-3 (2019).
35 Uhlen, M. et al. Proteomics. Tissue-based map of the human proteome. Science 347, 1260419, doi:10.1126/science.1260419 (2015).
36 Ek-Vitorin, J. F., King, T. J., Heyman, N. S., Lampe, P. D. & Burt, J. M. Selectivity of connexin 43 channels is regulated through protein kinase C-dependent phosphorylation. Circ Res 98, 1498-1505, doi:10.1161/01.RES.0000227572.45891.2c (2006).
37 Li, W., Hertzberg, E. L. & Spray, D. C. Regulation of connexin43-protein binding in astrocytes in response to chemical ischemia/hypoxia. J Biol Chem 280, 7941-7948, doi:10.1074/jbc.M410548200 (2005).
38 Leithe, E., Mesnil, M. & Aasen, T. The connexin 43 C-terminus: A tail of many tales. Biochim Biophys Acta, doi:10.1016/j.bbamem.2017.05.008 (2017).
39 O'Quinn, M. P., Palatinus, J. A., Harris, B. S., Hewett, K. W. & Gourdie, R. G. A peptide mimetic of the connexin43 carboxyl terminus reduces gap junction remodeling and induced arrhythmia following ventricular injury. Circ Res 108, 704-715, doi:10.1161/CIRCRESAHA.110.235747 (2011).
40 Chi, Y. et al. Purinergic control of AMPK activation by ATP released through connexin 43 hemichannels—pivotal roles in hemichannel-mediated cell injury. J Cell Sci 127, 1487-1499, doi:10.1242/jcs.139089 (2014).
41 Ujiie, H., Chaytor, A. T., Bakker, L. M. & Griffith, T. M. Essential role of Gap junctions in NO- and prostanoid-independent relaxations evoked by acetylcholine in rabbit intracerebral arteries. Stroke 34, 544-550 (2003).
42 Fiori, M. C., Reuss, L., Cuello, L. G. & Altenberg, G. A. Functional analysis and regulation of purified connexin hemichannels. Front Physiol 5, 71, doi:10.3389/fphys.2014.00071 (2014).
43 Sasaki, T. et al. Function of PI3Kgamma in thymocyte development, T cell activation, and neutrophil migration. Science 287, 1040-1046 (2000).
44 Mateo, J. et al. A first-time-in-human study of GSK2636771, a phosphoinositide 3 kinase beta-selective inhibitor, in patients with advanced solid tumors. Clin Cancer Res, doi:10.1158/1078-0432.CCR-17-0725 (2017).
45 Mulkearns-Hubert, E. E. et al. Development of a Cx46 Targeting Strategy for Cancer Stem Cells. Cell Rep 27, 1062-1072 e1065, doi:10.1016/j.celrep.2019.03.079 (2019).
46 Hitomi, M. et al. Differential connexin function enhances self-renewal in glioblastoma. Cell Rep 11, 1031-1042, doi:10.1016/j.celrep.2015.04.021 (2015).
47 Jimenez, T., Fox, W. P., Naus, C. C., Galipeau, J. & Belliveau, D. J. Connexin over-expression differentially suppresses glioma growth and contributes to the bystander effect following HSV-thymidine kinase gene therapy. Cell Commun Adhes 13, 79-92, doi:10.1080/15419060600631771 (2006).
48 Goldberg, G. S. et al. Connexin43 suppresses MFG-E8 while inducing contact growth inhibition of glioma cells. Cancer Res 60, 6018-6026 (2000).
49 Yoshimura, T., Satake, M., Ohnishi, A., Tsutsumi, Y. & Fujikura, Y. Mutations of connexin32 in Charcot-Marie-Tooth disease type X interfere with cell-to-cell communication but not cell proliferation and myelin-specific gene expression. J Neurosci Res 51, 154-161, doi:10.1002/(SICI)1097-4547(19980115)51:2<154::AID-JNR4>3.0.CO;2-C (1998).
50 Arun, S., Ravisankar, S. & Vanisree, A. J. Implication of connexin30 on the stemness of glioma: connexin30 reverses the malignant phenotype of glioma by modulating IGF-1R, CD133 and cMyc. J Neurooncol 135, 473-485, doi:10.1007/s11060-017-2608-4 (2017).
51 Arun, S., Vanisree, A. J. & Ravisankar, S. Connexin 30 downregulates Insulin-like growth factor receptor-1, abolishes Erk and potentiates effects of an IGF-R inhibitor in a glioma cell line. Brain Res 1643, 80-90, doi:10.1016/j.brainres.2016.04.061 (2016).
52 Artesi, M. et al. Connexin 30 expression inhibits growth of human malignant gliomas but protects them against radiation therapy. Neuro Oncol 17, 392-406, doi:10.1093/neuonc/nou215 (2015).
53 Cotrina, M. L., Lin, J. H. & Nedergaard, M. Adhesive properties of connexin hemichannels. Glia 56, 1791-1798, doi:10.1002/glia.20728 (2008).
54 Fu, C. T., Bechberger, J. F., Ozog, M. A., Perbal, B. & Naus, C. C. CCN3 (NOV) interacts with connexin43 in C6 glioma cells: possible mechanism of connexin-mediated growth suppression. J Biol Chem 279, 36943-36950, doi:10.1074/jbc.M403952200 (2004).
55 Naus, C. C., Elisevich, K., Zhu, D., Belliveau, D. J. & Del Maestro, R. F. In vivo growth of C6 glioma cells transfected with connexin43 cDNA. Cancer research 52, 4208-4213 (1992).
56 Soroceanu, L., Manning, T. J., Jr. & Sontheimer, H. Reduced expression of connexin-43 and functional gap junction coupling in human gliomas. Glia 33, 107-117 (2001).
57 Aasen, T., Mesnil, M., Naus, C. C., Lampe, P. D. & Laird, D. W. Gap junctions and cancer: communicating for 50 years. Nat Rev Cancer 16, 775-788, doi:10.1038/nrc.2016.105 (2016).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An engineered peptide comprising:
   a p110beta targeting peptide; and
   a delivery moiety, wherein the delivery moiety is coupled to the p110beta targeting peptide.
2. The engineered peptide of aspect 1, wherein the p110beta targeting peptide is capable of selectively binding p110beta or a complex thereof, selectively inhibiting p110beta activity, or both.
3. The engineered peptide of any one of aspects 1-2, wherein the p110beta targeting peptide comprises an amino acid sequence that is about 95 to 100 identical to SEQ ID NO: 3 or is a homologue thereof, or functional variant thereof.
4. The engineered peptide of any one of aspects 1-3, wherein the delivery moiety is a cell penetrating peptide.
5. The engineered peptide of any one of aspects 1-4, wherein the cell penetrating peptide has a sequence identical to SEQ ID NO: 4, SEQ ID NO: 6, or any one of SEQ ID NOs: 110-222.
6. The engineered peptide any one of aspects 1-5, wherein one or more amino acids of the engineered peptide comprises one or more ester linked groups.
7. A pharmaceutical formulation comprising:
   an engineered peptide of any one of any one of aspects 1-6; and
   a pharmaceutically acceptable carrier.
8. The pharmaceutical formulation of aspect 7, further comprising:
   a. a connexin 43 inhibitor;
   b. a chemotherapeutic;
   c. an immune checkpoint inhibitor; or
   d. any combination thereof.
9. The pharmaceutical formulation of aspect 8, wherein the Cx43 inhibitor is a biologic molecule-based inhibitor, a chemical molecule inhibitor, a small molecule inhibitor, an RNAi-based inhibitor, or a genetic modifier-based inhibitor.
10. The pharmaceutical formulation any one of aspects 8-9, wherein the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.
11. The pharmaceutical formulation of any one of aspects 8-10, wherein the chemotherapeutic is not effective to treat a cancer or a chemotherapeutic resistant cancer having overexpression of p110beta when used alone.
12. The pharmaceutical formulation of any one of aspects 8-11, wherein the chemotherapeutic is temozolomide.
13. An exosome comprising:
   an engineered peptide of any one of aspects 1-6.
14. The exosome of aspect 13, wherein the exosome further comprises
   a. a connexin 43 inhibitor;
   b. a chemotherapeutic;
   c. an immune checkpoint inhibitor; or
   d. any combination thereof.
15. The exosome of aspect 14, wherein the Cx43 inhibitor is a biologic molecule-based inhibitor, a chemical molecule inhibitor, a small molecule inhibitor, an RNAi-based inhibitor, or a genetic modifier-based inhibitor.
16. The exosome of any one of aspects 14-15, wherein the connexin 43 inhibitor is a peptide selected from the group consisting of: aCT1 (SEQ ID NO: 7), aCT1 1 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.
17. The exosome of any one of aspects 14-16, wherein the chemotherapeutic is not effective to treat a cancer or a chemotherapeutic resistant cancer having overexpression of p110beta when used alone.
18. The exosome of any one of aspects 14-17, wherein the chemotherapeutic is temozolomide.
19. The exosome of any one of aspects 13-18, wherein the exosome is a milk exosome.
20. A method of treating a PI3K mediated disease or a symptom thereof in a subject in need thereof, the method comprising:
   administering, to the subject in need thereof, an engineered peptide as in any one of aspects 1-6.
21. The method of aspect 20, wherein the PI3K mediated disease is a cancer, optionally a chemotherapy resistant cancer.
22. The method of any aspect 21, wherein the cancer is characterized at least in part by overexpression of p110beta.
23. The method of any one of aspects 21-22, wherein the cancer is glioblastoma or melanoma.
24. A method of treating a PI3K mediated disease or a symptom thereof in a subject in need thereof, the method comprising:
   administering, to the subject in need thereof, a pharmaceutical formulation comprising
   an engineered peptide as in any one of aspects 1-6; and
   a pharmaceutically acceptable carrier.
25. The method of aspect 24, wherein the pharmaceutical formulation further comprises
   a. a connexin 43 inhibitor;
   b. a chemotherapeutic;
   c. an immune checkpoint inhibitor; or
   d. any combination thereof.
26. The method of aspect 25, wherein the Cx43 inhibitor is a biologic molecule-based inhibitor, a chemical molecule inhibitor, a small molecule inhibitor, an RNAi-based inhibitor, or a genetic modifier-based inhibitor.
27. The method of any one of aspects 25-26, wherein the connexin 43 inhibitor is a peptide selected from the group consisting of: aCT1 (SEQ ID NO: 7), aCT1 1 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.
28. The method of any one of aspects 25-27, wherein the chemotherapeutic is a chemotherapeutic used to treat a cancer having overexpression of p110beta.
29. The method of any one of aspects 25-28, wherein the chemotherapeutic is temozolomide.
30. The method any one of aspects 24-29, wherein the PI3K mediated disease is a cancer, optionally a chemotherapy resistant cancer.
31. The method of aspect 30, wherein the cancer is characterized at least in part by overexpression of p110beta.
32. The method of aspect 31, wherein the cancer is glioblastoma or melanoma.
33. A method of treating a PI3K mediated disease or a symptom thereof in a subject in need thereof, the method comprising:
   administering, to the subject in need thereof, an exosome of aspect 13 or a pharmaceutical formulation thereof.
34. The method of aspect 33, wherein the exosome further comprises
   a. a connexin 43 inhibitor;
   b. a chemotherapeutic;
   c. an immune checkpoint inhibitor; or
   d. any combination thereof.
35. The method of aspect 34, wherein the Cx43 inhibitor is a biologic molecule-based inhibitor, a chemical molecule inhibitor, a small molecule inhibitor, an RNAi-based inhibitor, or a genetic modifier-based inhibitor.
36. The method of any one of aspects 34-35, wherein the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.
37. The method any one of aspects 34-36, wherein the chemotherapeutic is not effective to treat a cancer or a chemotherapeutic resistant cancer having overexpression of p110beta when used alone.
38. The method of any one of aspects 34-37, wherein the chemotherapeutic is temozolomide.
39. The method of any one of aspects 33-38, wherein the PI3K mediated disease is a cancer, optionally a chemotherapy resistant cancer.
40. The method of aspect 39, wherein the cancer is characterized at least in part by overexpression of p110beta.
41. The method of any one of aspects 39-40, wherein the cancer is glioblastoma or melanoma.
42. A kit comprising the engineered peptide of any one of aspects 1-6, a pharmaceutical formulation as in any of aspects 7-12, an exosome as in any one of aspects 13-19, or any combination thereof.
43. A pharmaceutical formulation comprising:
   a. a connexin 43 inhibitor;
   b. a chemotherapeutic; and optionally
   c. a PI3K inhibitor; and
   a pharmaceutically acceptable carrier.
44. The pharmaceutical formulation of aspect 43, wherein the Cx43 inhibitor is a biologic molecule-based inhibitor, a chemical molecule inhibitor, a small molecule inhibitor, an RNAi-based inhibitor, or a genetic modifier-based inhibitor.
45. The pharmaceutical formulation of any one of aspects 43-44, wherein the connexin 43 inhibitor is a peptide selected from the group consisting of: aCT1 (SEQ ID NO: 7), aCT1 1 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.
46. The pharmaceutical formulation of any one of aspects 43-45, wherein the PI3K inhibitor is a selective p110beta inhibitor.
47. The pharmaceutical formulation of aspect 46, wherein the selective p110beta inhibitor is a biologic molecule-based inhibitor, a chemical molecule inhibitor, a small molecule inhibitor, an RNAi-based inhibitor, or a genetic modifier-based inhibitor.
48. The pharmaceutical formulation of any one of aspects 46-47, wherein the selective p110beta inhibitor is an engineered peptide comprising a p110beta targeting peptide; and a delivery moiety, wherein the delivery moiety is coupled to the p110beta targeting peptide, wherein the engineered peptide optionally comprises one or more ester-linked groups.
49. The pharmaceutical formulation of aspect 48, wherein the p110beta targeting peptide is capable of selectively binding p110beta or a complex thereof, selectively inhibiting p110beta activity, or both.
50. The pharmaceutical formulation of any one of aspects 48-49, wherein the p110beta targeting peptide comprises an amino acid sequence that is about 95 to 100 identical to SEQ ID NO: 3 or is a homologue thereof, or functional variant thereof.
51. The pharmaceutical formulation of any one of aspects 48-50, wherein the delivery moiety is a cell penetrating peptide.
52. The pharmaceutical formulation of aspect 51, wherein the cell penetrating peptide has a sequence identical to SEQ ID NO: 4, SEQ ID NO: 6, or any one of SEQ ID NOs. 110-222.
53. The pharmaceutical formulation of any one of aspects 43-52, wherein the PI3K inhibitor is a biologic molecule-based inhibitor, a chemical molecule inhibitor, a small molecule inhibitor, an RNAi-based inhibitor, or a genetic modifier-based inhibitor.
54. The pharmaceutical formulation of any one of aspects 43-53, wherein the chemotherapeutic is not effective to treat a cancer or a chemotherapeutic resistant cancer having overexpression of p110beta, Cx43, or both when used alone.
55. The pharmaceutical formulation of any one of aspects 43-54, wherein the chemotherapeutic is temozolomide.
56. An exosome comprising:
   a. a connexin 43 inhibitor;
   b. a chemotherapeutic; and optionally
   c. a PI3K inhibitor.
57. The exosome of aspect 56, wherein the Cx43 inhibitor is a biologic molecule-based inhibitor, a chemical molecule inhibitor, a small molecule inhibitor, an RNAi-based inhibitor, or a genetic modifier-based inhibitor.
58. The exosome of any one of aspects 56-57, wherein the connexin 43 inhibitor is a peptide selected from the group consisting of: aCT1 (SEQ ID NO: 7), aCT1 1 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.
59. The exosome of any one of aspects 56-57, wherein the PI3K inhibitor is a selective p110beta inhibitor.
60. The exosome of aspect 59, wherein the selective p110beta inhibitor is a biologic molecule-based inhibitor, a chemical molecule inhibitor, a small molecule inhibitor, an RNAi-based inhibitor, or a genetic modifier-based inhibitor.
61. The exosome of any one of aspects 59-60, wherein the selective p110beta inhibitor is an engineered peptide comprising a p110beta targeting peptide; and a delivery moiety, wherein the delivery moiety is coupled to the p110beta targeting peptide, wherein the engineered peptide optionally comprises one or more ester-linked groups.
62. The exosome of any one of aspects 59-61, wherein the p110beta targeting peptide is capable of selectively binding p110beta or a complex thereof, selectively inhibiting p110beta activity, or both.
63. The exosome of any one of aspects 61-62, wherein the p110beta targeting peptide comprises an amino acid sequence that is about 95 to 100 identical to SEQ ID NO: 3 or is a homologue thereof, or functional variant thereof.
64. The exosome of aspect 61-63, wherein the delivery moiety is a cell penetrating peptide.
65. The exosome of aspect 64, wherein the cell penetrating peptide has a sequence identical to SEQ ID NO: 4, SEQ ID NO: 6, or any one of SEQ ID NOs: 110-222.
66. The exosome of any one of aspects 56-65, wherein the PI3K inhibitor is a biologic molecule-based inhibitor, a chemical molecule inhibitor, a small molecule inhibitor, an RNAi-based inhibitor, or a genetic modifier-based inhibitor.
67. The exosome of any one of aspects 56-66, wherein the chemotherapeutic is not effective to treat a cancer or a chemotherapeutic resistant cancer having overexpression of p110beta, Cx43, or both when used alone.
68. The exosome of any one of aspects 56-67, wherein the chemotherapeutic is temozolomide.
69. A method of treating a PI3K mediated disease or a symptom thereof in a subject in need thereof, the method comprising:
   administering, to the subject in need thereof, a pharmaceutical formulation as in any one of aspects 43-55 or an exosome as in any one of aspects 56-68, or an amount of a connexin 43 inhibitor, an amount of a chemotherapeutic, and an amount of a PI3K inhibitor.
70. The method of aspects 69, wherein the Cx43 inhibitor is a biologic molecule-based inhibitor, a chemical molecule inhibitor, a small molecule inhibitor, an RNAi-based inhibitor, or a genetic modifier-based inhibitor.
71. The method of aspect 70, wherein the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof, wherein the peptide optionally comprises one or more ester-linked groups.
72. The method of any one of aspects 69-71, wherein the PI3K inhibitor is a selective p110beta inhibitor.
73. The method of aspect 72, wherein the selective p110beta inhibitor is a biologic molecule-based inhibitor, a chemical molecule inhibitor, a small molecule inhibitor, an RNAi-based inhibitor, or a genetic modifier-based inhibitor.
74. The method of any one of aspects 72-73, wherein the selective p110beta inhibitor is an engineered peptide comprising a p110beta targeting peptide; and a delivery moiety, wherein the delivery moiety peptide is coupled to the p110beta targeting peptide, wherein the engineered peptide optionally comprises one or more ester-linked groups.
75. The method of aspect 74, wherein the p110beta targeting peptide comprises an amino acid sequence that is about 95 to 100 identical to SEQ ID NO: 3 or is a homologue thereof, or functional variant thereof.
76. The method of any one of aspects 74-75, wherein the delivery moiety is a cell penetrating peptide.
77. The method of aspect 76, wherein the cell penetrating peptide has a sequence identical to SEQ ID NO: 4, SEQ ID NO: 6, or any one of SEQ ID NOs: 110-222.
78. The method of any one of aspects 69-77, wherein the PI3K inhibitor is a biologic molecule-based inhibitor, a chemical molecule inhibitor, a small molecule inhibitor, an RNAi-based inhibitor, or a genetic modifier-based inhibitor.
79. The method of any one of aspects 69-78, wherein the chemotherapeutic is not effective to treat a cancer or a chemotherapeutic resistant cancer having overexpression of p110beta, Cx43, or both when used alone.
80. The method of any one of aspects 69-79, wherein the chemotherapeutic is temozolomide.
81. The method of any one of aspects 69-80, wherein the PI3K mediated disease is a cancer, optionally a chemotherapy resistant cancer.
82. The method of any one of aspects 69-82, wherein the cancer is characterized at least in part by overexpression of p110beta, Cx43, or both.
83. The method of any one of aspects 69-83, wherein the cancer is glioblastoma or melanoma.
84. A kit comprising: the pharmaceutical formulation of any one of aspects 43-55, an exosome of any one of aspects 56-68, or an amount of a connexin 43 inhibitor, an amount of a chemotherapeutic, and an amount of a PI3K inhibitor.
85. A polynucleotide encoding an engineered peptide of any one of aspects 1-6.
86. A vector, optionally an expression vector, comprising a polynucleotide of aspect 85.
87. A cell or cell population comprising a polynucleotide of aspect 85, a vector of claim 86, or both.

1. An engineered peptide comprising:
a p110beta targeting peptide; and
a delivery moiety, wherein the delivery moiety is coupled to the p110beta targeting peptide.

2. The engineered peptide of claim 1, wherein the p110beta targeting peptide is capable of selectively binding p110beta or a complex thereof, selectively inhibiting p110beta activity, or both.

3. The engineered peptide of claim 1, wherein the p110beta targeting peptide comprises an amino acid sequence that is about 95 to 100 identical to SEQ ID NO: 3 or is a homologue thereof, or functional variant thereof.

4. The engineered peptide of claim 1, wherein the delivery moiety is a cell penetrating peptide.

5. The engineered peptide of claim 4, wherein the cell penetrating peptide has a sequence identical to SEQ ID NO: 4, SEQ ID NO: 6, or any one of SEQ ID NOs: 110-222.

6. The engineered peptide of claim 1, wherein one or more amino acids of the engineered peptide comprises one or more ester linked groups.

7.-87. (canceled)

88. A pharmaceutical formulation or an exosome comprising:
(a) an engineered peptide of claim 1;
(b) a connexin 43 inhibitor;
(c) a chemotherapeutic;
(d) an immune checkpoint inhibitor;
(e) a PI3K inhibitor;
(f) a combination of (b) and (c);
(g) a combination of (b), (c), and (e); or
(h) any combination of (a)-(e),
wherein the pharmaceutical formulation further comprises a pharmaceutically acceptable carrier.

89. The pharmaceutical formulation or exosome of claim 88, wherein
(a) the connexin 43 inhibitor is a biologic molecule-based inhibitor, a chemical molecule inhibitor, a small molecule inhibitor, an RNAi-based inhibitor, or a genetic modifier-based inhibitor;
(b) the chemotherapeutic is not effective to treat a cancer or a chemotherapeutic resistant cancer having overexpression of p110beta when used alone;
(c) wherein the PI3K inhibitor is a biologic molecule-based inhibitor, a chemical molecule inhibitor, a small molecule inhibitor, an RNAi-based inhibitor, or a genetic modifier-based inhibitor; or
(d) any combination of (a)-(c).

90. The pharmaceutical formulation or exosome of claim 89, wherein
(a) the connexin 43 inhibitor is a peptide selected from the group consisting of: aCT1 (SEQ ID NO: 7), aCT11 (SEQ ID NO: 8), aCT1 minus I (SEQ ID NO: 9), aCT11 minus I (SEQ ID NO: 10), a peptide comprising an amino acid sequence identical to any one of SEQ ID NOs: 11-109, or any combination thereof;
(b) the chemotherapeutic is temozolomide;
(c) the PI3K inhibitor is a selective p110beta inhibitor; or
(d) any combination of (a)-c).

91. The pharmaceutical formulation or exosome of claim 90, wherein the connexin 43 inhibitor comprises one or more ester-linked groups.

92. The pharmaceutical formulation or exosome of claim 91, wherein the selective p110beta inhibitor comprises an engineered peptide comprising a p110beta targeting peptide; and a delivery moiety, wherein the delivery moiety is coupled to the p110beta targeting peptide.

93. The pharmaceutical formulation or exosome of claim 93, (a) wherein the delivery moiety is a cell penetrating peptide; (b) wherein the p110beta targeting peptide is capable of selectively binding p110beta or a complex thereof, selectively inhibiting p110beta activity, or both; or (c) both (a) and (b).

94. The pharmaceutical formulation or exosome of claim 93, wherein (a) the cell penetrating peptide has a sequence identical to SEQ ID NO: 4, SEQ ID NO: 6, or any one of SEQ ID NOs: 110-222; (b) wherein the p110beta targeting peptide comprises an amino acid sequence that is about 95 to 100 identical to SEQ ID NO: 3 or is a homologue thereof, or functional variant thereof; or (c) both (a) and (b).

95. The pharmaceutical formulation or exosome of claim 90, wherein (a) the engineered peptide comprises one or more ester-linked groups, (b) the exosome is a milk exosome, or (c) both (a) and (b).

96. A polynucleotide encoding an engineered a peptide of claim 1, a vector comprising the polynucleotide, or a cell or cell population comprising the engineered peptide, the vector, or both.

97. A kit comprising:
(a) an engineered polypeptide of claim 1;
(b) a pharmaceutical formulation or exosome comprising:
(i) an engineered peptide of claim 1;
(ii) a connexin 43 inhibitor;
(iii) a chemotherapeutic;
(iv) an immune checkpoint inhibitor;
(v) a PI3K inhibitor;
(vi) a combination of (ii) and (iii);
(vii) a combination of (ii), (iii), and (v); or
(viii) any combination of (i)-(vi),
wherein the pharmaceutical formulation further comprises a pharmaceutically acceptable carrier;
(c) polynucleotide encoding an engineered a peptide of claim 1;
(d) a vector comprising the polynucleotide of (c);
(e) a cell or cell population any combination of (a)-(d).

98. A method of treating a PI3K meditated disease or a symptom thereof in a subject in need thereof, the method comprising:
administering, to the subject in need thereof
(a) an engineered polypeptide of claim 1;
(b) a pharmaceutical formulation or exosome comprising:
(i) an engineered peptide of claim 1;
(ii) a connexin 43 inhibitor;
(iii) a chemotherapeutic;
(iv) an immune checkpoint inhibitor;
(v) a PI3K inhibitor;
(vi) a combination of (ii) and (iii);
(vii) a combination of (ii), (iii), and (v); or
(viii) any combination of (i)-(vi),
wherein the pharmaceutical formulation further comprises a pharmaceutically acceptable carrier;
(c) polynucleotide encoding an engineered a peptide of claim 1;
(d) a vector comprising the polynucleotide of (c);
(e) a cell or cell population any combination of (a)-(d); or
(f) any combination of (a)-(e).

99. The method of claim 98, wherein the PI3K mediated disease is a cancer.

100. The method of claim 99, wherein (a) the cancer is a chemotherapy resistant cancer, (b) wherein the cancer is characterized at least in part by overexpression of p110beta, or (c) both (a) and (b).

101. The method of claim 99, wherein the cancer is a glioblastoma or a melanoma.


SEQUENCE LISTING (222 sequences)

SEQ ID NO: 1 - protein, 26 aa, Artificial Sequence
  p110bC2in - Selectide-18 with a cell penetrating peptide
RRRRRRRRTKKSTKTINPSKYQTIRK

SEQ ID NO: 2 - protein, 34 aa, Artificial Sequence
  Ante-Selectide-18, Selectide-18 with antennapedia cell penetrating peptide
RQPKIWFPNRRKPWKKTKKSTKTINPSKYQTIRK

SEQ ID NO: 3 - protein, 18 aa, Artificial Sequence
  Selectide-18
TKKSTKTINPSKYQTIRK

SEQ ID NO: 4 - protein, 8 aa, Artificial Sequence
  Cell penetrating peptide
RRRRRRRR

SEQ ID NO: 5 - protein, 18 aa, Artificial Sequence
  Scrambled control peptide
IYKTSKTRSQKTKIKPNT

SEQ ID NO: 6 - protein, 16 aa, Artificial Sequence
  Antennapedia cell penetrating sequence
RQPKIWFPNRRKPWKK

SEQ ID NO: 7 - protein, 25 aa, Artificial Sequence
  aCT1 peptide
RQPKIWFPNRRKPWKKRPRPDDLEI

SEQ ID NO: 8 - protein, 24 aa, Artificial Sequence
  aCT11 peptide
RQPKIWFPNRRKPWKKRPRPDDLE

SEQ ID NO: 9 - protein, 9 aa, Artificial Sequence
  aCT1 minus peptide
RPRPDDLEI

SEQ ID NO: 10 - protein, 8 aa, Artificial Sequence
  aCT11 minus I (aCT11-I) peptide
RPRPDDLE

SEQ ID NO: 11 - protein, 6 aa, Artificial Sequence
  aCT variant peptide
RPRPDD

SEQ ID NO: 12 - protein, 10 aa, Artificial Sequence
  aCT variant peptide
SRPRPDDLEI

SEQ ID NO: 13 - protein, 9 aa, Artificial Sequence
  aCT variant peptide
SRPRPDDLE

SEQ ID NO: 14 - protein, 7 aa, Artificial Sequence
  aCT variant peptide
SRPRPDD

SEQ ID NO: 15 - protein, 22 aa, Artificial Sequence
  aCT variant peptide
IVDQRPSSRASSRASSRPRPDD

SEQ ID NO: 16 - protein, 20 aa, Artificial Sequence
  aCT variant peptide
PSSRASSRASSRPRPDDLEI

SEQ ID NO: 17 - protein, 9 aa, Artificial Sequence
  aCT variant peptide
RARPDDLDV

SEQ ID NO: 18 - protein, 9 aa, Artificial Sequence
  p110bC2in - Selectide-18 with a cell penetrating peptide
GDGKNSWWI

SEQ ID NO: 19 - protein, 10 aa, Artificial Sequence
  aCT variant peptide
GRARPEDLAI

SEQ ID NO: 20 - protein, 8 aa, Artificial Sequence
  aCT variant peptide
RDGKTVWI

SEQ ID NO: 21 - protein, 11 aa, Artificial Sequence
  aCT variant peptide
GRTQSSDSAYW

SEQ ID NO: 22 - protein, 12 aa, Artificial Sequence
  aCT variant peptide
KASSKARSDDSW

SEQ ID NO: 23 - protein, 9 aa, Artificial Sequence
  aCT variant peptide
CSGKSKKPW

SEQ ID NO: 24 - protein, 22 aa, Artificial Sequence
  aCT variant peptide
IVDQRPSSRASSRASSRPRPDD

SEQ ID NO: 25 - protein, 20 aa, Artificial Sequence
  aCT variant peptide
PSSRASSRASSRPRPDDLEI

SEQ ID NO: 26 - protein, 5 aa, Artificial Sequence
  aCT variant peptide
DDLEI

SEQ ID NO: 27 - protein, 4 aa, Artificial Sequence
  aCT variant peptide
DLEI

SEQ ID NO: 28 - skipped

SEQ ID NO: 29 - protein, 8 aa, Artificial Sequence
  aCT variant peptide
PRPDDLEI

SEQ ID NO: 30 - protein, 7 aa, Artificial Sequence
  aCT variant peptide
RPDDLEI

SEQ ID NO: 31 - protein, 6 aa, Artificial Sequence
  aCT variant peptide
PDDLEI

SEQ ID NO: 32 - protein, 6 aa, Artificial Sequence
  aCT variant peptide
RPDDLE

SEQ ID NO: 33 - protein, 9 aa, Artificial Sequence
  aCT variant peptide
RPRPDDELI

SEQ ID NO: 34 - protein, 9 aa, Artificial Sequence
  aCT variant peptide
KPRPDDLEI

SEQ ID NO: 35 - protein, 9 aa, Artificial Sequence
  aCT variant peptide
RPRPDDLEV

SEQ ID NO: 36 - protein, 9 aa, Artificial Sequence
  aCT variant peptide
RPRPDDVPV

SEQ ID NO: 37 - protein, 9 aa, Artificial Sequence
  aCT variant peptide
RPKPDDLEI

SEQ ID NO: 38 - protein, 19 aa, Artificial Sequence
  aCT variant peptide
SSRASSRASSRPKPDDLEI

SEQ ID NO: 39 - protein, 6 aa, Artificial Sequence
  aCT variant peptide
RPKPDD

SEQ ID NO: 40 - protein, 19 aa, Artificial Sequence
  aCT variant peptide
SSRASSRASSRPRPDDLDI

SEQ ID NO: 41 - protein, 19 aa, Artificial Sequence
  aCT variant peptide
SSRASTRASSRPRPDDLEI

SEQ ID NO: 42 - protein, 9 aa, Artificial Sequence
  aCT variant peptide
RPRPEDLEI

SEQ ID NO: 43 - protein, 19 aa, Artificial Sequence
  aCT variant peptide
SSRASSRASSRPRPEDLEI

SEQ ID NO: 44 - protein, 9 aa, Artificial Sequence
  aCT variant peptide
GDGKNSVWV

SEQ ID NO: 45 - protein, 23 aa, Artificial Sequence
  aCT variant peptide
SKAGSNKSTASSKSGDGKNSVWV

SEQ ID NO: 46 - protein, 18 aa, Artificial Sequence
  aCT variant peptide
GQKPPSRPSSSASKKLYV

SEQ ID NO: 47 - protein, 10 aa, Artificial Sequence
  aCT variant peptide
DRPRPDDLEI

SEQ ID NO: 48 - protein, 11 aa, Artificial Sequence
  aCT variant peptide
EERPRPDDLEI

SEQ ID NO: 49 - protein, 9 aa, Artificial Sequence
  aCT variant peptide
ERPRPDDEL

SEQ ID NO: 50 - protein, 11 aa, Artificial Sequence
  aCT variant peptide
DDRPRPDDELI

SEQ ID NO: 51 - protein, 15 aa, Artificial Sequence
  Cx43 JM peptide
VFFKGVKDRVKGKSD

SEQ ID NO: 52 - protein, 10 aa, Artificial Sequence
  Cx43 JM peptide
VFFKGVKDRV

SEQ ID NO: 53 - protein, 20 aa, Artificial Sequence
  Cx43 JM peptide
VFFKGVKDRVKGRSDPYHAT

SEQ ID NO: 54 - protein, 9 aa, Artificial Sequence
  Cx43 JM peptide
FFKGVKDRV

SEQ ID NO: 55 - protein, 8 aa, Artificial Sequence
  Cx43 JM peptide
FKGVKDRV

SEQ ID NO: 56 - protein, 9 aa, Artificial Sequence
  Cx43 JM peptide
VFFKGVKDR

SEQ ID NO: 57 - protein, 8 aa, Artificial Sequence
  Cx43 JM peptide
VFFKGVKD

SEQ ID NO: 58 - protein, 13 aa, Artificial Sequence
  Cx43 JM peptide
DRVKGRSDPYHAT

SEQ ID NO: 59 - protein, 11 aa, Artificial Sequence
  Cx43 JM peptide
VKGRSDPYHAT

SEQ ID NO: 60 - protein, 15 aa, Artificial Sequence
  Cx43 JM peptide
VFFKGVKDRVKGQSD

SEQ ID NO: 61 - protein, 15 aa, Artificial Sequence
  Cx43 JM peptide
VFFKGIKDRVKGRND

SEQ ID NO: 62 - protein, 15 aa, Artificial Sequence
  Cx43 JM peptide
VFFKGVKDRVKGRID

SEQ ID NO: 63 - protein, 15 aa, Artificial Sequence
  Cx43 JM peptide
VFFKGIKDRVKGKSD

SEQ ID NO: 64 - protein, 14 aa, Artificial Sequence
  Cx43 JM peptide
FFKGVKDRVKGKSD

SEQ ID NO: 65 - protein, 13 aa, Artificial Sequence
  Cx43 JM peptide
FKSVKDRIKGRSD

SEQ ID NO: 66 - protein, 15 aa, Artificial Sequence
  Cx43 JM peptide
VFFRSVKDHVKGKSD

SEQ ID NO: 67 - protein, 12 aa, Artificial Sequence
  Cx43 JM peptide
VFFKRIKDRVKG

SEQ ID NO: 68 - protein, 13 aa, Artificial Sequence
  Cx43 JM peptide
VLFKQIKDRVKGR

SEQ ID NO: 69 - protein, 13 aa, Artificial Sequence
  Cx43 JM peptide
VLFKRIKDRVKGR

SEQ ID NO: 70 - protein, 15 aa, Artificial Sequence
  Cx43 JM peptide
VFFKGVKDRVKGKSD

SEQ ID NO: 71 - protein, 10 aa, Artificial Sequence
  Cx43 JM peptide
VFFKGVKDRV

SEQ ID NO: 72 - protein, 15 aa, Artificial Sequence
  Cx43 JM peptide
IFFKGVKDRVKGKSD

SEQ ID NO: 73 - protein, 10 aa, Artificial Sequence
  Cx43 JM peptide
IFFKGVKDRV

SEQ ID NO: 74 - protein, 15 aa, Artificial Sequence
  Cx43 JM peptide
VIFKRMKDQIRESEK

SEQ ID NO: 75 - protein, 15 aa, Artificial Sequence
  Cx43 JM peptide
VFFKGVKDRVKGKTD

SEQ ID NO: 76 - protein, 15 aa, Artificial Sequence
  Cx43 JM peptide
VFFKGVKDRVKGRSD

SEQ ID NO: 77 - protein, 15 aa, Artificial Sequence
  Cx43 JM peptide
VFFKGVKDRVRGKSD

SEQ ID NO: 78 - protein, 15 aa, Artificial Sequence
  Cx43 JM peptide
VFFKGVKDKVKGKSD

SEQ ID NO: 79 - protein, 15 aa, Artificial Sequence
  Cx43 JM peptide
IIFRGVRDRVRGRSD

SEQ ID NO: 80 - protein, 15 aa, Artificial Sequence
  Cx43 JM peptide
VIFKRMKDQIRESEK

SEQ ID NO: 81 - protein, 15 aa, Artificial Sequence
  Cx43 JM peptide
VIFKRMKDQIREREK

SEQ ID NO: 82 - protein, 15 aa, Artificial Sequence
  Cx43 JM peptide
VIFKRMKDKIREREK

SEQ ID NO: 83 - protein, 15 aa, Artificial Sequence
  Cx43 JM peptide
VFFKRVKDRIRERSK

SEQ ID NO: 84 - protein, 15 aa, Artificial Sequence
  Cx43 JM peptide
VFFKGVKDRVKGRSD

SEQ ID NO: 85 - protein, 39 aa, Artificial Sequence
  Cx43 JM/SRC peptide
DPYHATSGALSPAKDCGSQKYAYFNGCSSPTAPLSPMSP

SEQ ID NO: 86 - protein, 18 aa, Artificial Sequence
  Cx43 JM/SRC peptide
AYFNGCSSPTAPLSPMSP

SEQ ID NO: 87 - protein, 10 aa, Artificial Sequence
  Cx43 JM/SRC peptide
PTAPLSPMSP

SEQ ID NO: 88 - protein, 8 aa, Artificial Sequence
  Cx43 JM/SRC peptide
PTAPLSPM

SEQ ID NO: 89 - protein, 8 aa, Artificial Sequence
  Cx43 JM/SRC peptide
APLSPMSP

SEQ ID NO: 90 - protein, 30 aa, Artificial Sequence
  Cx43 H2 peptide
HAQPFDFPDDNQNSKKLAAGHELQPLAIVD

SEQ ID NO: 91 - protein, 10 aa, Artificial Sequence
  Cx43 H2 peptide
NQNSKKLAAG

SEQ ID NO: 92 - protein, 8 aa, Artificial Sequence
  Cx43 H2 peptide
NSKKLAAG

SEQ ID NO: 93 - protein, 10 aa, Artificial Sequence
  Cx43 H2 peptide
HELQPLAIVD

SEQ ID NO: 94 - protein, 9 aa, Artificial Sequence
  Cx43 C-Loop peptide
KQIEIKKFK

SEQ ID NO: 95 - protein, 26 aa, Artificial Sequence
  Cx43 C-Loop peptide
DGANVDMHLKQIEIKKFKYGIEEHGK

SEQ ID NO: 96 - protein, 11 aa, Artificial Sequence
  Cx43 C-Loop peptide
KQIEIKKFKYG

SEQ ID NO: 97 - protein, 12 aa, Artificial Sequence
  Cx43 C-Loop peptide
VDCFLSRPTEKT

SEQ ID NO: 98 - protein, 11 aa, Artificial Sequence
  Cx43 C-Loop peptide
SRPTEKTIFII

SEQ ID NO: 99 - protein, 11 aa, Artificial Sequence
  Cx43 C-Loop peptide
SRPTEKTIFLL

SEQ ID NO: 100 - protein, 7 aa, Artificial Sequence
  Cx43 C-Loop peptide
SRPTEKT

SEQ ID NO: 101 - protein, 8 aa, Artificial Sequence
  Cx43 C-Loop peptide
ESRPTEKT

SEQ ID NO: 102 - protein, 12 aa, Artificial Sequence
  Cx43 C-Loop peptide
ADCFLSRPTEKT

SEQ ID NO: 103 - protein, 12 aa, Artificial Sequence
  Cx43 C-Loop peptide
VACFLSRPTEKT

SEQ ID NO: 104 - protein, 12 aa, Artificial Sequence
  Cx43 C-Loop peptide
VDCFLSRPTAKT

SEQ ID NO: 105 - protein, 12 aa, Artificial Sequence
  Cx43 C-Loop peptide
VDCFLSRPTEAT

SEQ ID NO: 106 - protein, 10 aa, Artificial Sequence
  Cx43 C-Loop peptide
CFLSRPTEKT

SEQ ID NO: 107 - protein, 8 aa, Artificial Sequence
  Cx43 C-Loop peptide
LSRPTEKT

SEQ ID NO: 108 - protein, 9 aa, Artificial Sequence
  M3 peptide
RPRPDDLAI

SEQ ID NO: 109 - protein, 25 aa, Artificial Sequence
  M3 peptide with N-terminal antennapedia cell penetrating sequence
RQPKIWFPNRRKPWKKRPRPDDLAI

SEQ ID NO: 110 - protein, 7 aa, Artificial Sequence
  Cell penetrating peptide
PKKKRKV

SEQ ID NO: 111 - protein, 16 aa, Artificial Sequence
  Cell penetrating peptide
KRPAATKKAGQAKKKL

SEQ ID NO: 112 - protein, 8 aa, Artificial Sequence
  Cell penetrating peptide
GRKRKKRT

SEQ ID NO: 113 - protein, 8 aa, Artificial Sequence
  Cell penetrating peptide
ERKKRRRE

SEQ ID NO: 114 - protein, 7 aa, Artificial Sequence
  Cell penetrating peptide
FKKFRKF

SEQ ID NO: 115 - protein, 8 aa, Artificial Sequence
  Cell penetrating peptide
SKKKKTKV

SEQ ID NO: 116 - protein, 10 aa, Artificial Sequence
  Cell penetrating peptide
VQRKRQKLMP

SEQ ID NO: 117 - protein, 9 aa, Artificial Sequence
  Cell penetrating peptide
KLAKLAKLA

SEQ ID NO: 118 - protein, 6 aa, Artificial Sequence
  Cell penetrating peptide
PFVYLI

SEQ ID NO: 119 - protein, 27 aa, Artificial Sequence
  Cell penetrating peptide
GLAFLGFLGAAGSTNGAWSQPKKKRKV

SEQ ID NO: 120 - protein, 20 aa, Artificial Sequence
  Cell penetrating peptide
KETWWETWWTEWSQPKKRKV

SEQ ID NO: 121 - protein, 17 aa, Artificial Sequence
  Cell penetrating peptide
GALFLGFLGAAGSTMGA

SEQ ID NO: 122 - protein, 12 aa, Artificial Sequence
  Cell penetrating peptide
KETWWETWWTEW

SEQ ID NO: 123 - protein, 21 aa, Artificial Sequence
  Cell penetrating peptide
AGYLLGKINLKALAALAKKIL

SEQ ID NO: 124 - protein, 18 aa, Artificial Sequence
  Cell penetrating peptide
KLALKLALKALKAALKLA

SEQ ID NO: 125 - protein, 18 aa, Artificial Sequence
  Cell penetrating peptide
SHLIHIWFQNRRLKWKKK

SEQ ID NO: 126 - protein, 22 aa, Artificial Sequence
  Cell penetrating peptide
MVTVLFRRLRIRRACGPPRVRV

SEQ ID NO: 127 - protein, 17 aa, Artificial Sequence
  Cell penetrating peptide
CSIPPEVKFNKPFVYLI

SEQ ID NO: 128 - protein, 15 aa, Artificial Sequence
  Cell penetrating peptide
SDLWEMMMVSLACQY

SEQ ID NO: 129 - protein, 20 aa, Artificial Sequence
  Cell penetrating peptide
HGLASTLTRWAHYNALIRAF

SEQ ID NO: 130 - protein, 16 aa, Artificial Sequence
  Cell penetrating peptide
YTAIAWVKAFIRKLRK

SEQ ID NO: 131 - protein, 17 aa, Artificial Sequence
  Cell penetrating peptide
IAWVKAFIRKLRKGPLG

SEQ ID NO: 132 - protein, 14 aa, Artificial Sequence
  Cell penetrating peptide
GRKKRRQRRRPPQC

SEQ ID NO: 133 - protein, 18 aa, Artificial Sequence
  Cell penetrating peptide
RQIKIWFQNRRMKWKKGC

SEQ ID NO: 134 - protein, 14 aa, Artificial Sequence
  Cell penetrating peptide
RRRRRRRRRRRRGC

SEQ ID NO: 135 - protein, 18 aa, Artificial Sequence
  Cell penetrating peptide
RRRRRRRRRRRRRRRRGC

SEQ ID NO: 136 - protein, 13 aa, Artificial Sequence
  Cell penetrating peptide
EEEAAGRKRKKRT

SEQ ID NO: 137 - protein, 8 aa, Artificial Sequence
  Cell penetrating peptide
EEEAAKKK

SEQ ID NO: 138 - protein, 8 aa, Artificial Sequence
  Cell penetrating peptide
GRKRKKRT

SEQ ID NO: 139 - protein, 13 aa, Artificial Sequence
  Cell penetrating peptide
FFFAAGRKRKKRT

SEQ ID NO: 140 - protein, 13 aa, Artificial Sequence
  Cell penetrating peptide
NNNAAGRKRKKRT

SEQ ID NO: 141 - protein, 13 aa, Artificial Sequence
  Cell penetrating peptide
YYYAAGRKRKKRT

SEQ ID NO: 142 - protein, 24 aa, Artificial Sequence
  Cell penetrating peptide
RRRRRRRRRGPGVTWTPQAWFQWV

SEQ ID NO: 143 - protein, 15 aa, Artificial Sequence
  Cell penetrating peptide
CGGGYGRKKRRQRRR

SEQ ID NO: 144 - protein, 25 aa, Artificial Sequence
  Cell penetrating peptide
YKQCHKKGGHCFPKEKICLPPSSDF

SEQ ID NO: 145 - protein, 17 aa, Artificial Sequence
  Cell penetrating peptide
GKMDCRWRWKCCKKGSG

SEQ ID NO: 146 - protein, 11 aa, Artificial Sequence
  Cell penetrating peptide
YGRKKRPQRRR

SEQ ID NO: 147 - protein, 15 aa, Artificial Sequence
  Cell penetrating peptide
DSLKSYWYLQKFSWR

SEQ ID NO: 148 - protein, 15 aa, Artificial Sequence
  Cell penetrating peptide
KLWMRWWSPTTRRYG

SEQ ID NO: 149 - protein, 15 aa, Artificial Sequence
  Cell penetrating peptide
RLWMRWYSPWTRRWG

SEQ ID NO: 150 - protein, 15 aa, Artificial Sequence
  Cell penetrating peptide
RLIMRIYAPTTRRYG

SEQ ID NO: 151 - protein, 15 aa, Artificial Sequence
  Cell penetrating peptide
RLYMRYYSPTTRRYG

SEQ ID NO: 152 - protein, 15 aa, Artificial Sequence
  Cell penetrating peptide
RLWMRWYSPRTRAYG

SEQ ID NO: 153 - protein, 15 aa, Artificial Sequence
  Cell penetrating peptide
KRPTMRFRYTWNPMK

SEQ ID NO: 154 - protein, 15 aa, Artificial Sequence
  Cell penetrating peptide
WKCRRQCFRVLHHWN

SEQ ID NO: 155 - protein, 15 aa, Artificial Sequence
  Cell penetrating peptide
WKCRRQAFRVLHHWN

SEQ ID NO: 156 - protein, 15 aa, Artificial Sequence
  Cell penetrating peptide
WKARRQCFRVLHHWN

SEQ ID NO: 157 - protein, 9 aa, Artificial Sequence
  Cell penetrating peptide
CRGDKGPDC

SEQ ID NO: 158 - protein, 25 aa, Artificial Sequence
  Cell penetrating peptide
RRRRRRRGGIYLATALAKWALKQGF

SEQ ID NO: 159 - protein, 25 aa, Artificial Sequence
  Cell penetrating peptide
IYLATALAKWALKQGFGGRRRRRRR

SEQ ID NO: 160 - protein, 23 aa, Artificial Sequence
  Cell penetrating peptide
RRRRRRRGGIYLATALAKWALKQ

SEQ ID NO: 161 - protein, 23 aa, Artificial Sequence
  Cell penetrating peptide
IYLATALAKWALKQGGRRRRRRR

SEQ ID NO: 162 - protein, 23 aa, Artificial Sequence
  Cell penetrating peptide
RRRRRRRGGKLAKLAKKLAKLAK

SEQ ID NO: 163 - protein, 23 aa, Artificial Sequence
  Cell penetrating peptide
KLAKLAKKLAKLAKGGRRRRRRR

SEQ ID NO: 164 - protein, 15 aa, Artificial Sequence
  Cell penetrating peptide
RGDADDARRRRRRRR

SEQ ID NO: 165 - protein, 5 aa, Artificial Sequence
  Cell penetrating peptide
CRGDK

SEQ ID NO: 166 - protein, 11 aa, Artificial Sequence
  Cell penetrating peptide
RGDRRRRRRRR

SEQ ID NO: 167 - protein, 8 aa, Artificial Sequence
  Cell penetrating peptide
RRRRRRRR

SEQ ID NO: 168 - protein, 25 aa, Artificial Sequence
  Cell penetrating peptide
GIGKFLHSAKKFGKAFVGEIMNSGG

SEQ ID NO: 169 - protein, 17 aa, Artificial Sequence
  Cell penetrating peptide
KKWKMRRNQFWVKVQRG

SEQ ID NO: 170 - protein, 24 aa, Artificial Sequence
  Cell penetrating peptide
RRIRPRPPRLPRPRPRPLPFPRPG

SEQ ID NO: 171 - protein, 11 aa, Artificial Sequence
  Cell penetrating peptide
YGRKKRRQRRR

SEQ ID NO: 172 - protein, 14 aa, Artificial Sequence
  Cell penetrating peptide
VSRRRRRRGGRRRR

SEQ ID NO: 173 - protein, 13 aa, Artificial Sequence
  Cell penetrating peptide
CAYGRKKRRQRRR

SEQ ID NO: 174 - protein, 13 aa, Artificial Sequence
  Cell penetrating peptide
CAYGRKKRRQRRR

SEQ ID NO: 175 - protein, 12 aa, Artificial Sequence
  Cell penetrating peptide
CYGRKKRRQRRR

SEQ ID NO: 176 - protein, 15 aa, Artificial Sequence
  Cell penetrating peptide
VKRFKKFFRKLKKSV

SEQ ID NO: 177 - protein, 15 aa, Artificial Sequence
  Cell penetrating peptide
VKRFKKFFRKLKKLV

SEQ ID NO: 178 - protein, 15 aa, Artificial Sequence
  Cell penetrating peptide
VKRFKKFFRKLKKKV

SEQ ID NO: 179 - protein, 13 aa, Artificial Sequence
  Cell penetrating peptide
CAYGRKKRRQRRR

SEQ ID NO: 180 - protein, 13 aa, Artificial Sequence
  Cell penetrating peptide
CAYGRKKRRQRRR

SEQ ID NO: 181 - protein, 8 aa, Artificial Sequence
  Cell penetrating peptide
CHAIYPRH

SEQ ID NO: 182 - protein, 27 aa, Artificial Sequence
  Cell penetrating peptide
GWTLNSAGYLLGKINLKALAALAKKIL

SEQ ID NO: 183 - protein, 27 aa, Artificial Sequence
  Cell penetrating peptide
GWTLNSAGYLLGKINLKALAALAKKIL

SEQ ID NO: 184 - protein, 27 aa, Artificial Sequence
  Cell penetrating peptide
GWTLNSAGYLLGKINLKALAALAKKIL

SEQ ID NO: 185 - protein, 21 aa, Artificial Sequence
  Cell penetrating peptide
AGYLLGKINLKALAALAKKIL

SEQ ID NO: 186 - protein, 21 aa, Artificial Sequence
  Cell penetrating peptide
AGYLLGKINLKALAALAKKIL

SEQ ID NO: 187 - protein, 10 aa, Artificial Sequence
  Cell penetrating peptide
NTCTWLKYHS

SEQ ID NO: 188 - protein, 12 aa, Artificial Sequence
  Cell penetrating peptide
CASGQQGLLKLC

SEQ ID NO: 189 - protein, 10 aa, Artificial Sequence
  Cell penetrating peptide
YNNFAYSVFL

SEQ ID NO: 190 - protein, 10 aa, Artificial Sequence
  Cell penetrating peptide
ECYPKKGQDP

SEQ ID NO: 191 - protein, 10 aa, Artificial Sequence
  Cell penetrating peptide
RHVYHVLLSQ

SEQ ID NO: 192 - protein, 10 aa, Artificial Sequence
  Cell penetrating peptide
HATKSQNINF

SEQ ID NO: 193 - protein, 10 aa, Artificial Sequence
  Cell penetrating peptide
YRDRFAFQPH

SEQ ID NO: 194 - protein, 10 aa, Artificial Sequence
  Cell penetrating peptide
IWRYSLASQQ

SEQ ID NO: 195 - protein, 10 aa, Artificial Sequence
  Cell penetrating peptide
YQKQAKIMCS

SEQ ID NO: 196 - protein, 8 aa, Artificial Sequence
  Cell penetrating peptide
VQLRRRWC

SEQ ID NO: 197 - protein, 9 aa, Artificial Sequence
  Cell penetrating peptide
RRRRRRRRR

SEQ ID NO: 198 - protein, 10 aa, Artificial Sequence
  Cell penetrating peptide
NTGTWLKYHS

SEQ ID NO: 199 - protein, 9 aa, Artificial Sequence
  Cell penetrating peptide
TCTWLKYHS

SEQ ID NO: 200 - protein, 9 aa, Artificial Sequence
  Cell penetrating peptide
NTCTWLKYH

SEQ ID NO: 201 - protein, 8 aa, Artificial Sequence
  Cell penetrating peptide
TCTWLKYH

SEQ ID NO: 202 - protein, 7 aa, Artificial Sequence
  Cell penetrating peptide
CTWLKYH

SEQ ID NO: 203 - protein, 6 aa, Artificial Sequence
  Cell penetrating peptide
TWLKYH

SEQ ID NO: 204 - protein, 6 aa, Artificial Sequence
  Cell penetrating peptide
CTWLKY

SEQ ID NO: 205 - protein, 22 aa, Artificial Sequence
  Cell penetrating peptide
LSTAADMQGVVTDGMASGLDKD

SEQ ID NO: 206 - protein, 6 aa, Artificial Sequence
  Cell penetrating peptide
YLKPDD

SEQ ID NO: 207 - protein, 24 aa, Artificial Sequence
  Cell penetrating peptide
WEARLARALARALARHLARALARA

SEQ ID NO: 208 - protein, 11 aa, Artificial Sequence
  Cell penetrating peptide
YGRKKRRQRRR

SEQ ID NO: 209 - protein, 16 aa, Artificial Sequence
  Cell penetrating peptide
RQIKIWFQNRRMKWKK

SEQ ID NO: 210 - protein, 21 aa, Artificial Sequence
  Cell penetrating peptide
KETWWETWWTEWSQPKKKRKV

SEQ ID NO: 211 - protein, 13 aa, Artificial Sequence
  Cell penetrating peptide
RIMRILRILKLAR

SEQ ID NO: 212 - protein, 10 aa, Artificial Sequence
  Cell penetrating peptide
SRRRRRRRRR

SEQ ID NO: 213 - protein, 21 aa, Artificial Sequence
  Cell penetrating peptide
CHHHHHRRRRRRRRRHHHHHC

SEQ ID NO: 214 - protein, 16 aa, Artificial Sequence
  Cell penetrating peptide
FFLIPKGRRRRRRRRR

SEQ ID NO: 215 - protein, 21 aa, Artificial Sequence
  Cell penetrating peptide
  MISC_FEATURE (10)..(11): Xaa = Pyrrolysine (O)
  MISC_FEATURE (18)..(19): Xaa = Pyrrolysine (O)
AGYLLGKLLXXLAAAALXXLL

SEQ ID NO: 216 - protein, 14 aa, Artificial Sequence
  Cell penetrating peptide
KGRKKRRQRRRPPQ

SEQ ID NO: 217 - protein, 14 aa, Artificial Sequence
  Cell penetrating peptide
KGRKKRRQRRRPPQ

SEQ ID NO: 218 - protein, 21 aa, Artificial Sequence
  Cell penetrating peptide
  MISC_FEATURE (17)..(17): Xaa is aminoisobutyric acid (Aib)
AGYLLGHINLHHLAHLXHHIL

SEQ ID NO: 219 - protein, 10 aa, Artificial Sequence
  Cell penetrating peptide
  MISC_FEATURE (2)..(8): D configuration of of arginine
RRRRRRRRGC

SEQ ID NO: 220 - protein, 8 aa, Artificial Sequence
  Cell penetrating peptide
  MISC_FEATURE (1)..(8): D configuration of arginine
RRRRRRRR

SEQ ID NO: 221 - protein, 14 aa, Artificial Sequence
  Cell penetrating peptide
  MISC_FEATURE (1)..(12): D configuration of arginine
RRRRRRRRRRRRGC

SEQ ID NO: 222 - protein, 16 aa, Artificial Sequence
  Cell penetrating peptide
  MISC_FEATURE (1)..(9): D configuration of arginine
  MISC_FEATURE (10)..(10): D configuration of cysteine
  MISC_FEATURE (11)..(11): D configuration of glutamine
  MISC_FEATURE (12)..(12): D configuration of cysteine
  MISC_FEATURE (13)..(14): D configuration of arginine
  MISC_FEATURE (15)..(15): D configuration of lysine
  MISC_FEATURE (16)..(6): D configuration of asparagine
RRRRRRRRRCQCRRKN